(19) United States
(12) Patent Application Publication
(10) Pub. No.: US 2026/0325561 A1
(43) Pub. Date: Oct. 22, 2026

(54) 4'-SUBSTITUTED NUCLEOSIDES AND NUCLEOTIDES AS ANTIVIRAL AGENTS

(71) Applicant: THE SCRIPPS RESEARCH INSTITUTE, La Jolla, CA (US)

(72) Inventors: Chenguang YU, San Diego, CA (US); Arnab CHATTERJEE, San Diego, CA (US); Laura RIVA, San Diego, CA (US); Karen C. WOLFF, Encinitas, CA (US); Case W. McNAMARA, San Marcos, CA (US); Hengyao YOU, San Diego, CA (US); Anil Kumar GUPTA, San Diego, CA (US); Omar SALEH, San Diego, CA (US)

(73) Assignee: THE SCRIPPS RESEARCH INSTITUTE, La Jolla, CA (US)

(21) Appl. No.: 19/478,897

(22) PCT Filed: Apr. 29, 2024
(86) PCT No.: PCT/US2024/026825

§ 371 Date: Oct. 27, 2025

Related U.S. Application Data
(60) Provisional application No. 63/498,910, filed on Apr. 28, 2023.

(51) Int. Cl. A61K 31/7064; A61K 31/403; A61K 31/706; A61K 31/7068; A61P 31/14; C07H 19/04

(52) U.S. Cl. CPC A61K 31/7064; A61K 31/403; A61K 31/706; A61K 31/7068; A61P 31/14; C07H 19/04

(57) ABSTRACT

The present application provides nucleoside analogue compounds of Formulae I-V for the treatment of Dengue Fever (DF). The present application further provides compositions and combinations thereof as well as methods of treatment of Dengue Fever using the nucleoside compounds of Formula I-V and compositions and combinations thereof.

### CROSS REFERENCE TO RELATED APPLICATION

This application claims priority to U.S. provisional patent application No. 63/498,910, which was filed on Apr. 28, 2023, and which is hereby incorporated by reference in its entirety.

### FIELD OF THE INVENTION

The present application provides nucleoside analogue compounds of Formulae I-V for the treatment of Dengue Fever (DF). The present application further provides compositions and combinations thereof as well as methods of treatment of dengue fever using the nucleoside compounds of Formula I-V and compositions and combinations thereof.

### BACKGROUND OF THE INVENTION

Dengue fever is an acute febrile disease caused by one of four closely related virus serotypes (DENV-1, DENV-2, DENV-3, and DENV-4). Dengue fever is classified based on its clinical characteristics into classical dengue fever, or the more severe forms, dengue hemorrhagic fever syndrome (DHF), and dengue shock syndrome (DSS). Recovery from infection from one serotype produces life-long immunity to that particular serotype, but provides only short-lived and limited protection against any of the other serotypes. Dengue is a member of the Flaviviridae family which are enveloped, positive-sense RNA viruses whose human pathogens also include West Nile virus (WNV), yellow fever virus (YFV), Japanese encephalitis virus (JEV), and tick-borne encephalitis virus (TBEV) among others. Dengue transmission mainly occurs via the bite of an infected Aedes aegypti mosquito which is currently found in tropical and sub-tropical regions around the world.

Each year regional dengue epidemics cause significant morbidity and mortality, social disruption and substantial economic burden on the societies affected both in terms of hospitalization and mosquito control. Dengue is considered by the World Health Organization (WHO) to be the most important arthropod-borne viral disease with an estimated 50 million cases of dengue infection, including 500,000 DHF cases and 24,000 deaths worldwide each year. WHO estimates that forty percent of the world's population (2.5 billion people) are at risk for DF, DHF, and DSS. Dengue is also a NIAID Category A pathogen and in terms of bio-defense, represents a significant threat to United States troops overseas. Dengue is an emerging threat to North America with a dramatic increase in severe disease in the past 25 years including major epidemics in Cuba and Venezuela, and outbreaks in Texas and Hawaii.

Failure to control the mosquito vector and increases in long-distance travel have contributed to the increase and spread of dengue disease. The characteristics of dengue as a viral hemorrhagic fever virus (arthropod-borne, widely spread, and capable of inducing a great amount of cellular damage and eliciting an immune response that can result in severe hemorrhage, shock, and death) makes it a unique threat to deployed military personnel around the world as well as to travelers to tropical regions. Preparedness for both biodefense and for the public health challenges posed by dengue will require the development of new vaccines and antiviral therapeutics.

Dengue causes several illnesses with increasing severity being determined in part by prior infection with a different serotype of the virus. Classic dengue fever (DF) begins 3-8 days after the bite of an infected mosquito and is characterized by sudden onset of fever, headache, back pain, joint pain, a measles-like rash, and nausea and vomiting. DF is frequently referred to as “breakbone” fever due to these symptoms. The disease usually resolves after two weeks but a prolonged recovery with weakness and depression is common.

DHF, the more severe form of the disease, has a similar onset and early phase of illness as dengue fever. However, shortly after onset, the disease is characterized by high fever, enlargement of the liver, and hemorrhagic phenomena such as bleeding from the nose, mouth, and internal organs due to vascular permeability. In DSS, circulatory failure and hypovolemic shock resulting from plasma leakage occur and can lead to death in 12-24 hours without plasma replacement. The case fatality rate of DHF/DSS can be as high as 20% without treatment. DHF has become a leading cause of hospitalization and death among children in many countries with an estimated 500,000 cases requiring hospitalization each year and a case fatality rate of about 5%.

The pathogenesis of DHF/DSS is still being studied but is thought to be in part associated to an enhancement of virus replication in macrophages by heterotypic antibodies, termed antibody-dependent enhancement (ADE). Specifically, during a secondary infection with a different serotype of dengue virus, cross-reactive antibodies that are not neutralizing, form virus-antibody complexes that favorize the Fc-mediated viral uptake into monocytes and Langerhans cells (dendritic cells), increasing the number of infected cells. This leads to the activation of cytotoxic lymphocytes which can result in plasma leakage and hemorrhagic features characteristic of DHF and DSS. This antibody-dependent enhancement of infection is one reason why the development of a successful vaccine has proven to be so difficult. Although less frequent, DHF/DSS can occur after primary infection, so virus virulence and immune activation are also believed to contribute to the pathogenesis of the disease.

Dengue is endemic in more than 100 countries in Africa, the Americas, the Eastern Mediterranean, South-east Asia and the Western Pacific. During epidemics, attack rates can be as high as 80-90% of the susceptible population. All four serotypes of the virus are emerging worldwide, increasing the number of cases of the disease as well as the number of explosive outbreaks. In 2002 for example, there were 1,015,420 reported cases of dengue in the Americas alone with 14,374 cases of DHF, which is more than three times the number of dengue cases reported in the Americas in 1995.

The dengue genome consists of a linear, single stranded, positive sense RNA of approximately 11 kb in length. This genome is capped and does not have a poly (A) tail at the 3′ end but instead has a stable stem-loop structure necessary for stability and replication of the viral genomic RNA. The three structural proteins namely the nucleocapsid protein (C), the membrane-associated protein (M), and the envelope protein (E), surround the viral RNA and constitute the virion.

During the infection, the virus binds to cellular receptors via the E protein and undergoes receptor-mediated endocytosis followed by low-pH fusion in lysosomes. The virion is then uncoated and the viral RNA is released in the cytoplasm and translated into a single viral precursor polyprotein. The polyprotein is composed of the three structural proteins C, M and E, and seven nonstructural (NS) proteins. The precursor polyprotein is cleaved by cellular proteinases to separate the structural proteins, while a virus-encoded proteinase cleaves the nonstructural region of the polyprotein. Both co- and posttranslational proteolytic processing separates the viral proteins. Specifically, the structural proteins are involved mainly in viral particle formation, while the nonstructural proteins are involved in viral RNA replication and viral assembly, as well as immune-modulation and contribution to disease pathogenesis.

The non-structural protein 5 (NS5) constitutes the RNA-dependent RNA polymerase which, along with cofactors, synthesizes the minus-strand RNA which serves as a template for the synthesis of the progeny plus-strand RNAs. Viral replication is membrane associated and occurs in specific endoplasmic reticulum (ER)-derived subcellular compartments. Following replication, the genome is encapsidated, and the immature virus, surrounded by a lipid envelope, buds into the ER lumen, traffics through the TGN where the envelope proteins become glycosylated, then mature viruses are finally released at the cell surface.

Essential stages or processes during the virus life cycle could be possible targets for inhibition from an antiviral drug and include binding of the virus to the cell through the E protein, uptake of the virus into the cell, the capping mechanism, the viral proteinase, the viral RNA-dependent RNA polymerase, and the viral helicase.

Current management of dengue virus-related disease relies solely on vector control. There are no approved antivirals or vaccines for the treatment or prevention of dengue.

Ribavirin, a guanosine analogue, has been shown to be effective against a range of RNA virus infections and works against dengue in tissue culture by inhibiting the dengue 2′-0-methyltransferase NS5 domain. However, ribavirin did not show protection against dengue in a mouse model or a rhesus monkey model, instead it induced anemia and thrombocytosis. Although some antiviral candidates targeting DENV NS4B are currently in clinical trials (i.e. JNJ-1802, NITD-688), no treatment has been approved yet, and the chances of appearance of drug-resistance to direct-acting antivirals represent a serious and constant threat for RNA viruses. This can only be overcome by the development of antiviral molecules with alternative mechanisms of action, hence the urgence to further develop antiviral candidates targeting this pathogen.

While there are currently two available approved vaccines (Dengvaxia and QDENGA). Dengvaxia is only recommended to people who previously had dengue fever. QDENGA was recently approved in Indonesia and European Union. Overall, multivalent dengue vaccines have shown some limited potential in humans, due to the difficulties represented by the presence of four distinct serotypes of the virus which each cause disease. Vaccine development also faces the challenge of ADE where unequal protection against the four serotypes of the virus could actually increase the risk of more serious disease.

Therefore, there is a need for antiviral drugs that target all the serotypes of dengue. An antiviral drug administered early during dengue infection that inhibits viral replication would prevent the high viral load associated with DHF and be an attractive strategy in the treatment and prevention of the disease. An antiviral drug that inhibits viral replication could be administered as prophylaxis prior to travel to a dengue endemic region to prevent acquisition of disease, or for those that have previously been exposed to dengue, could prevent infection by another viral serotype and decrease the chance of life-threatening DHF and DSS. Having an antiviral drug would also aid vaccine development by having a tool at hand to treat complications that may arise due to unequal immune protection against the different serotypes. Although a successful vaccine could be a critical component of an effective biodefense, the typical delay to onset of immunity, potential side-effects, cost, and logistics associated with large-scale civilian vaccinations against a low-threat risk agent suggest that a comprehensive biodefense includes a separate rapid-response element.

There thus exists a clear and long-felt need to develop effective therapeutics for treatment of dengue virus. Specifically, there is a need to develop compounds that are useful for treating dengue-infected patients and compounds that selectively inhibit dengue viral replication.

### BRIEF DESCRIPTION OF THE FIGURES

FIG. 1. Summary of antiviral activity of Example 1 and other known Nucs.

FIG. 2. Comparison of Select Antiviral Activity of Compound 58 vs. AL-611.

### SUMMARY OF THE INVENTION

The disclosure provides a compound of Formula (I)

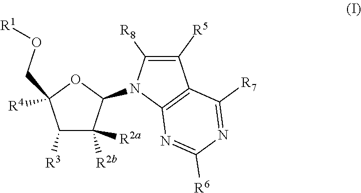

wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R^{2a} is H, halo, (C₁-C₆)alkyl, or —C≡CH;
R^{2b} is independently halo or OH;
R³ is independently H or OH;
R⁴ is N₃, halo, —C≡N, (C₁-C₃)haloalkyl or —O(C₁-C₆)alkyl;
R⁵ is H, halo, —C≡N, (C₁-C₆)alkyl, hetero (C₁-C₆)alkyl, hydroxy (C₁-C₆)alkyl, N(R¹′)₂, —C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, oxo, N(R′)₂ or —O(C₁-C₆)alkyl; and
R⁸ is H or halo;
   with the proviso that the compound of Formula I is not 4-amino-7-((2R,3R,4S,5S)-5-fluoro-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5R)-5-azido-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carbonitrile; ((5-(4-amino-7H-pyrrolo[2,3-d]pyrimidin-7-yl)-4-ethynyl-2-fluoro-3,4-dihydroxytetrahydrofuran-2-yl)methyl)triphosphoric acid; ((5-(4-amino-5-fluoro-7H-pyrrolo[2,3-d]pyrimidin-7-yl)-4-ethynyl-2-fluoro-3,4-dihydroxytetrahydrofuran-2-yl)methyl)triphosphoric acid; (5-(4-amino-5-carbamoyl-7H-pyrrolo[2,3-d]pyrimidin-7-yl)-4-ethynyl-2-fluoro-3,4-dihydroxytetrahydrofuran-2-yl)methyl tetrahydrogen triphosphate; (5-(4-amino-5-cyano-7H-pyrrolo[2,3-d]pyrimidin-7-yl)-4-ethynyl-2-fluoro-3,4-dihydroxytetrahydrofuran-2-yl)methyl tetrahydrogen triphosphate; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide; (2S,3S,4R,5R)-5-(4-amino-7H-pyrrolo[2,3-d]pyrimidin-7-yl)-4-ethynyl-2-fluoro-2-(hydroxymethyl)tetrahydrofuran-3,4-diol; or (2S,3 S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol; including enantiomers, racemic and scalemic mixtures, and further including pharmaceutically acceptable salts thereof.

The disclosure further provides a compound having the formula (2S,3S,4R,5R)-5-(4-amino-7H-pyrrolo[2,3-d]pyrimidin-7-yl)-2-fluoro-2-(hydroxymethyl)tetrahydrofuran-3,4-diol.

The disclosure further provides a compound having the formula isopropyl ((R)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate.

The disclosure further provides a pharmaceutical composition comprising the compound of any one of Embodiments 1-38 (infra), admixed with a pharmaceutically acceptable carrier, diluent, or excipient.

The disclosure further provides the above pharmaceutical composition, further comprising one or more therapeutic compounds or compositions.

The disclosure further provides the above pharmaceutical composition, wherein the one or more therapeutic compounds or compositions is a second antiviral compound or composition.

The disclosure further provides a method of inhibiting RNA-dependent RNA polymerases, comprising administering to a subject in need thereof a therapeutically effective amount of the compound of any one of Embodiments 1-38 (infra) or the pharmaceutical compositions of Embodiments 39-43 (infra).

The disclosure further provides a method of preventing, ameliorating, or treating an RNA viral infection, comprising administering to a subject in need thereof a therapeutically effective amount of the compound of any one of Embodiments 1-38 (infra) or the pharmaceutical compositions of Embodiments 39-43 (infra).

The disclosure further provides the above method, wherein the RNA viral infection is at least one virus selected from the group consisting of dengue virus, South Asian respiratory syndrome-Coronavirus (SARS-CoV), SARS-CoV-2, zika virus, yellow Fever virus, Ebola (Makona) virus, Ebola (Kikwit) virus, Bundibugyo virus, Sudan virus, Marburg virus, respiratory syncytial virus (RSV), Nipah virus, measles virus, parainfluenza virus, Middle Eastern Respiratory Syndrome (MERS) virus, hepatitis C virus (HCV), West Nile virus, Lassa virus, influenza, HRV, MEV, LCMV, polio, CHIKV, COXV and Junin virus.

The disclosure further provides the above method, wherein the RNA viral infection is caused by dengue fever virus, SARS-CoV-2 virus, yellow fever virus, or zika virus.

### DETAILED DESCRIPTION OF THE INVENTION

RNA-dependent RNA polymerase (RdRp) is an important therapeutic target for treating diseases caused by RNA viruses since RdRp is an essential enzyme for replication of the viral RNA genome and the host lacks a functional equivalent. Nucleoside and nucleotide analogs are well reported as successful antiviral strategy (i.e. Sofosbuvir and Remdesivir) targeting RdRp. Nucleosides analogs are transformed to active 5′-triphosphate metabolites, which, in turn, inhibit the viral replication.

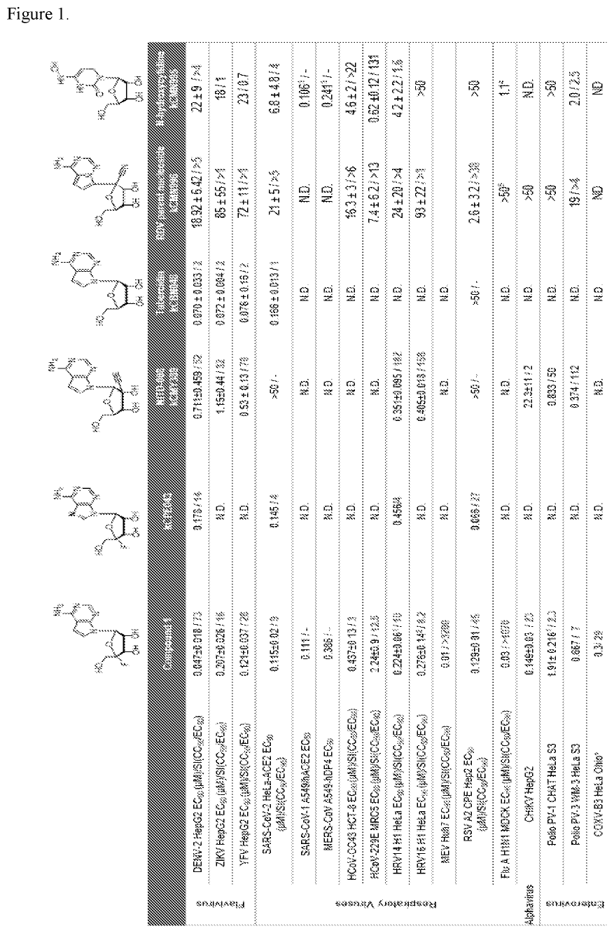

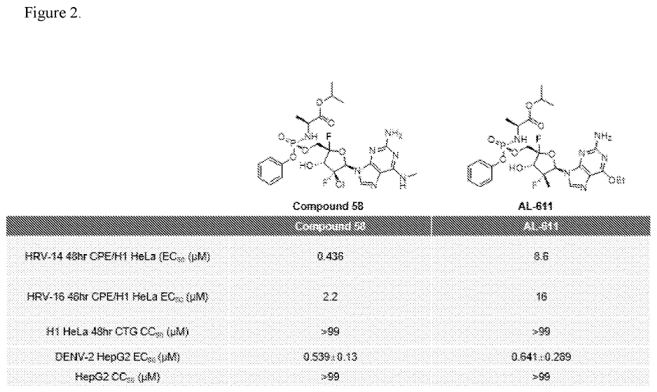

Herein, we disclose our findings of 4′-substituted nucleosides and nucleotides with good antiviral activities against dengue virus (DENV) and severe acute respiratory syndrome coronavirus 2 (SARS-CoV-2). Tubercidin (kCBN048) was identified with high antiviral potency against DENV and SARS-CoV-2 but significant cytotoxicity from screening. NITD-008 (kCMY389) is a reported tubercidin analog with 2′-p-methyl substitution and showed improved selectivity compared to tubercidin (SI_{kCMY389}=52 vs SI_{kCBN048}=2, DENV-2 HepG2 assay, FIG. 1). NITD-008 showed good efficacy in DENV-infected mouse model but failed in two-week tox study.¹ Our strategy is to utilize 4′-substitution to improve the selectivity and activity of tubercidin. Compound 1 with 4′-F showed improved activities and selectivity compared to tubercidin and NITD-008 (SI_{Compound 1}=:73 vs SI_{kCBN048}=2, SI_{kCMY389}=52; DENV2 HepG2 assay, Table 1). Compound 1 and its analogs may be used as a treatment for DENV, COVID-19 and other RNA virus infections. SAR studies around Nucleobase, sugar ring and prodrug strategy are ongoing to improve selectivity, activities, and physicochemical properties. For example, Compound 1 was found to be more efficacious than RDV parent nucleosides and N-hydroxycytidine in Flaviviruses (DENV-2, ZIKV, YFV), Respiratory viruses (SARS-CoV-2, SARS-CoV-1, MERS-CoV, HCoV-OC43, HCoV-229E, HRV14, HRV16, MEV, RSV A2, Flu A H1N1, alphavirus CHIKV) and Enteroviruses (Polio PV-1, Polio PV-3, COXV-B3) (FIG. 1). Additionally, Compound 58 was found to be more efficacious than AL-611 in HRV-14, HRV-16, and DENV-2 (FIG. 2).

There is no antiviral treatment for dengue on the market. Although remdesivir and molnupiravir were approved by FDA as antiviral treatment of COVID-19 with the same MOA, remdesivir must be administered by intravenous (IV) infusion and molnupiravir was reported with potential mutagenic toxicity. New antivirals with better safety and patient compliance (oral drugs) are needed. Lead Compound 1 showed improved activities and selectivity compared to known compounds with the same mechanism of action (MOA) for DENV and SARS-CoV-2.

### EMBODIMENTS

Embodiment 1. A compound of Formula (I)

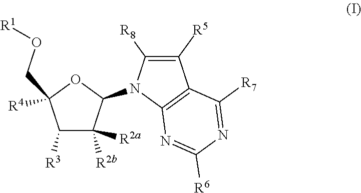

wherein.
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR^{1′})—P(═O)(OR^{1′})₂, —P(═O)(OR¹′)—P(═O)(OR^{1′})—P(═O)(OR^{1′})₂, —P(═O)(OR^{1′})NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R^{2a} is H, halo, (C₁-C₆)alkyl, or —C≡CH;
R^{2b} is independently halo or OH;
R³ is independently H or OH;
R⁴ is N₃, halo, —C≡N, (C₁-C₃)haloalkyl or —O(C₁-C₆)alkyl;
R⁵ is H, halo, —C≡N, (C₁-C₆)alkyl, hetero (C₁-C₆)alkyl, hydroxy (C₁-C₆)alkyl, N(R¹′)₂, —C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, oxo, N(R¹′)₂ or —O(C₁-C₆)alkyl; and
R⁸ is H or halo;
with the proviso that the compound of Formula I is not 4-amino-7-((2R,3R,4S,5S)-5-fluoro-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5R)-5-azido-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carbonitrile; ((5-(4-amino-7H-pyrrolo[2,3-d]pyrimidin-7-yl)-4-ethynyl-2-fluoro-3,4-dihydroxytetrahydrofuran-2-yl)methyl)triphosphoric acid; ((5-(4-amino-5-fluoro-7H-pyrrolo[2,3-d]pyrimidin-7-yl)-4-ethynyl-2-fluoro-3,4-dihydroxytetrahydrofuran-2-yl)methyl)triphosphoric acid; (5-(4-amino-5-carbamoyl-7H-pyrrolo[2,3-d]pyrimidin-7-yl)-4-ethynyl-2-fluoro-3,4-dihydroxytetrahydrofuran-2-yl)methyl tetrahydrogen triphosphate; (5-(4-amino-5-cyano-7H-pyrrolo[2,3-d]pyrimidin-7-yl)-4-ethynyl-2-fluoro-3,4-dihydroxytetrahydrofuran-2-yl)methyl tetrahydrogen triphosphate; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide; (2S,3S,4R,5R)-5-(4-amino-7H-pyrrolo[2,3-d]pyrimidin-7-yl)-4-ethynyl-2-fluoro-2-(hydroxymethyl)tetrahydrofuran-3,4-diol; or (2S,3 S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol; including enantiomers, racemic and scalemic mixtures, and further including pharmaceutically acceptable salts thereof.

Embodiment 2. A compound of Formula (II)

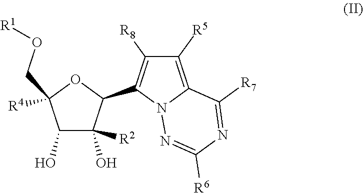

wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR¹′)NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R² is H, (C₁-C₆)alkyl, or —C≡CH;
R⁴ is N₃, halo, or —O(C₁-C₆)alkyl;
R⁵ is H, halo, —C≡N, hetero (C₁-C₆)alkyl, hydroxy (C₁-C₆)alkyl, N(R¹′)₂, —C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, N(R¹′)₂ or —O(C₁-C₆)alkyl; and
R⁸ is H or halo;
with the proviso that the compound of Formula II is not (2S,3S,4R,5S)-5-(4-aminopyrrolo[2,1-f][1,2,4]triazin-7-yl)-2-fluoro-2-(hydroxymethyl)tetrahydrofuran-3,4-diol, (2R,3 S,4R,5S)-5-(4-aminopyrrolo[2,1-f][1,2,4]triazin-7-yl)-2-azido-2-(hydroxymethyl)tetrahydrofuran-3,4-diol, (2R,3S,4R,5S)-5-(4-amino-5-fluoropyrrolo[2,1-f][1,2,4]triazin-7-yl)-2-azido-2-(hydroxymethyl)tetrahydrofuran-3,4-diol; (2R,3S,4R,5S)-5-(4-aminopyrrolo[2,1-f][1,2,4]triazin-7-yl-5-d)-2-azido-2-(hydroxymethyl)tetrahydrofuran-3,4-diol; ((2R,3S,4R,5S)-5-(4-aminopyrrolo[2,1-f][1,2,4]triazin-7-yl)-2-azido-3,4-dihydroxytetrahydrofuran-2-yl)methyl tetrahydrogen triphosphate, or 2-ethylbutyl((((2R,3S,4R,5S)-5-(4-aminopyrrolo[2,1-f][1,2,4]triazin-7-yl)-2-azido-3,4-dihydroxytetrahydrofuran-2-yl)methoxy)(phenoxy)phosphoryl)-D-alaninate; including enantiomers, racemic and scalemic mixtures, and further including pharmaceutically acceptable salts thereof.

Embodiment 3. A compound of Formula (IIIa) or (IIIb)

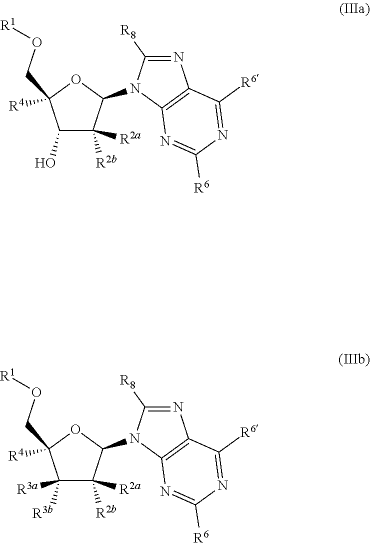

wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅—C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R^{2a} is H, OH, halo, (C₁-C₆)alkyl, or —C≡CH;
R^{2b} is H, OH, halo, or (C₁-C₆)alkyl;
R^{3a} is H, OH, halo, or (C₁-C₆)alkyl;
R^{3b} is H, OH, halo, or (C₁-C₆)alkyl;
R⁴ is N₃, halo, —C≡N, (C₁-C₃)haloalkyl or —O(C₁-C₆)alkyl;
R⁶ and R^{6′} are each independently halo, NH₂, NH(C₁-C₆)alkyl, N((C₁-C₆)alkyl)₂, —OH, —O(C₁-C₆)alkyl, -oxo, or —C≡CH; and
R⁸ is H or halo;
with the proviso that the compound of Formula III is not (((2R,3S,4R,5R)-5-(2,6-diamino-9H-purin-9-yl)-2-(difluoromethyl)-3,4-dihydroxytetrahydrofuran-2-yl)methyl)triphosphoric acid; (((2S,3S,4R,5R)-5-(6-amino-2-fluoro-9H-purin-9-yl)-4-ethynyl-2-fluoro-3,4-dihydroxytetrahydrofuran-2-yl)methyl)triphosphoric acid; (2S,3S,4R,5R)-5-(2,6-diamino-9H-purin-9-yl)-2-fluoro-2-(hydroxymethyl)-4-methyltetrahydrofuran-3,4-diol; or (2S,3S,4R,5R)-5-(6-amino-2-fluoro-9H-purin-9-yl)-4-ethynyl-2-fluoro-2-(hydroxymethyl)tetrahydrofuran-3,4-diol;
including enantiomers, racemic and scalemic mixtures, and further including pharmaceutically acceptable salts thereof.

Embodiment 4. A compound of Formula (IV)

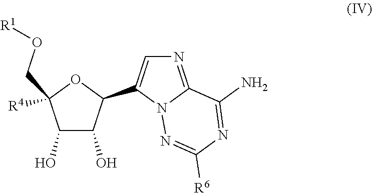

wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR^{1′})NH(C₁-C₆)alkyl, —P(═O)(OR¹′)NH(C₁-C₆)heteroalkyl, —P(═O)(OR^{1′})₂, —P(═O)(OR^{1′})—P(═O)(OR^{1′})₂, —P(═O)(OR¹′)—P(═O)(OR¹′)—P(═O)(OR¹′)₂, P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR^{1′})NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR^{1′})NH(C₁-C₆)alkyl, —P(═O)(NHR¹′)NH(C₁-C₆)heteroalkyl, —P(═O)(NHR^{1′})NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R⁴ is N₃, halo, or —O(C₁-C₆)alkyl; and
R⁶ is H, halo, (C₁-C₆)alkyl, (C₂-C₆)alkenyl, or (C₂-C₆)alkynyl;
including enantiomers, racemic and scalemic mixtures, and further including pharmaceutically acceptable salts thereof.

Embodiment 5. A compound of Formulae (V)

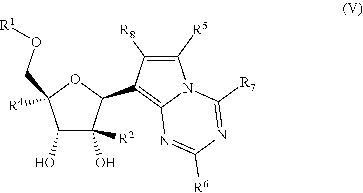

wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R² is H, halo, (C₁-C₆)alkyl, or —C≡CH;
R⁴ is N₃, halo, —C≡N, (C₁-C₃)haloalkyl or —O(C₁-C₆)alkyl;
R⁵ is H, halo, —C≡N, hetero (C₁-C₆)alkyl, hydroxy (C₁-C₆)alkyl, halo (C₁-C₆)alkyl, N(R¹′)₂, —(C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, N(R¹′)₂, or —O(C₁-C₆)alkyl; and
R⁸ is H or halo;
including enantiomers, racemic and scalemic mixtures, and further including pharmaceutically acceptable salts thereof.

Embodiment 6. The compound of Embodiment 1, wherein R^{2b} is OH or halo.

Embodiment 7. The compound of Embodiment 3, wherein R^{2b} is halo.

Embodiment 8. The compound of Embodiment 7, wherein R^{2a} is Me or halo.

Embodiment 9. The compound of any one of Embodiments 6-8, wherein R^{2a} is H.

Embodiment 10. The compound of any one of Embodiments 6-8, wherein R^{2a} is (C₁-C₆)alkyl.

Embodiment 11. The compound of Embodiment 10, wherein R^{2a} is Me.

Embodiment 12. The compound of any one of Embodiments 6-8, wherein R^{2a} is halo.

Embodiment 13. The compound of Embodiment 12, wherein R^{2a} is Cl.

Embodiment 14. The compound of any one of Embodiments 2 or 5 wherein R² is H.

Embodiment 15. The compound of any one of Embodiments 2 or 5, wherein R² is halo.

Embodiment 16. The compound of Embodiment 15, wherein R² is F.

Embodiment 17. The compound of any one of Embodiments 2 or 5, wherein R² is (C₁-C₆)alkyl.

Embodiment 18. The compound of Embodiment 17, wherein R² is Me.

Embodiment 19. The compound of any one of Embodiments 1-2 or 5, wherein R⁵ is H.

Embodiment 20. The compound of any one of Embodiments 1-2 or 5, wherein R⁵ is —CH₂OH.

Embodiment 21. The compound of any one of Embodiments 1-2 or 5, wherein R⁵ is —C(═O)NH₂.

Embodiment 22. The compound of any one of Embodiments 1, 6-13, or 19-21, wherein R³ is OH.

Embodiment 23. The compound of any one of Embodiments 1, 6-13, or 19-21, wherein R³ is H.

Embodiment 24. The compound of any one of Embodiments 1-23, wherein R⁴ is halo.

Embodiment 25. The compound of Embodiment 24, wherein R⁴ is F.

Embodiment 26. The compound of Embodiment 24, wherein R⁴ is C₁.

Embodiment 27. The compound of any one of Embodiments 1-23 wherein R⁴ is N₃.

Embodiment 28. The compound of any one of Embodiments 1-23, wherein R⁴ is —O(C₁-C₆)alkyl.

Embodiment 29. The compound of Embodiment 28, wherein R⁴ is —OMe.

Embodiment 30. The compound of any one of Embodiments 1-29, wherein R⁶ is H or NH₂ and R^{6′} is —NH(C₁-C₆)alkyl or —O(C₁-C₆)alkyl

Embodiment 31. The compound of any one of Embodiments 1-29, wherein R⁶ is halo.

Embodiment 32. The compound of Embodiment 31, wherein R⁶ is F.

Embodiment 33. The compound of any one of Embodiments 1-29, wherein R⁶ is —C—C≡CH.

Embodiment 34. The compound of any one of Embodiments 1-33, wherein R¹ is H.

Embodiment 35. The compound of any one of Embodiments 1-33, wherein R¹ is —P(═O)(OR¹′)₂, —P(═O)(OR¹′)—P(═O)(OR¹′)₂, or —P(═O)(OR¹′)—P(═O)(OR¹′)—P(═O)(OR¹′)₂.

Embodiment 36. The compound of any one of Embodiments 1-33, wherein R¹ is —P(═O)(OR¹′)NH(C₁-C₆)alkyl-C(═O)O—(C₁-C₆)alkyl.

Embodiment 37. A compound having any one of the formulae selected from the group consisting of:
(2S,3S,4R,5R)-5-(4-amino-7H-pyrrolo[2,3-d]pyrimidin-7-yl)-2-fluoro-2-(hydroxymethyl)tetrahydrofuran-3,4-diol;
(2R,3S,4R,5R)-5-(4-amino-7H-pyrrolo[2,3-d]pyrimidin-7-yl)-2-azido-2-(hydroxymethyl)tetrahydrofuran-3,4-diol;
(2R,3S,4R,5S)-5-(4-aminopyrrolo[2,1-f][1,2,4]triazin-7-yl)-2-(hydroxymethyl)-2-methoxy-4-methyltetrahydrofuran-3,4-diol;
(2S,3S,4R,5R)-5-(4-chloro-7H-pyrrolo[2,3-d]pyrimidin-7-yl)-2-fluoro-2-(hydroxymethyl)tetrahydrofuran-3,4-diol;
(2R,3S,4R,5R)-5-(4-chloro-7H-pyrrolo[2,3-d]pyrimidin-7-yl)-2-fluoro-2-(hydroxymethyl)tetrahydrofuran-3,4-diol;
(2S,3R,4S,5R)-5-(4-amino-7H-pyrrolo[2,3-d]pyrimidin-7-yl)-4-chloro-2,4-difluoro-2-(hydroxymethyl)tetrahydrofuran-3-ol;
(2R,3R,4S,5R)-5-(4-amino-7H-pyrrolo[2,3-d]pyrimidin-7-yl)-2-azido-4-chloro-4-fluoro-2-(hydroxymethyl)tetrahydrofuran-3-ol;
(2R,3R,5R)-2-(4-amino-7H-pyrrolo[2,3-d]pyrimidin-7-yl)-5-azido-5-(hydroxymethyl)tetrahydrofuran-3-ol;
(2R,3 S,4R,5S)-5-(4-aminopyrrolo[2,1-f][1,2,4]triazin-7-yl)-2-azido-2-(hydroxymethyl)-4-methyltetrahydrofuran-3,4-diol;
(2S,3S,4R,5R)-5-(4-amino-7H-pyrrolo[2,3-d]pyrimidin-7-yl)-2-fluoro-2-(hydroxymethyl)-4-methyltetrahydrofuran-3,4-diol;
(2R,3R,5R)-2-(4-amino-7H-pyrrolo[2,3-d]pyrimidin-7-yl)-5-fluoro-5-(hydroxymethyl)tetrahydrofuran-3-ol;
(2R,3R,5S)-2-(4-amino-7H-pyrrolo[2,3-d]pyrimidin-7-yl)-5-fluoro-5-(hydroxymethyl)tetrahydrofuran-3-ol;
(2S,3S,4R,5R)-2-fluoro-5-(4-fluoro-7H-pyrrolo[2,3-d]pyrimidin-7-yl)-2-(hydroxymethyl)tetrahydrofuran-3,4-diol;
4-amino-7-((2R,3R,4S,5S)-5-fluoro-3,4-dihydroxy-5-(hydroxymethyl)tetrahydrofuran-2-yl)-7H-pyrrolo[2,3-d]pyrimidine-5-carboxamide;
(2S,3S,4R,5R)-5-(4-amino-5-fluoro-7H-pyrrolo[2,3-d]pyrimidin-7-yl)-2-fluoro-2-(hydroxymethyl)tetrahydrofuran-3,4-diol;
7-((2R,3R,4S,5S)-5-fluoro-3,4-dihydroxy-5-(hydroxymethyl)tetrahydrofuran-2-yl)-3,7-dihydro-4H-pyrrolo[2,3-d]pyrimidin-4-one;
(2S,3S,4R,5R)-5-(4-amino-5-(hydroxymethyl)-7H-pyrrolo[2,3-d]pyrimidin-7-yl)-2-fluoro-2-(hydroxymethyl)tetrahydrofuran-3,4-diol;
(2S,3S,4R,5R)-5-(6-amino-2-ethynyl-9H-purin-9-yl)-2-fluoro-2-(hydroxymethyl)tetrahydrofuran-3,4-diol;
(2S,3S,4R,5S)-5-(4-aminoimidazo[2,1-f][1,2,4]triazin-7-yl)-2-fluoro-2-(hydroxymethyl)tetrahydrofuran-3,4-diol;
(2R,3 S,4R,5R)-5-(4-amino-7H-pyrrolo[2,3-d]pyrimidin-7-yl)-2-(fluoromethyl)-2-(hydroxymethyl)tetrahydrofuran-3,4-diol;
5-fluoro-7-((2R,3R,4S,5S)-5-fluoro-3,4-dihydroxy-5-(hydroxymethyl)tetrahydrofuran-2-yl)-3,7-dihydro-4H-pyrrolo[2,3-d]pyrimidin-4-one;
(2S,3S,4R,5R)-2-fluoro-5-(5-fluoro-4-methoxy-7H-pyrrolo[2,3-d]pyrimidin-7-yl)-2-(hydroxymethyl)tetrahydrofuran-3,4-diol;
(2S,3S,4R,5R)-2-fluoro-2-(hydroxymethyl)-5-(7H-pyrrolo[2,3-d]pyrimidin-7-yl)tetrahydrofuran-3,4-diol;
7-((2R,3R,4S,5S)-5-fluoro-3,4-dihydroxy-5-(hydroxymethyl)tetrahydrofuran-2-yl)-7H-pyrrolo[2,3-d]pyrimidine-5-carboxamide;
(2S,3S,4R,5R)-5-(5-bromo-7H-pyrrolo[2,3-d]pyrimidin-7-yl)-2-fluoro-2-(hydroxymethyl)tetrahydrofuran-3,4-diol;
4-amino-7-((2R,3R,4S,5S)-5-fluoro-3,4-dihydroxy-5-(hydroxymethyl)tetrahydrofuran-2-yl)-7H-pyrrolo[2,3-d]pyrimidine-5-carbonitrile;
(2S,3S,4R,5R)-5-(2-amino-4-chloro-7H-pyrrolo[2,3-d]pyrimidin-7-yl)-2-fluoro-2-(hydroxymethyl)tetrahydrofuran-3,4-diol;
(2S,3S,4R,5R)-2-fluoro-2-(hydroxymethyl)-5-(6-(methylthio)-9H-purin-9-yl)tetrahydrofuran-3,4-diol;
(2S,3S,4R,5R)-5-(4-chloro-2-methyl-7H-pyrrolo[2,3-d]pyrimidin-7-yl)-2-fluoro-2-(hydroxymethyl)tetrahydrofuran-3,4-diol;
(1S,2S,3S,5R)-5-(4-amino-7H-pyrrolo[2,3-d]pyrimidin-7-yl)-3-fluoro-3-(hydroxymethyl)cyclopentane-1,2-diol;
(2S,3S,4R,5R)-5-(6-amino-2-fluoro-9H-purin-9-yl)-2-fluoro-2-(hydroxymethyl)tetrahydrofuran-3,4-diol;
(3S,4R,5R)-5-(4-amino-7H-pyrrolo[2,3-d]pyrimidin-7-yl)-3,4-dihydroxy-2-(hydroxymethyl)tetrahydrofuran-2-carbonitrile;
(2S,3S,4R,5R)-5-(4-amino-5-chloro-7H-pyrrolo[2,3-d]pyrimidin-7-yl)-2-fluoro-2-(hydroxymethyl)tetrahydrofuran-3,4-diol;
((2S,3S,4R,5S)-5-(4-amino-5-carbamoylpyrrolo[2,1-f][1,2,4]triazin-7-yl)-2-fluoro-3,4-dihydroxytetrahydrofuran-2-yl)methyl benzoate;
(2S,3S,4R,5R)-2-fluoro-5-(5-fluoro-4-(methylamino)-7H-pyrrolo[2,3-d]pyrimidin-7-yl)-2-(hydroxymethyl)tetrahydrofuran-3,4-diol;
(2S,3S,4R,5R)-5-(5-chloro-7H-pyrrolo[2,3-d]pyrimidin-7-yl)-2-fluoro-2-(hydroxymethyl)tetrahydrofuran-3,4-diol;
7-((2R,3R,4S,5S)-5-fluoro-3,4-dihydroxy-5-(hydroxymethyl)tetrahydrofuran-2-yl)-4-oxo-4,7-dihydro-3H-pyrrolo[2,3-d]pyrimidine-5-carbonitrile;
7-((2R,3R,4S,5S)-5-fluoro-3,4-dihydroxy-5-(hydroxymethyl)tetrahydrofuran-2-yl)-4-methoxy-7H-pyrrolo[2,3-d]pyrimidine-5-carboxamide;
(2R,3R,5R)-5-fluoro-5-(hydroxymethyl)-2-(4-methoxy-7H-pyrrolo[2,3-d]pyrimidin-7-yl)tetrahydrofuran-3-ol;
(2S,3S,4R,5R)-2-fluoro-2-(hydroxymethyl)-5-(4-methoxy-7H-pyrrolo[2,3-d]pyrimidin-7-yl)tetrahydrofuran-3,4-diol;
(2S,3S,4R,5R)-5-(4-amino-5-(trifluoromethyl)-7H-pyrrolo[2,3-d]pyrimidin-7-yl)-2-fluoro-2-(hydroxymethyl)tetrahydrofuran-3,4-diol;
(2S,3S,4R,5R)-5-(6-amino-2-chloro-9H-purin-9-yl)-2-fluoro-2-(hydroxymethyl)tetrahydrofuran-3,4-diol;
4-amino-1-((2R,3R,4S,5S)-3-ethynyl-5-fluoro-3,4-dihydroxy-5-(hydroxymethyl)tetrahydrofuran-2-yl)pyrimidin-2(1H)-one;
(2S,3S,4R,5R)-2-((benzoyloxy)methyl)-5-(3,5-dioxo-4,5-dihydro-1,2,4-triazin-2(3H)-yl)-2-fluorotetrahydrofuran-3,4-diyl diacetate;
((2S,3S,4R,5R)-5-(4-amino-7H-pyrrolo[2,3-d]pyrimidin-7-yl)-2-fluoro-3,4-dihydroxytetrahydrofuran-2-yl)methyl isobutyrate;
(2S,3S,4R,5R)-5-(4-amino-7H-pyrrolo[2,3-d]pyrimidin-7-yl)-2-fluoro-2-((isobutyryloxy)methyl)tetrahydrofuran-3,4-diyl bis(2-methylpropanoate);
isopropyl ((S)-(((2S,3S,4R,5R)-5-(4-amino-7H-pyrrolo[2,3-d]pyrimidin-7-yl)-2-fluoro-3,4-dihydroxytetrahydrofuran-2-yl)methoxy)(phenoxy)phosphoryl)-L-alaninate;
isopropyl ((R)-(((2S,3S,4R,5R)-5-(4-amino-7H-pyrrolo[2,3-d]pyrimidin-7-yl)-2-fluoro-3,4-dihydroxytetrahydrofuran-2-yl)methoxy)(phenoxy)phosphoryl)-L-alaninate;
((2S,3S,4R,5R)-4-(benzoyloxy)-5-(4-cyano-7H-pyrrolo[2,3-d]pyrimidin-7-yl)-2-fluoro-3-hydroxytetrahydrofuran-2-yl)methyl benzoate;
(2R,3R,4S,5S)-2-(4-aminoimidazo[2,1-f][1,2,4]triazin-7-yl)-5-fluoro-3,4-dihydroxy-5-(hydroxymethyl)tetrahydrofuran-2-carbonitrile;
(2S,3S,4R,5R)-5-(4-amino-1H-pyrazolo[3,4-d]pyrimidin-1-yl)-2-fluoro-2-(hydroxymethyl)tetrahydrofuran-3,4-diol;
(2S,3S,4R,5R)-2-fluoro-2-(hydroxymethyl)-5-(4-methyl-7H-pyrrolo[2,3-d]pyrimidin-7-yl)tetrahydrofuran-3,4-diol;
(2S,3S,4R,5R)-5-(4-cyclopropyl-7H-pyrrolo[2,3-d]pyrimidin-7-yl)-2-fluoro-2-(hydroxymethyl)tetrahydrofuran-3,4-diol;
(2S,3R,5R)-5-(4-amino-5-(benzo[d]thiazol-2-yl)-7H-pyrrolo[2,3-d]pyrimidin-7-yl)-2,4,4-trifluoro-2-(hydroxymethyl)tetrahydrofuran-3-ol;
((2S,3S,4R,5R)-5-(2-amino-4-(methylamino)-7H-pyrrolo[2,3-d]pyrimidin-7-yl)-2-fluoro-3,4-dihydroxytetrahydrofuran-2-yl)methyl benzoate;
(2S,3R,4S,5R)-5-(2-amino-6-(methylamino)-9H-purin-9-yl)-4-chloro-2,4-difluoro-2-(hydroxymethyl)tetrahydrofuran-3-ol;
isopropyl ((R)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate;
isopropyl ((S)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate;
(2S,3R,5R)-5-(2-amino-6-ethoxy-9H-purin-9-yl)-2,4,4-trifluoro-2-(hydroxymethyl)tetrahydrofuran-3-ol;
isopropyl ((((2S,3R,5R)-5-(2-amino-6-oxo-1,6-dihydro-9H-purin-9-yl)-2,4,4-trifluoro-3-hydroxytetrahydrofuran-2-yl)methoxy)(phenoxy)phosphoryl)-L-alaninate;
2-amino-9-((2R,3R,4R,5 S)-3-chloro-5-fluoro-4-hydroxy-5-(hydroxymethyl)-3-methyltetrahydrofuran-2-yl)-1,9-dihydro-6H-purin-6-one;
(2S,3R,4R,5R)-5-(2-amino-6-ethoxy-9H-purin-9-yl)-4-chloro-2-fluoro-2-(hydroxymethyl)-4-methyltetrahydrofuran-3-ol;
isopropyl ((((2S,3R,4R,5R)-5-(2-amino-6-ethoxy-9H-purin-9-yl)-4-chloro-2-fluoro-3-hydroxy-4-methyltetrahydrofuran-2-yl)methoxy)(phenoxy)phosphoryl)-L-alaninate;
neopentyl ((((2S,3S,4R,5R)-5-(4-aminoimidazo[2,1-f][1,2,4]triazin-7-yl)-5-cyano-2-fluoro-3,4-dihydroxytetrahydrofuran-2-yl)methoxy)(naphthalen-1-yloxy)phosphoryl)-L-alaninate;
(2S,3R,4S,5R)-5-(2-amino-6-ethoxy-9H-purin-9-yl)-4-chloro-2,4-difluoro-2-(hydroxymethyl)tetrahydrofuran-3-ol;
isopropyl ((((2S,3R,4S,5R)-5-(2-amino-6-ethoxy-9H-purin-9-yl)-4-chloro-2,4-difluoro-3-hydroxytetrahydrofuran-2-yl)methoxy)(phenoxy)phosphoryl)-L-alaninate;
(2S,3R,4S,5R)-5-(2-amino-6-ethoxy-9H-purin-9-yl)-4-bromo-2,4-difluoro-2-(hydroxymethyl)tetrahydrofuran-3-ol;
isopropyl ((((2S,3R,5R)-5-(2-amino-6-ethoxy-9H-purin-9-yl)-2,4,4-trifluoro-3-hydroxytetrahydrofuran-2-yl)methoxy)(phenoxy)phosphoryl)-L-alaninate;
(2S,3S,4R,5R)-4-ethynyl-2-fluoro-2-(hydroxymethyl)-5-(4-(propylamino)-7H-pyrrolo[2,3-d]pyrimidin-7-yl)tetrahydrofuran-3,4-diol;
2-amino-9-((2R,4R,5S)-3,3,5-trifluoro-4-hydroxy-5-(hydroxymethyl)tetrahydrofuran-2-yl)-1,9-dihydro-6H-purin-6-one;
(2S,3S,4R,5R)-2-fluoro-2-(hydroxymethyl)-5-(4-(methylthio)-7H-pyrrolo[2,3-d]pyrimidin-7-yl)tetrahydrofuran-3,4-diol;
((2S,3S,4R,5R)-5-(4-amino-7H-pyrrolo[2,3-d]pyrimidin-7-yl)-2-fluoro-3,4-dihydroxytetrahydrofuran-2-yl)methyl L-valinate;
neopentyl ((((2S,3S,4R,5R)-5-(4-amino-1H-pyrazolo[3,4-d]pyrimidin-1-yl)-2-fluoro-3,4-dihydroxytetrahydrofuran-2-yl)methoxy)(naphthalen-1-yloxy)phosphoryl)-L-alaninate;
((2S,3S,4R,5R)-5-(4-amino-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(4-chloro-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-2-fluoro-3,4-dihydroxy-5-(4-oxo-3,4-dihydro-7H-pyrrolo[2,3-d]pyrimidin-7-yl)tetrahydrofuran-2-yl)methyl tetrahydrogen triphosphate;
((2S,3S,4R,5R)-5-(5-carbamoyl-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(5-bromo-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-2-fluoro-5-(5-fluoro-4-oxo-3,4-dihydro-7H-pyrrolo[2,3-d]pyrimidin-7-yl)-3,4-dihydroxytetrahydrofuran-2-yl)methyl tetrahydrogen triphosphate;
((2S,3S,4R,5R)-5-(4-aminoimidazo[2,1-f][1,2,4]triazin-7-yl)-5-cyano-2-fluoro-3,4-dihydroxytetrahydrofuran-2-yl)methyl tetrahydrogen triphosphate;
((2S,3S,4R,5R)-5-(4-amino-5-chloro-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(4-amino-5-fluoro-7H-pyrrolo[2,3-d]pyrimidin-7-yl)-2-fluoro-3,4-dihydroxytetrahydrofuran-2-yl)methyl tetrahydrogen triphosphate;
((2S,3R,4R,5R)-5-(2-amino-6-oxo-1,6-dihydro-9H-purin-9-yl)-4-chloro-2-fluoro-3-hydroxy-4-methyltetrahydrofuran-2-yl)methyl tetrahydrogen triphosphate; and
((2S,3S,4S,5R)-5-(4-amino-7H-pyrrolo[2,3-d]pyrimidin-7-yl)-2-fluoro-3,4-dihydroxy-4-methyltetrahydrofuran-2-yl)methyl tetrahydrogen triphosphate.
Embodiment 38. The compound of Embodiment 37 having the formula
(2S,3S,4R,5R)-5-(4-amino-7H-pyrrolo[2,3-d]pyrimidin-7-yl)-2-fluoro-2-(hydroxymethyl)tetrahydrofuran-3,4-diol or isopropyl ((R)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate.

Embodiment 39. A pharmaceutical composition comprising the compound of any one of Embodiments 1-38, admixed with a pharmaceutically acceptable carrier, diluent, or excipient.

Embodiment 40. The pharmaceutical composition of Embodiment 39, further comprising one or more therapeutic compounds or compositions.

Embodiment 41. The pharmaceutical composition of Embodiment 40, wherein the one or more therapeutic compounds or compositions is a second antiviral compound or composition.

Embodiment 42. The pharmaceutical composition of Embodiment 41, wherein the second antiviral compound or composition is an RdRp inhibitor.

Embodiment 43. The pharmaceutical composition of Embodiment 41, wherein the second antiviral compound or composition is and RNA polymerase inhibitor.

Embodiment 44. A method of inhibiting RNA-dependent RNA polymerases, comprising administering to a subject in need thereof a therapeutically effective amount of the compound of any one of Embodiments 1-38 or the pharmaceutical compositions of Embodiments 39-43.

Embodiment 45. A method of preventing, ameliorating, or treating an RNA viral infection, comprising administering to a subject in need thereof a therapeutically effective amount of the compound of any one of Embodiments 1-38 or the pharmaceutical compositions of Embodiments 39-43.

Embodiment 46. The method of embodiment 45, wherein the RNA viral infection is at least one virus selected from the group consisting of dengue virus, South Asian Respiratory syndrome-Coronavirus (SARS-CoV), SARS-CoV-2, zika virus, yellow fever virus, Ebola (Makona) virus, Ebola (Kikwit) virus, Bundibugyo virus, Sudan virus, Marburg virus, respiratory syncytial virus (RSV), Nipah virus, measles virus, parainfluenza virus, Middle Eastern Respiratory Syndrome (MERS) virus, hepatitis C virus (HCV), West Nile virus, Lassa virus, influenza, HRV, MEV, LCMV, polio, CHIKV, COXV and Junin virus.

Embodiment 47. The method of Embodiment 46, wherein the RNA viral infection is caused by dengue fever virus.

Embodiment 48. The method of Embodiment 46, wherein the RNA viral infection is caused by SARS-CoV-2 virus.

Embodiment 49. The method of Embodiment 46, wherein the RNA viral infection is caused by yellow fever virus.

Embodiment 50. The method of Embodiment 46, wherein the RNA viral infection is caused by zika virus.

Embodiment 51. The method of any one of Embodiments 44-50, further comprising treatment with one or more additional therapeutic compounds or compositions.

Embodiment 52. The method of Embodiment 51, wherein at least one of the one or more therapeutic compounds or compositions is a drug effective for treating or ameliorating RNA viral infections.

Embodiment 53. The method of Embodiment 52, wherein the drug for treating an RNA viral infection is selected from the group consisting of remdesivir, molnupiravir, or paxlovid.

Embodiment 54. Any compound, composition, or method as described herein.

### Definitions

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “~~~~” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertible species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄-C(—OH)═CH—), amide/imidic acid (—C(═O)—NH—⇄-C(—OH)═N—) and amidine (—C(═NR)—NH—⇄-C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino,dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR—, —C(═O)R^{aa}, —CO₂H, —CHO, —CO₂Ra, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NRC^{bb}(═O)N(R^{bb})₂, —C(═NR^{bb})R^{aa}, —C(═NR^{bb})OR^{aa}, —OC(═NR^{bb})R^{aa}—, —OC(═NR^{bb})OR^{aa}—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of 19F with 18F, replacement of a carbon by a 13C- or 14C-enriched carbon, and/or replacement of an oxygen atom with 18O, are within the scope of the disclosure. Other examples of isotopes include 15N, 18O, 17O, 31P, 32P, 35S, 18F, 36Cl and 123I. Compounds with such isotopically enriched atoms are useful, for example, as analytical tools or probes in biological assays.

Certain isotopically-labelled compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability.

Certain isotopically-labelled compounds of Formula (I) can be useful for medical imaging purposes, for example, those labeled with positron-emitting isotopes like 11C or 18F can be useful for application in Positron Emission Tomography (PET) and those labeled with gamma ray emitting isotopes like 123I can be useful for application in Single Photon Emission Computed Tomography (SPECT). Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., 2H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t½>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

TABLE 1
In the various embodiments described herein, the Dengue inhibitors of any one of
Formulae I-V or pharmaceutically acceptable salt and/or stereoisomer thereof, is one selected
from the Compounds in Table 1 shown below.

| Example | | |
|---|---|---|
| # | Structure | Nomenclature |
| 1 | 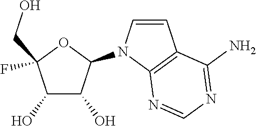 [SMILES: Nc1ncnc2c1ccn2[C@@H]1O[C@](F)(CO)[C@@H](O)[C@H]1O] | (2S,3S,4R,5R)-5-(4-amino- 7H-pyrrolo[2,3-d]pyrimidin-7-yl)- 2-fluoro-2- (hydroxymethyl)tetrahydrofuran- 3,4-diol |
| 2 | 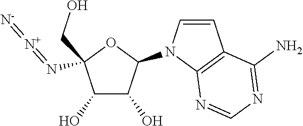 [SMILES: [N-]=[N+]=N[C@]1(CO)O[C@@H](n2ccc3c(N)ncnc32)[C@H](O)[C@@H]1O] | (2R,3S,4R,5R)-5-(4-amino- 7H-pyrrolo[2,3-d]pyrimidin-7-yl)- 2-azido-2- (hydroxymethyl)tetrahydrofuran- 3,4-diol |
| 3 | 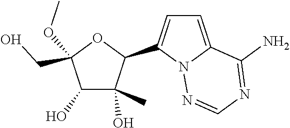 [SMILES: CO[C@]1(CO)O[C@@H](c2ccc3c(N)ncnn23)[C@](C)(O)[C@@H]1O] | (2R,3S,4R,5S)-5-(4- aminopyrrolo[2,1-f][1,2,4]triazin- 7-yl)-2-(hydroxymethyl)-2- methoxy-4-methyltetrahydrofuran- 3,4-diol |
| 4 | 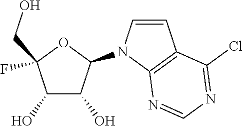 [SMILES: OC[C@@]1(F)O[C@@H](n2ccc3c(Cl)ncnc32)[C@H](O)[C@@H]1O] | (2S,3S,4R,5R)-5-(4-chloro- 7H-pyrrolo[2,3-d]pyrimidin-7-yl)- 2-fluoro-2- (hydroxymethyl)tetrahydrofuran- 3,4-diol |
| 5 | 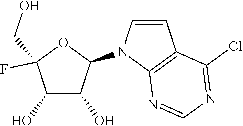 [SMILES: OC[C@]1(F)O[C@@H](n2ccc3c(Cl)ncnc32)[C@H](O)[C@@H]1O] | (2R,3S,4R,5R)-5-(4-chloro- 7H-pyrrolo[2,3-d]pyrimidin-7-yl)- 2-fluoro-2- (hydroxymethyl)tetrahydrofuran- 3,4-diol |
| 6 | 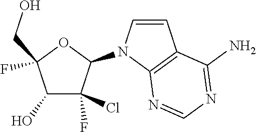 [SMILES: Nc1ncnc2c1ccn2[C@@H]1O[C@](F)(CO)[C@@H](O)[C@]1(F)Cl] | (2S,3R,4S,5R)-5-(4-amino- 7H-pyrrolo[2,3-d]pyrimidin-7-yl)- 4-chloro-2,4-difluoro-2- (hydroxymethyl)tetrahydrofuran- 3-ol |
| 7 | 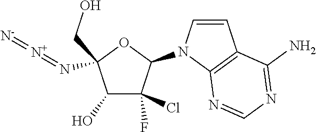 [SMILES: [N-]=[N+]=N[C@]1(CO)O[C@@H](n2ccc3c(N)ncnc32)[C@@](F)(Cl)[C@@H]1O] | (2R,3R,4S,5R)-5-(4-amino- 7H-pyrrolo[2,3-d]pyrimidin-7-yl)- 2-azido-4-chloro-4-fluoro-2- (hydroxymethyl)tetrahydrofuran- 3-ol |
| 8 | 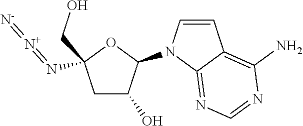 [SMILES: [N-]=[N+]=N[C@@]1(CO)C[C@@H](O)[C@H](n2ccc3c(N)ncnc32)O1] | (2R,3R,5R)-2-(4-amino-7H- pyrrolo[2,3-d]pyrimidin-7-yl)-5- azido-5- (hydroxymethyl)tetrahydrofuran- 3-ol |
| 9 | 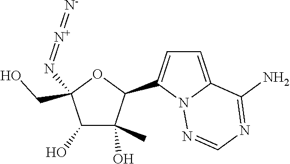 [SMILES: C[C@]1(O)[C@H](c2ccc3c(N)ncnn23)O[C@@](CO)(N=[N+]=[N-])[C@H]1O] | (2R,3S,4R,5S)-5-(4- aminopyrrolo[2,1-f][1,2,4]triazin- 7-yl)-2-azido-2-(hydroxymethyl)- 4-methyltetrahydrofuran-3,4-diol |
| 10 | 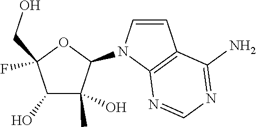 [SMILES: C[C@]1(O)[C@H](n2ccc3c(N)ncnc32)O[C@](F)(CO)[C@H]1O] | (2S,3S,4R,5R)-5-(4-amino- 7H-pyrrolo[2,3-d]pyrimidin-7-yl)- 2-fluoro-2-(hydroxymethyl)-4- methyltetrahydrofuran-3,4-diol |
| 11 | 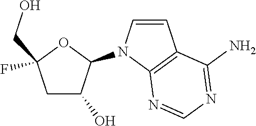 [SMILES: Nc1ncnc2c1ccn2[C@@H]1O[C@](F)(CO)C[C@H]1O] | (2R,3R,5R)-2-(4-amino-7H- pyrrolo[2,3-d]pyrimidin-7-yl)-5- fluoro-5- (hydroxymethyl)tetrahydrofuran- 3-ol |
| 12 | 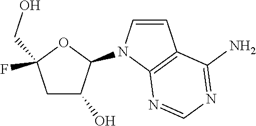 [SMILES: Nc1ncnc2c1ccn2[C@@H]1O[C@@](F)(CO)C[C@H]1O] | (2R,3R,5S)-2-(4-amino-7H- pyrrolo[2,3-d]pyrimidin-7-yl)-5- fluoro-5- (hydroxymethyl)tetrahydrofuran- 3-ol |
| 13 | 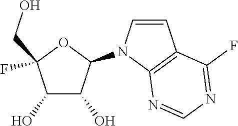 [SMILES: OC[C@@]1(F)O[C@@H](n2ccc3c(F)ncnc32)[C@H](O)[C@@H]1O] | (2S,3S,4R,5R)-2-fluoro-5-(4- fluoro-7H-pyrrolo[2,3- d]pyrimidin-7-yl)-2- (hydroxymethyl)tetrahydrofuran- 3,4-diol |
| 14 | 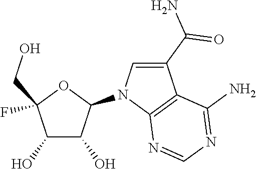 [SMILES: NC(=O)c1cn([C@@H]2O[C@](F)(CO)[C@@H](O)[C@H]2O)c2ncnc(N)c12] | 4-amino-7-((2R,3R,4S,5S)-5- fluoro-3,4-dihydroxy-5- (hydroxymethyl)tetrahydrofuran- 2-yl)-7H-pyrrolo[2,3- d]pyrimidine-5-carboxamide |
| 15 | 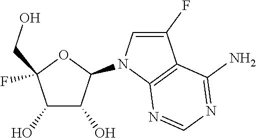 [SMILES: Nc1ncnc2c1c(F)cn2[C@@H]1O[C@](F)(CO)[C@@H](O)[C@H]1O] | (2S,3S,4R,5R)-5-(4-amino-5- fluoro-7H-pyrrolo[2,3- d]pyrimidin-7-yl)-2-fluoro-2- (hydroxymethyl)tetrahydrofuran- 3,4-diol |
| 16 | 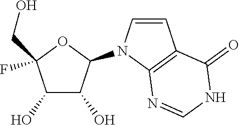 [SMILES: O=c1[nH]cnc2c1ccn2[C@@H]1O[C@](F)(CO)[C@@H](O)[C@H]1O] | 7-((2R,3R,4S,5S)-5-fluoro- 3,4-dihydroxy-5- (hydroxymethyl)tetrahydrofuran- 2-yl)-3,7-dihydro-4H-pyrrolo[2,3- d]pyrimidin-4-one |
| 17 | 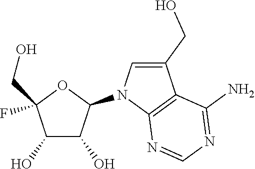 [SMILES: Nc1ncnc2c1c(CO)cn2[C@@H]1O[C@](F)(CO)[C@@H](O)[C@H]1O] | (2S,3S,4R,5R)-5-(4-amino-5- (hydroxymethyl)-7H-pyrrolo[2,3- d]pyrimidin-7-yl)-2-fluoro-2- (hydroxymethyl)tetrahydrofuran- 3,4-diol |
| 18 | 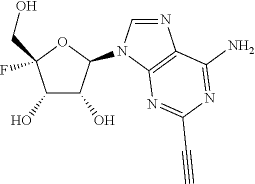 [SMILES: C#Cc1nc(N)c2ncn([C@@H]3O[C@](F)(CO)[C@@H](O)[C@H]3O)c2n1] | (2S,3S,4R,5R)-5-(6-amino-2- ethynyl-9H-purin-9-yl)-2-fluoro-2- (hydroxymethyl)tetrahydrofuran- 3,4-diol |
| 19 | 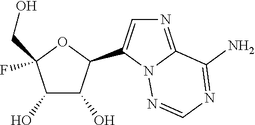 [SMILES: Nc1ncnn2c([C@@H]3O[C@](F)(CO)[C@@H](O)[C@H]3O)cnc12] | (2S,3S,4R,5S)-5-(4- aminoimidazo[2,1-f][1,2,4]triazin- 7-yl)-2-fluoro-2- (hydroxymethyl)tetrahydrofuran- 3,4-diol |
| 20 | 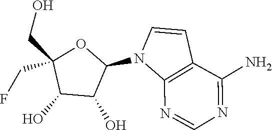 [SMILES: Nc1ncnc2c1ccn2[C@@H]1O[C@@](CO)(CF)[C@@H](O)[C@H]1O] | (2R,3S,4R,5R)-5-(4-amino- 7H-pyrrolo[2,3-d]pyrimidin-7-yl)- 2-(fluoromethyl)-2- (hydroxymethyl)tetrahydrofuran- 3,4-diol |
| 21 | 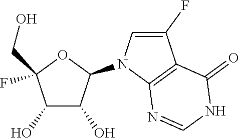 [SMILES: O=c1[nH]cnc2c1c(F)cn2[C@@H]1O[C@](F)(CO)[C@@H](O)[C@H]1O] | 5-fluoro-7-((2R,3R,4S,5S)-5- fluoro-3,4-dihydroxy-5- (hydroxymethyl)tetrahydrofuran- 2-yl)-3,7-dihydro-4H-pyrrolo[2,3- d]pyrimidin-4-one |
| 22 | 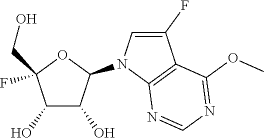 [SMILES: COc1ncnc2c1c(F)cn2[C@@H]1O[C@](F)(CO)[C@@H](O)[C@H]1O] | (2S,3S,4R,5R)-2-fluoro-5-(5- fluoro-4-methoxy-7H-pyrrolo[2,3- d]pyrimidin-7-yl)-2- (hydroxymethyl)tetrahydrofuran- 3,4-diol |
| 23 | 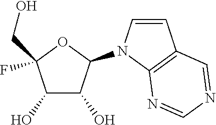 [SMILES: OC[C@@]1(F)O[C@@H](n2ccc3cncnc32)[C@H](O)[C@@H]1O] | (2S,3S,4R,5R)-2-fluoro-2- (hydroxymethyl)-5-(7H- pyrrolo[2,3-d]pyrimidin-7- yl)tetrahydrofuran-3,4-diol |
| 24 | 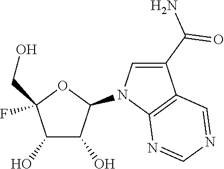 [SMILES: NC(=O)c1cn([C@@H]2O[C@](F)(CO)[C@@H](O)[C@H]2O)c2ncncc12] | 7-((2R,3R,4S,5S)-5-fluoro- 3,4-dihydroxy-5- (hydroxymethyl)tetrahydrofuran- 2-yl)-7H-pyrrolo[2,3- d]pyrimidine-5-carboxamide |
| 25 | 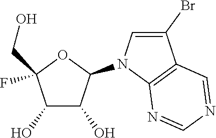 [SMILES: OC[C@@]1(F)O[C@@H](n2cc(Br)c3cncnc32)[C@H](O)[C@@H]1O] | (2S,3S,4R,5R)-5-(5-bromo- 7H-pyrrolo[2,3-d]pyrimidin-7-yl)- 2-fluoro-2- (hydroxymethyl)tetrahydrofuran- 3,4-diol |
| 26 | 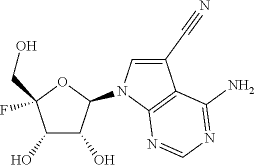 [SMILES: N#Cc1cn([C@@H]2O[C@](F)(CO)[C@@H](O)[C@H]2O)c2ncnc(N)c12] | 4-amino-7-((2R,3R,4S,5S)-5- fluoro-3,4-dihydroxy-5- (hydroxymethyl)tetrahydrofuran- 2-yl)-7H-pyrrolo[2,3- d]pyrimidine-5-carbonitrile |
| 27 | 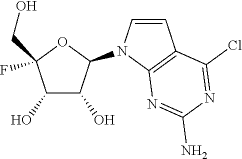 [SMILES: Nc1nc(Cl)c2ccn([C@@H]3O[C@](F)(CO)[C@@H](O)[C@H]3O)c2n1] | (2S,3S,4R,5R)-5-(2-amino-4- chloro-7H-pyrrolo[2,3- d]pyrimidin-7-yl)-2-fluoro-2- (hydroxymethyl)tetrahydrofuran- 3,4-diol |
| 28 | 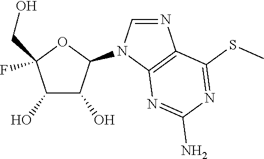 [SMILES: CSc1nc(N)nc2c1ncn2[C@@H]1O[C@](F)(CO)[C@@H](O)[C@H]1O] | (2S,3S,4R,5R)-2-fluoro-2- (hydroxymethyl)-5-(6- (methylthio)-9H-purin-9- yl)tetrahydrofuran-3,4-diol |
| 29 | 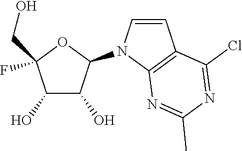 [SMILES: Cc1nc(Cl)c2ccn([C@@H]3O[C@](F)(CO)[C@@H](O)[C@H]3O)c2n1] | (2S,3S,4R,5R)-5-(4-chloro-2- methyl-7H-pyrrolo[2,3- d]pyrimidin-7-yl)-2-fluoro-2- (hydroxymethyl)tetrahydrofuran- 3,4-diol |
| 30 | 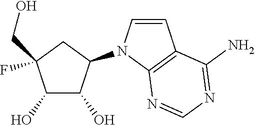 [SMILES: Nc1ncnc2c1ccn2[C@@H]1C[C@](F)(CO)[C@@H](O)[C@H]1O] | (1S,2S,3S,5R)-5-(4-amino-7H- pyrrolo[2,3-d]pyrimidin-7-yl)-3- fluoro-3- (hydroxymethyl)cyclopentane-1,2- diol |
| 31 | 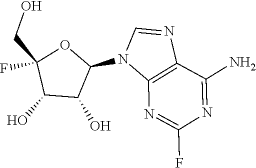 [SMILES: Nc1nc(F)nc2c1ncn2[C@@H]1O[C@](F)(CO)[C@@H](O)[C@H]1O] | (2S,3S,4R,5R)-5-(6-amino-2- fluoro-9H-purin-9-yl)-2-fluoro-2- (hydroxymethyl)tetrahydrofuran- 3,4-diol |
| 32 | 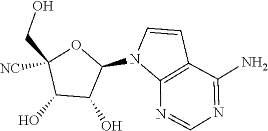 [SMILES: N#C[C@]1(CO)O[C@@H](n2ccc3c(N)ncnc32)[C@H](O)[C@@H]1O] | (3S,4R,5R)-5-(4-amino-7H- pyrrolo[2,3-d]pyrimidin-7-yl)-3,4- dihydroxy-2- (hydroxymethyl)tetrahydrofuran- 2-carbonitrile |
| 33 | 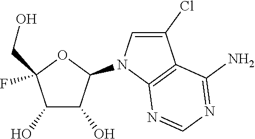 [SMILES: Nc1ncnc2c1c(Cl)cn2[C@@H]1O[C@](F)(CO)[C@@H](O)[C@H]1O] | (2S,3S,4R,5R)-5-(4-amino-5- chloro-7H-pyrrolo[2,3- d]pyrimidin-7-yl)-2-fluoro-2- (hydroxymethyl)tetrahydrofuran- 3,4-diol |
| 34 | 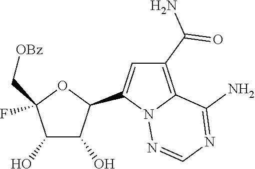 | ((2S,3S,4R,5S)-5-(4-amino-5- carbamoylpyrrolo[2,1- f][1,2,4]triazin-7-yl)-2-fluoro-3,4- dihydroxytetrahydrofuran-2- yl)methyl benzoate |
| 35 | 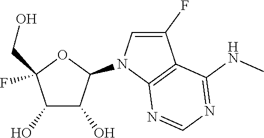 [SMILES: CNc1ncnc2c1c(F)cn2[C@@H]1O[C@](F)(CO)[C@@H](O)[C@H]1O] | (2S,3S,4R,5R)-2-fluoro-5-(5- fluoro-4-(methylamino)-7H- pyrrolo[2,3-d]pyrimidin-7-yl)-2- (hydroxymethyl)tetrahydrofuran- 3,4-diol |
| 36 | 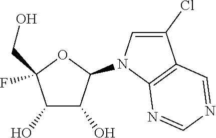 [SMILES: OC[C@@]1(F)O[C@@H](n2cc(Cl)c3cncnc32)[C@H](O)[C@@H]1O] | (2S,3S,4R,5R)-5-(5-chloro- 7H-pyrrolo[2,3-d]pyrimidin-7-yl)- 2-fluoro-2- (hydroxymethyl)tetrahydrofuran- 3,4-diol |
| 37 | 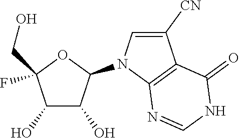 [SMILES: N#Cc1cn([C@@H]2O[C@](F)(CO)[C@@H](O)[C@H]2O)c2nc[nH]c(=O)c12] | 7-((2R,3R,4S,5S)-5-fluoro- 3,4-dihydroxy-5- (hydroxymethyl)tetrahydrofuran- 2-yl)-4-oxo-4,7-dihydro-3H- pyrrolo[2,3-d]pyrimidine-5- carbonitrile |
| 38 | 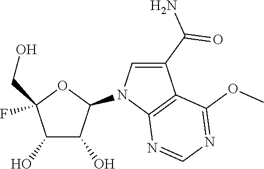 [SMILES: COc1ncnc2c1c(C(N)=O)cn2[C@@H]1O[C@](F)(CO)[C@@H](O)[C@H]1O] | 7-((2R,3R,4S,5S)-5-fluoro- 3,4-dihydroxy-5- (hydroxymethyl)tetrahydrofuran- 2-yl)-4-methoxy-7H-pyrrolo[2,3- d]pyrimidine-5-carboxamide |
| 39 | 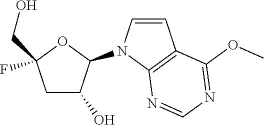 [SMILES: COc1ncnc2c1ccn2[C@@H]1O[C@](F)(CO)C[C@H]1O] | (2R,3R,5R)-5-fluoro-5- (hydroxymethyl)-2-(4-methoxy- 7H-pyrrolo[2,3-d]pyrimidin-7- yl)tetrahydrofuran-3-ol |
| 40 | 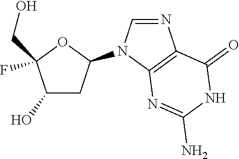 [SMILES: Nc1nc2c(ncn2[C@H]2C[C@H](O)[C@@](F)(CO)O2)c(=O)[nH]1] | 2-amino-9-((2R,4S,5S)-5- fluoro-4-hydroxy-5- (hydroxymethyl)tetrahydrofuran- 2-yl)-1,9-dihydro-6H-purin-6-one |
| 41 | 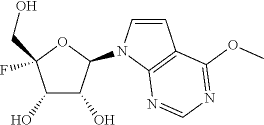 [SMILES: COc1ncnc2c1ccn2[C@@H]1O[C@](F)(CO)[C@@H](O)[C@H]1O] | (2S,3S,4R,5R)-2-fluoro-2- (hydroxymethyl)-5-(4-methoxy- 7H-pyrrolo[2,3-d]pyrimidin-7- yl)tetrahydrofuran-3,4-diol |
| 42 | 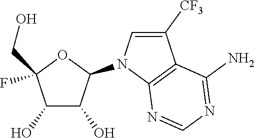 [SMILES: Nc1ncnc2c1c(C(F)(F)F)cn2[C@@H]1O[C@](F)(CO)[C@@H](O)[C@H]1O] | (2S,3S,4R,5R)-5-(4-amino-5- (trifluoromethyl)-7H-pyrrolo[2,3- d]pyrimidin-7-yl)-2-fluoro-2- (hydroxymethyl)tetrahydrofuran- 3,4-diol |
| 43 | 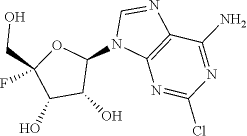 [SMILES: Nc1nc(Cl)nc2c1ncn2[C@@H]1O[C@](F)(CO)[C@@H](O)[C@H]1O] | (2S,3S,4R,5R)-5-(6-amino-2- chloro-9H-purin-9-yl)-2-fluoro-2- (hydroxymethyl)tetrahydrofuran- 3,4-diol |
| 44 | 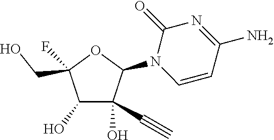 [SMILES: C#C[C@]1(O)[C@H](n2ccc(N)nc2=O)O[C@](F)(CO)[C@H]1O] | 4-amino-1-((2R,3R,4S,5S)-3- ethynyl-5-fluoro-3,4-dihydroxy-5- (hydroxymethyl)tetrahydrofuran- 2-yl)pyrimidin-2(1H)-one |
| 45 | 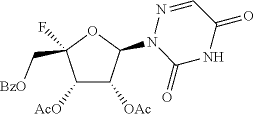 | (2S,3S,4R,5R)-2- ((benzoyloxy)methyl)-5-(3,5- dioxo-4,5-dihydro-1,2,4-triazin- 2(3H)-yl)-2-fluorotetrahydrofuran- 3,4-diyl diacetate |
| 46 | 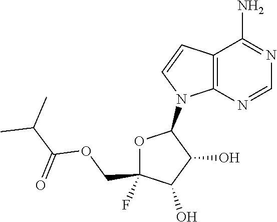 [SMILES: CC(C)C(=O)OC[C@@]1(F)O[C@@H](n2ccc3c(N)ncnc32)[C@H](O)[C@@H]1O] | ((2S,3S,4R,5R)-5-(4-amino- 7H-pyrrolo[2,3-d]pyrimidin-7-yl)- 2-fluoro-3,4- dihydroxytetrahydrofuran-2- yl)methyl isobutyrate |
| 47 | 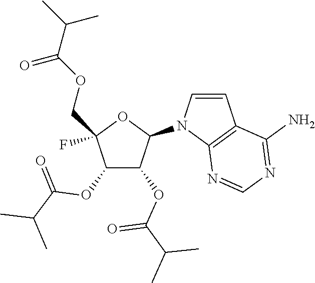 [SMILES: CC(C)C(=O)OC[C@@]1(F)O[C@@H](n2ccc3c(N)ncnc32)[C@H](OC(=O)C(C)C)[C@@H]1OC(=O)C(C)C] | (2S,3S,4R,5R)-5-(4-amino- 7H-pyrrolo[2,3-d]pyrimidin-7-yl)- 2-fluoro-2- ((isobutyryloxy)methyl) tetrahydrofuran-3,4-diyl bis(2- methylpropanoate) |
| 48 | 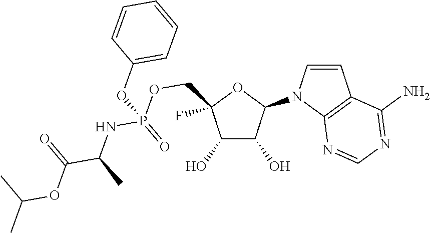 [SMILES: CC(C)OC(=O)[C@H](C)N[P@](=O)(OC[C@@]1(F)O[C@@H](n2ccc3c(N)ncnc32)[C@H](O)[C@@H]1O)Oc1ccccc1] | isopropyl ((S)- (((2S,3S,4R,5R)-5-(4-amino-7H- pyrrolo[2,3-d]pyrimidin-7-yl)-2- fluoro-3,4- dihydroxytetrahydrofuran-2- yl)methoxy)(phenoxy)phosphoryl)- L-alaninate |
| 49 | 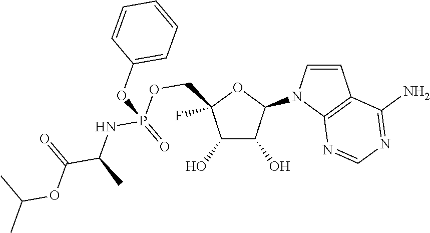 [SMILES: CC(C)OC(=O)[C@H](C)N[P@@](=O)(OC[C@@]1(F)O[C@@H](n2ccc3c(N)ncnc32)[C@H](O)[C@@H]1O)Oc1ccccc1] | isopropyl ((R)- (((2S,3S,4R,5R)-5-(4-amino-7H- pyrrolo[2,3-d]pyrimidin-7-yl)-2- fluoro-3,4- dihydroxytetrahydrofuran-2- yl)methoxy)(phenoxy)phosphoryl)- L-alaninate |
| 50 | 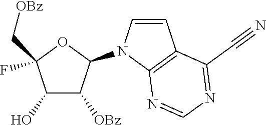 | ((2S,3S,4R,5R)-4- (benzoyloxy)-5-(4-cyano-7H- pyrrolo[2,3-d]pyrimidin-7-yl)-2- fluoro-3-hydroxytetrahydrofuran- 2-yl)methyl benzoate |
| 51 | 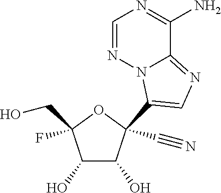 [SMILES: N#C[C@@]1(c2cnc3c(N)ncnn23)O[C@](F)(CO)[C@@H](O)[C@H]1O] | (2R,3R,4S,5S)-2-(4- aminoimidazo[2,1-f][1,2,4]triazin- 7-yl)-5-fluoro-3,4-dihydroxy-5- (hydroxymethyl)tetrahydrofuran- 2-carbonitrile |
| 52 | 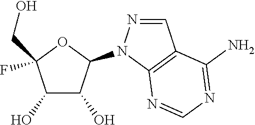 [SMILES: Nc1ncnc2c1cnn2[C@@H]1O[C@](F)(CO)[C@@H](O)[C@H]1O] | (2S,3S,4R,5R)-5-(4-amino- 1H-pyrazolo[3,4-d]pyrimidin-1- yl)-2-fluoro-2- (hydroxymethyl)tetrahydrofuran- 3,4-diol |
| 53 | 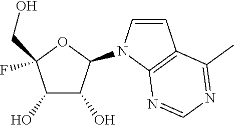 [SMILES: Cc1ncnc2c1ccn2[C@@H]1O[C@](F)(CO)[C@@H](O)[C@H]1O] | (2S,3S,4R,5R)-2-fluoro-2- (hydroxymethyl)-5-(4-methyl-7H- pyrrolo[2,3-d]pyrimidin-7- yl)tetrahydrofuran-3,4-diol |
| 54 | 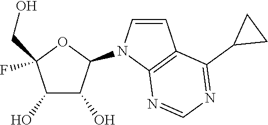 [SMILES: OC[C@@]1(F)O[C@@H](n2ccc3c(C4CC4)ncnc32)[C@H](O)[C@@H]1O] | (2S,3S,4R,5R)-5-(4- cyclopropyl-7H-pyrrolo[2,3- d]pyrimidin-7-yl)-2-fluoro-2- (hydroxymethyl)tetrahydrofuran- 3,4-diol |
| 55 | 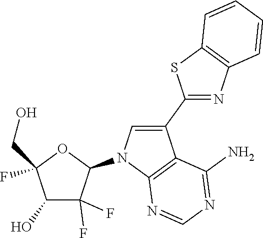 [SMILES: Nc1ncnc2c1c(-c1nc3ccccc3s1)cn2[C@@H]1O[C@](F)(CO)[C@@H](O)C1(F)F] | (2S,3R,5R)-5-(4-amino-5- (benzo[d]thiazol-2-yl)-7H- pyrrolo[2,3-d]pyrimidin-7-yl)- 2,4,4-trifluoro-2- (hydroxymethyl)tetrahydrofuran- 3-ol |
| 56 | 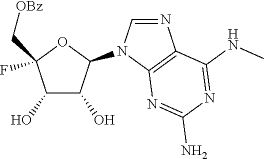 | ((2S,3S,4R,5R)-5-(2-amino-4- (methylamino)-7H-pyrrolo[2,3- d]pyrimidin-7-yl)-2-fluoro-3,4- dihydroxytetrahydrofuran-2- yl)methyl benzoate |
| 57 | 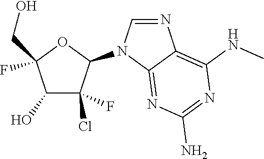 [SMILES: CNc1nc(N)nc2c1ncn2[C@@H]1O[C@](F)(CO)[C@@H](O)[C@]1(F)Cl] | (2S,3R,4S,5R)-5-(2-amino-6- (methylamino)-9H-purin-9-yl)-4- chloro-2,4-difluoro-2- (hydroxymethyl)tetrahydrofuran- 3-ol |
| 58 | 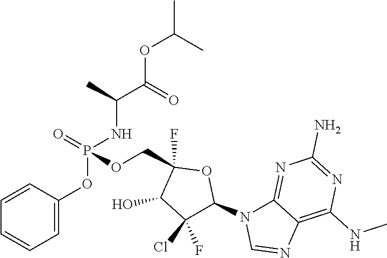 [SMILES: CNc1nc(N)nc2c1ncn2[C@@H]1O[C@](F)(CO[P@@](=O)(N[C@@H](C)C(=O)OC(C)C)Oc2ccccc2)[C@@H](O)[C@]1(F)Cl] | isopropyl ((R)- (((2S,3R,4S,5R)-5-(2-amino-6- (methylamino)-9H-purin-9-yl)-4- chloro-2,4-difluoro-3- hydroxytetrahydrofuran-2- yl)methoxy)(phenoxy)phosphoryl)- L-alaninate |
| 59 | 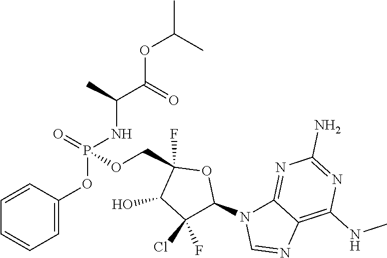 [SMILES: CNc1nc(N)nc2c1ncn2[C@@H]1O[C@](F)(CO[P@](=O)(N[C@@H](C)C(=O)OC(C)C)Oc2ccccc2)[C@@H](O)[C@]1(F)Cl] | isopropyl ((S)- (((2S,3R,4S,5R)-5-(2-amino-6- (methylamino)-9H-purin-9-yl)-4- chloro-2,4-difluoro-3- hydroxytetrahydrofuran-2- yl)methoxy)(phenoxy)phosphoryl)- L-alaninate |
| 60 | 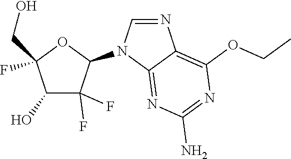 [SMILES: CCOc1nc(N)nc2c1ncn2[C@@H]1O[C@](F)(CO)[C@@H](O)C1(F)F] | (2S,3R,5R)-5-(2-amino-6- ethoxy-9H-purin-9-yl)-2,4,4- trifluoro-2- (hydroxymethyl)tetrahydrofuran- 3-ol |
| 61 | 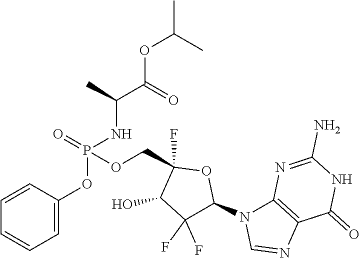 [SMILES: CC(C)OC(=O)[C@H](C)NP(=O)(OC[C@@]1(F)O[C@@H](n2cnc3c(=O)[nH]c(N)nc32)C(F)(F)[C@@H]1O)Oc1ccccc1] | isopropyl ((((2S,3R,5R)-5-(2- amino-6-oxo-1,6-dihydro-9H- purin-9-yl)-2,4,4-trifluoro-3- hydroxytetrahydrofuran-2- yl)methoxy)(phenoxy)phosphoryl)- L-alaninate |
| 62 | 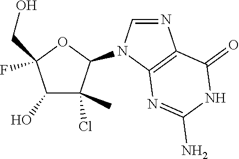 [SMILES: C[C@]1(Cl)[C@H](n2cnc3c(=O)[nH]c(N)nc32)O[C@](F)(CO)[C@H]1O] | 2-amino-9-((2R,3R,4R,5S)-3- chloro-5-fluoro-4-hydroxy-5- (hydroxymethyl)-3- methyltetrahydrofuran-2-yl)-1,9- dihydro-6H-purin-6-one |
| 63 | 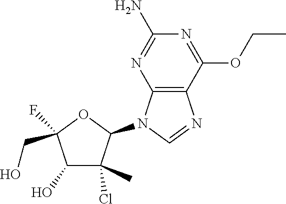 [SMILES: CCOc1nc(N)nc2c1ncn2[C@@H]1O[C@](F)(CO)[C@@H](O)[C@@]1(C)Cl] | (2S,3R,4R,5R)-5-(2-amino-6- ethoxy-9H-purin-9-yl)-4-chloro-2- fluoro-2-(hydroxymethyl)-4- methyltetrahydrofuran-3-ol |
| 64 | 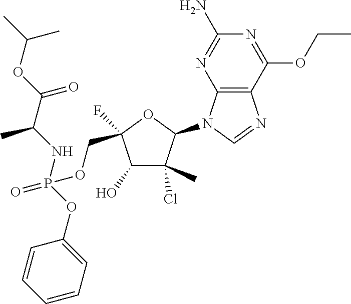 [SMILES: CCOc1nc(N)nc2c1ncn2[C@@H]1O[C@](F)(COP(=O)(N[C@@H](C)C(=O)OC(C)C)Oc2ccccc2)[C@@H](O)[C@@]1(C)Cl] | isopropyl ((((2S,3R,4R,5R)-5- (2-amino-6-ethoxy-9H-purin-9- yl)-4-chloro-2-fluoro-3-hydroxy-4- methyltetrahydrofuran-2- yl)methoxy)(phenoxy)phosphoryl)- L-alaninate |
| 65 | 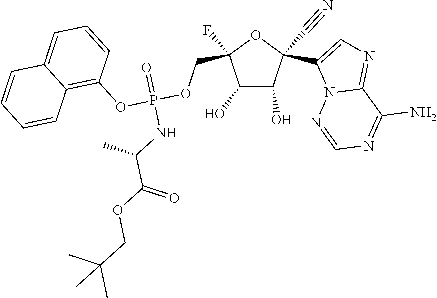 [SMILES: C[C@H](NP(=O)(OC[C@@]1(F)O[C@@](C#N)(c2cnc3c(N)ncnn23)[C@H](O)[C@@H]1O)Oc1cccc2ccccc12)C(=O)OCC(C)(C)C] | neopentyl ((((2S,3S,4R,5R)-5- (4-aminoimidazo[2,1- f][1,2,4]triazin-7-yl)-5-cyano-2- fluoro-3,4- dihydroxytetrahydrofuran-2- yl)methoxy)(naphthalen-1- yloxy)phosphoryl)-L-alaninate |
| 66 | 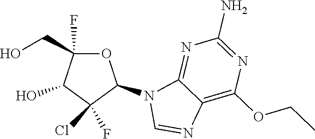 [SMILES: CCOc1nc(N)nc2c1ncn2[C@@H]1O[C@](F)(CO)[C@@H](O)[C@]1(F)Cl] | (2S,3R,4S,5R)-5-(2-amino-6- ethoxy-9H-purin-9-yl)-4-chloro- 2,4-difluoro-2- (hydroxymethyl)tetrahydrofuran- 3-ol |
| 67 | 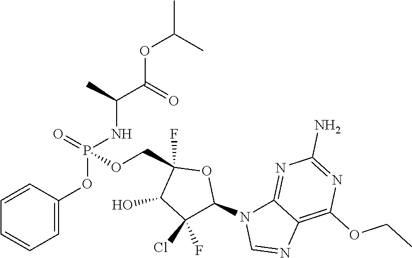 [SMILES: CCOc1nc(N)nc2c1ncn2[C@@H]1O[C@](F)(CO[P@](=O)(N[C@@H](C)C(=O)OC(C)C)Oc2ccccc2)[C@@H](O)[C@]1(F)Cl] | isopropyl ((((2S,3R,4S,5R)-5- (2-amino-6-ethoxy-9H-purin-9- yl)-4-chloro-2,4-difluoro-3- hydroxytetrahydrofuran-2- yl)methoxy)(phenoxy)phosphoryl)- L-alaninate |
| 68 | 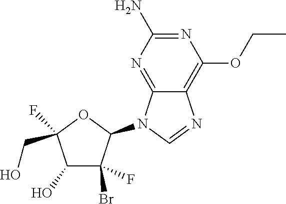 [SMILES: CCOc1nc(N)nc2c1ncn2[C@@H]1O[C@](F)(CO)[C@@H](O)[C@]1(F)Br] | (2S,3R,4S,5R)-5-(2-amino-6- ethoxy-9H-purin-9-yl)-4-bromo- 2,4-difluoro-2- (hydroxymethyl)tetrahydrofuran- 3-ol |
| 69 | 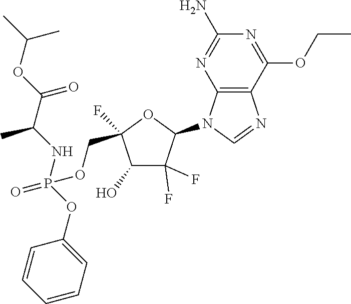 [SMILES: CCOc1nc(N)nc2c1ncn2[C@@H]1O[C@](F)(COP(=O)(N[C@@H](C)C(=O)OC(C)C)Oc2ccccc2)[C@@H](O)C1(F)F] | isopropyl ((((2S,3R,5R)-5-(2- amino-6-ethoxy-9H-purin-9-yl)- 2,4,4-trifluoro-3- hydroxytetrahydrofuran-2- yl)methoxy)(phenoxy)phosphoryl)- L-alaninate |
| 70 | 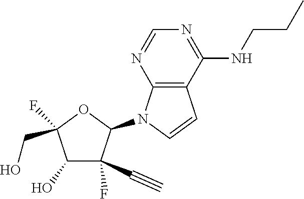 [SMILES: C#C[C@]1(F)[C@H](n2ccc3c(NCCC)ncnc32)O[C@](F)(CO)[C@H]1O] | (2S,3S,4R,5R)-4-ethynyl-2- fluoro-2-(hydroxymethyl)-5-(4- (propylamino)-7H-pyrrolo[2,3- d]pyrimidin-7-yl)tetrahydrofuran- 3,4-diol |
| 71 | 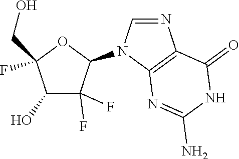 [SMILES: Nc1nc2c(ncn2[C@@H]2O[C@](F)(CO)[C@@H](O)C2(F)F)c(=O)[nH]1] | 2-amino-9-((2R,4R,5S)-3,3,5- trifluoro-4-hydroxy-5- (hydroxymethyl)tetrahydrofuran- 2-yl)-1,9-dihydro-6H-purin-6-one |
| 72 | 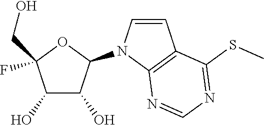 [SMILES: CSc1ncnc2c1ccn2[C@@H]1O[C@](F)(CO)[C@@H](O)[C@H]1O] | (2S,3S,4R,5R)-2-fluoro-2- (hydroxymethyl)-5-(4- (methylthio)-7H-pyrrolo[2,3- d]pyrimidin-7-yl)tetrahydrofuran- 3,4-diol |
| 73 | 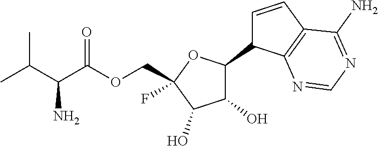 [SMILES: CC(C)[C@H](N)C(=O)OC[C@@]1(F)O[C@@H](C2C=Cc3c(N)ncnc32)[C@H](O)[C@@H]1O] | ((2S,3S,4R,5R)-5-(4-amino- 7H-pyrrolo[2,3-d]pyrimidin-7-yl)- 2-fluoro-3,4- dihydroxytetrahydrofuran-2- yl)methyl L-valinate |
| 74 | 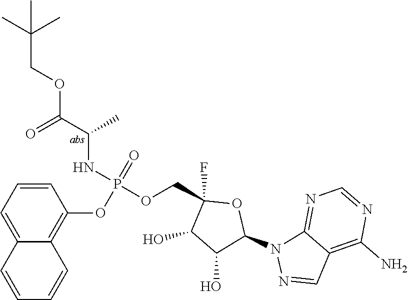 [SMILES: C[C@H](NP(=O)(OC[C@@]1(F)O[C@@H](n2ncc3c(N)ncnc32)[C@H](O)[C@@H]1O)Oc1cccc2ccccc12)C(=O)OCC(C)(C)C] | neopentyl ((((2S,3S,4R,5R)-5- (4-amino-1H-pyrazolo[3,4- d]pyrimidin-1-yl)-2-fluoro-3,4- dihydroxytetrahydrofuran-2- yl)methoxy)(naphthalen-1- yloxy)phosphoryl)-L-alaninate |
| 75 | 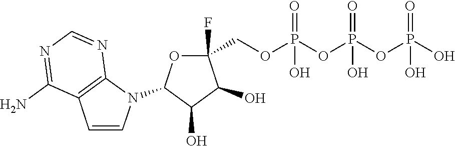 [SMILES: Nc1ncnc2c1ccn2[C@@H]1O[C@](F)(COP(=O)(O)OP(=O)(O)OP(=O)(O)O)[C@@H](O)[C@H]1O] | ((2S,3S,4R,5R)-5-(4-amino- 7H-pyrrolo[2,3-d]pyrimidin-7-yl)- 2-fluoro-3,4- dihydroxytetrahydrofuran-2- yl)methyl tetrahydrogen triphosphate |
| 76 | 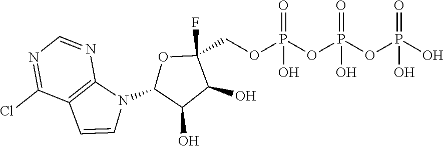 [SMILES: O=P(O)(O)OP(=O)(O)OP(=O)(O)OC[C@@]1(F)O[C@@H](n2ccc3c(Cl)ncnc32)[C@H](O)[C@@H]1O] | ((2S,3S,4R,5R)-5-(4-chloro- 7H-pyrrolo[2,3-d]pyrimidin-7-yl)- 2-fluoro-3,4- dihydroxytetrahydrofuran-2- yl)methyl tetrahydrogen triphosphate |
| 77 | 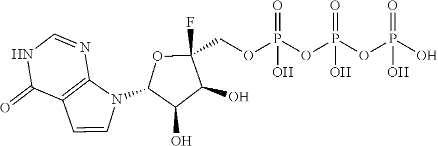 [SMILES: O=c1[nH]cnc2c1ccn2[C@@H]1O[C@](F)(COP(=O)(O)OP(=O)(O)OP(=O)(O)O)[C@@H](O)[C@H]1O] | ((2S,3S,4R,5R)-2-fluoro-3,4- dihydroxy-5-(4-oxo-3,4-dihydro- 7H-pyrrolo[2,3-d]pyrimidin-7- yl)tetrahydrofuran-2-yl)methyl tetrahydrogen triphosphate |
| 78 | 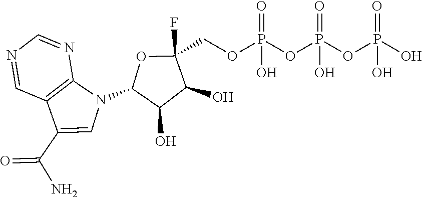 [SMILES: NC(=O)c1cn([C@@H]2O[C@](F)(COP(=O)(O)OP(=O)(O)OP(=O)(O)O)[C@@H](O)[C@H]2O)c2ncncc12] | ((2S,3S,4R,5R)-5-(5- carbamoyl-7H-pyrrolo[2,3- d]pyrimidin-7-yl)-2-fluoro-3,4- dihydroxytetrahydrofuran-2- yl)methyl tetrahydrogen triphosphate |
| 79 | 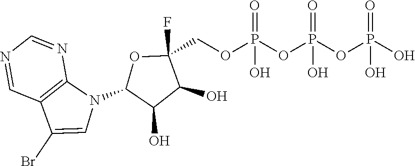 [SMILES: O=P(O)(O)OP(=O)(O)OP(=O)(O)OC[C@@]1(F)O[C@@H](n2cc(Br)c3cncnc32)[C@H](O)[C@@H]1O] | ((2S,3S,4R,5R)-5-(5-bromo- 7H-pyrrolo[2,3-d]pyrimidin-7-yl)- 2-fluoro-3,4- dihydroxytetrahydrofuran-2- yl)methyl tetrahydrogen triphosphate |
| 80 | 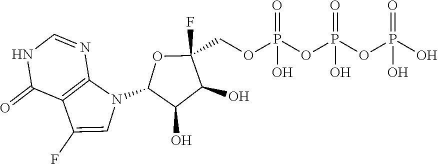 [SMILES: O=c1[nH]cnc2c1c(F)cn2[C@@H]1O[C@](F)(COP(=O)(O)OP(=O)(O)OP(=O)(O)O)[C@@H](O)[C@H]1O] | ((2S,3S,4R,5R)-2-fluoro-5-(5- fluoro-4-oxo-3,4-dihydro-7H- pyrrolo[2,3-d]pyrimidin-7-yl)-3,4- dihydroxytetrahydrofuran-2- yl)methyl tetrahydrogen triphosphate |
| 81 | 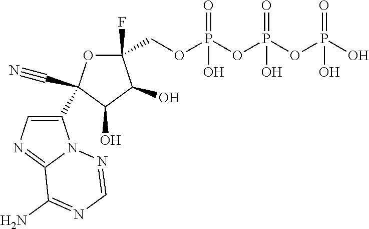 [SMILES: N#C[C@@]1(c2cnc3c(N)ncnn23)O[C@](F)(COP(=O)(O)OP(=O)(O)OP(=O)(O)O)[C@@H](O)[C@H]1O] | ((2S,3S,4R,5R)-5-(4- aminoimidazo[2,1-f] [1,2,4]triazin- 7-yl)-5-cyano-2-fluoro-3,4- dihydroxytetrahydrofuran-2- yl)methyl tetrahydrogen triphosphate |
| 82 | 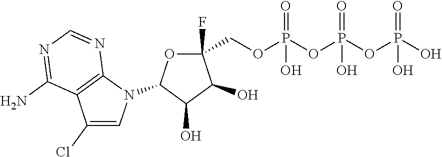 [SMILES: Nc1ncnc2c1c(Cl)cn2[C@@H]1O[C@](F)(COP(=O)(O)OP(=O)(O)OP(=O)(O)O)[C@@H](O)[C@H]1O] | ((2S,3S,4R,5R)-5-(4-amino-5- chloro-7H-pyrrolo[2,3- d]pyrimidin-7-yl)-2-fluoro-3,4- dihydroxytetrahydrofuran-2- yl)methyl tetrahydrogen triphosphate |
| 83 | 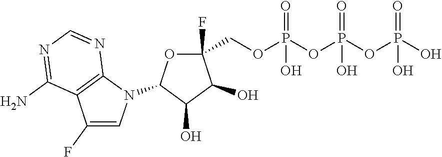 [SMILES: Nc1ncnc2c1c(F)cn2[C@@H]1O[C@](F)(COP(=O)(O)OP(=O)(O)OP(=O)(O)O)[C@@H](O)[C@H]1O] | ((2S,3S,4R,5R)-5-(4-amino-5- fluoro-7H-pyrrolo[2,3- d]pyrimidin-7-yl)-2-fluoro-3,4- dihydroxytetrahydrofuran-2- yl)methyl tetrahydrogen triphosphate |
| 84 | 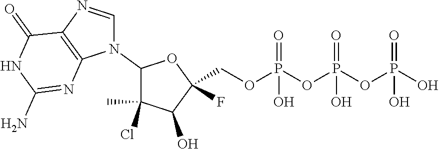 [SMILES: C[C@]1(Cl)C(n2cnc3c(=O)[nH]c(N)nc32)O[C@](F)(COP(=O)(O)OP(=O)(O)OP(=O)(O)O)[C@H]1O] | ((2S,3R,4R,5R)-5-(2-amino-6- oxo-1,6-dihydro-9H-purin-9-yl)-4- chloro-2-fluoro-3-hydroxy-4- methyltetrahydrofuran-2-yl)methyl tetrahydrogen triphosphate |
| 85 | 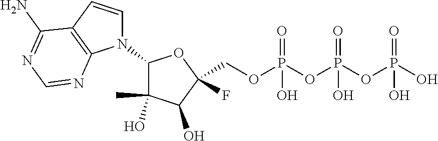 [SMILES: C[C@@]1(O)[C@H](n2ccc3c(N)ncnc32)O[C@](F)(COP(=O)(O)OP(=O)(O)OP(=O)(O)O)[C@H]1O] | ((2S,3S,4S,5R)-5-(4-amino- 7H-pyrrolo[2,3-d]pyrimidin-7-yl)- 2-fluoro-3,4-dihydroxy-4- methyltetrahydrofuran-2-yl)methyl tetrahydrogen triphosphate |

### EXAMPLES

### General Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N‘N’-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H4SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford).

### Example 1

### Synthesis of Compound 1

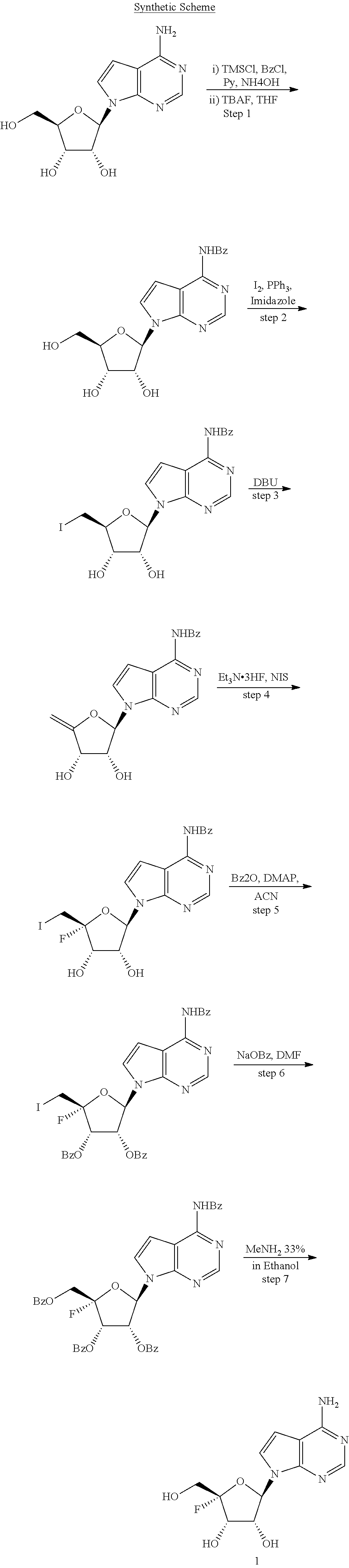

Step 1: Synthesis of N-(7-((2R,3R,4S,5R)-3,4-dihydroxy-5-(hydroxymethyl)tetrahydrofuran-2-yl)-7H-pyrrolo[2,3-d]pyrimidin-4-yl)benzamide: To a stirred solution of (2R,3R,4S,5R)-2-(4-amino-7H-pyrrolo[2,3-d]pyrimidin-7-yl)-5-(hydroxymethyl)tetrahydrofuran-3,4-diol (1.2 g, 4.51 mmol) in anhydrous pyridine (10 mL) was added TMSCl (2.57 mL, 20.3 mmol) dropwise at 0° C. and the resulting reaction mixture was stirred for 30 min at the same temperature. Then benzoyl chloride (848 μL, 6.76 mmol) was added dropwise. The reaction mixture was stirred at room temperature for 16 h. After completion of the reaction, it was quenched with water (0.7 mL), followed by 25% aqueous NH₄OH (1.8 ml) and the resulting mixture was further stirred for 10 min. Solvent was removed in vacuo and the residue was diluted with water (20 mL) and extracted with EtOAc (3×20 mL). The combined organic layer was dried over anhydrous sodium sulfate and filtrate was concentrated in vacuo to get a crude product (1.7 g).

To a solution of the crude product in anhydrous THE (15 mL) was added TBAF (2.5 mL, 9.12 mmol) at 0° C. The resulting reaction mixture was stirred for 1 h at room temperature. After completion of the reaction, solvent was removed in vacuo. The residue was purified by Silica gel column chromatography using 5-8% MeOH in DCM as eluent to get N-(7-((2R,3R,4S,5R)-3,4-dihydroxy-5-(hydroxymethyl)tetrahydrofuran-2-yl)-7H-pyrrolo[2,3d]pyrimidin-4-yl)benzamide (700 mg, 50%) as a white solid. LCMS(ESI): m/z 371.05 [M+H]⁺.

Step 2: Synthesis of N-(7-((2R,3R,4S,5S)-3,4-dihydroxy-5-(iodomethyl)tetrahydrofuran-2-yl)-7H-pyrrolo[2,3-d]pyrimidin-4-yl)benzamide: To a stirred solution of N-(7-((2R,3R,4S,5R)-3,4-dihydroxy-5-(hydroxymethyl)tetrahydrofuran-2-yl)-7H-pyrrolo[2,3-d]pyrimidin-4-yl)benzamide (0.6 g, 1.62 mmol), PPh₃ (1.19 g, 4.54 mmol) and 1H-imidazole (309 mg, 4.54 mmol) in anhydrous THE (8 mL) was added a solution of I₂ (905 mg, 3.56 mmol) in THE (2 ml) at 0° C. The reaction mixture was stirred at room temperature for 16 h. After completion of the reaction, mixture was quenched with saturated aqueous sodium thiosulphate solution (10 mL) and extracted with EtOAc (3×20 mL). The combined organic layer was dried over anhydrous sodium sulfate and concentrated in vacuo. The residue was purified by Silica gel column chromatography using 5% MeOH in DCM eluent to afford N-(7-((2R,3R,4S,5S)-3,4-dihydroxy-5-(iodomethyl)tetrahydrofuran-2-yl)-7H-pyrrolo[2,3-d]pyrimidin-4-yl)benzamide (0.6 g, 77%) as an off-white solid. LCMS(ESI): m/z 481.00 [M+H]⁺.

Step 3: Synthesis of N-(7-((2R,3R,4S)-3,4-dihydroxy-5-methylenetetrahydrofuran-2-yl)-7H-pyrrolo[2,3-d]pyrimidin-4-yl)benzamide: To a stirred solution of N-(7-((2R,3R,4S,5S)-3,4-dihydroxy-5-(iodomethyl)tetrahydrofuran-2-yl)-7H-pyrrolo[2,3-d]pyrimidin-4-yl)benzamide (590 mg, 1.23 mmol) in anhydrous THE (8 mL) was added 1,8-diazabicyclo[5.4.0]undec-7-ene (275 μL, 1.84 mmol) at room temperature. The reaction mixture was stirred at 50° C. for 2 h. After completion of the reaction, solvent was removed in vacuo and the residue was purified by Silica gel column chromatography using 3-5% MeOH in DCM eluent to get N-(7-((2R,3R,4S)-3,4-dihydroxy-5-methylenetetrahydrofuran-2-yl)-7H-pyrrolo[2,3-d]pyrimidin-4-yl)benzamide (250 mg, 58%) as an off-white solid. LCMS(ESI): m/z 353.00 [M+H]⁺.

Step 4: Synthesis of N-(7-((2R,3R,4S,5R)-5-fluoro-3,4-dihydroxy-5-(iodomethyl)tetrahydrofuran-2-yl)-7H-pyrrolo[2,3-d]pyrimidin-4-yl)benzamide: To a stirred solution of (7-((2R,3R,4S)-3,4-dihydroxy-5-methylenetetrahydrofuran-2-yl)-7H-pyrrolo[2,3-d]pyrimidin-4-yl)benzamide (250 mg, 0.710 mmol) in anhydrous ACN (12 mL) was added TEA 3HF (139 μL, 0.851 mmol) at 0° C. Then, a solution of NIS (192 mg, 0.851 mmol) in ACN (3 mL) was added and the resulting reaction mixture was stirred for 40 minutes at 0° C. The reaction was warmed up to room temperature and stirred for an additional 40 minutes to form a solid precipitate. After completion of the reaction, the precipitate was filtered and washed with ACN to afford N-(7-((2R,3R,4S,5R)-5-fluoro-3,4-dihydroxy-5-(iodomethyl)tetrahydrofuran-2-yl)-7H-pyrrolo[2,3-d]pyrimidin-4-yl) benzamide (200 mg, 57%) as a white solid. LCMS(ESI): m/z 498.75 [M+H]⁺.

Step 5: Synthesis of (2R,3S,4R,5R)-5-(4-benzamido-7H-pyrrolo[2,3-d]pyrimidin-7-yl)-2-fluoro-2-(iodomethyl)tetrahydrofuran-3,4-diyl dibenzoate: To a stirred solution of N-(7-((2R,3R,4S,5R)-5-fluoro-3,4-dihydroxy-5-(iodomethyl)tetrahydrofuran-2-yl)-7H-pyrrolo[2,3-d]pyrimidin-4-yl)benzamide (190 mg, 0.381 mmol) in anhydrous ACN (5 mL) was added benzoic anhydride (216 mg, 0.953 mmol) and DMAP (9.39 mg, 0.076 mmol) at 0° C. The reaction mixture was stirred at room temperature for 6 h. After completion of the reaction, the mixture was diluted with EtOAc (15 mL) and washed with water (15 mL). The combined organic layer was dried over anhydrous sodium sulfate and concentrated in vacuo. The residue was purified by Silica gel column chromatography using 10-40% EtOAc in heptane as eluent to afford (2R,3S,4R,5R)-5-(4-benzamido-7H-pyrrolo[2,3-d]pyrimidin-7-yl)-2-fluoro-2-(iodomethyl) tetrahydrofuran-3,4-diyl dibenzoate (180 mg, 67%) as an off-white solid. LCMS(ESI): m/z 706.80 [M+H]⁺.

Step 6: Synthesis of (2S,3S,4R,5R)-5-(4-benzamido-7H-pyrrolo[2,3-d]pyrimidin-7-yl)-2-((benzoyloxy)methyl)-2-fluorotetrahydrofuran-3,4-diyl dibenzoate: To a stirred solution of (2R,3 S,4R,5R)-5-(4-benzamido-7H-pyrrolo[2,3-d]pyrimidin-7-yl)-2-fluoro-2-(iodomethyl) tetrahydrofuran-3,4-diyl dibenzoate (170 mg, 0.241 mmol) in DMF (17 mL) was added sodium benzoate (277 mg, 1.93 mmol) at room temperature. The reaction was stirred at 120° C. for 16 h. After completion of the reaction, the mixture was diluted with EtOAc (20 mL) and washed with ice-cold water (3×10 mL). The combined organic layer was dried over anhydrous sodium sulphate and concentrated in vacuo. The residue was purified by Silica gel column chromatography using 20-50% EtOAc in heptane as eluent to afford (2S,3S,4R,5R)-5-(4-benzamido-7H-pyrrolo[2,3-d]pyrimidin-7-yl)-2-(benzoyloxy)methyl)-2-fluorotetrahydrofuran-3,4-diyl dibenzoate (130 mg, 77%) as a white solid. LCMS(ESI): m/z 700.95 [M+H]⁺.

Step 7: Synthesis of (2S,3S,4R,5R)-5-(4-amino-7H-pyrrolo[2,3-d]pyrimidin-7-yl)-2-fluoro-2-(hydroxymethyl)tetrahydrofuran-3,4-diol (1): A solution of (2S,3S,4R,5R)-5-(4-benzamido-7H-pyrrolo[2,3-d]pyrimidin-7-yl)-2-((benzoyloxy)methyl)-2-fluorotetrahydrofuran-3,4-diyl dibenzoate (40 mg, 0.057 mmol) in methyl amine (33% in Ethanol, 2 mL) was stirred at room temperature for 16 h. After completion of the reaction, the reaction mixture was concentrated in vacuo at low temperature. The residue was purified using trituration in 5% MeOH in DCM to afford (2S,3S,4R,5R)-5-(4-amino-7H-pyrrolo[2,3-d]pyrimidin-7-yl)-2-fluoro-2-(hydroxymethyl)tetrahydrofuran-3,4-diol (4.5 mg, 28%) as an off-white solid. ¹H-NMR (400 MHz, DMSO-d₆): δ 8.07 (s, 1H), 7.26 (d, J=3.6, 1H), 7.08 (s, 2H), 6.61 (d, J=3.6 Hz, 1H), 6.33 (d, J=3.2 Hz, 1H), 5.66 (d, J=5.6 Hz, 1H), 5.38 (t, J=6.4 Hz, 1H), 5.16 (d, J=8.8 Hz, 1H), 4.52-4.44 (m, 1H), 4.38-4.34 (m, 1H), 3.54 (t, J=6.4 Hz, 2H). LCMS(ESI): m/z 285.15 [M+H]⁺.

Procedure analogous to those for the synthesis of compound 1 were used for the synthesis of compound 6, 10, 11, 12, 14-18, 21-26, 28, 31, 33, 35-45, 50, 52-55 and 72 et al.

### Example 2

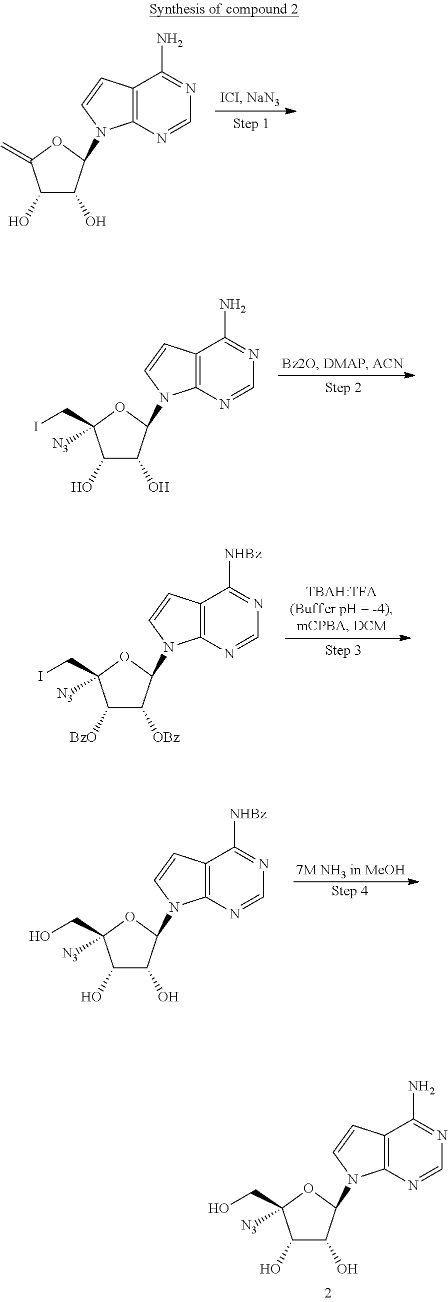

Step 1: Synthesis of (2S,3S,4R,5R)-5-(4-amino-7H-pyrrolo[2,3-d]pyrimidin-7-yl)-2-azido-2-(iodomethyl)tetrahydrofuran-3,4-diol: To a stirred solution of iodine monochloride (654 mg, 4.03 mmol) in anhydrous DMF (4 mL) was added sodium azide (576 mg, 8.86 mmol) at 0° C. The resulting mixture was stirred at room temperature for 30 minutes. The reaction mixture was again cooled to 0° C. and a solution of (2R,3R,4S)-2-(4-amino-7H-pyrrolo[2,3-d]pyrimidin-7-yl)-5-methylenetetrahydrofuran-3,4-diol (0.4 g, 1.61 mmol) in anhydrous DMF (2 mL) was added dropwise. The reaction was further stirred at room temperature for 2 h. After completion of the reaction, the reaction was quenched with saturated sodium thiosulphate aqueous solution (10 mL) and extracted with EtOAc (3×15 mL). The combined organic layer was dried over anhydrous sodium sulphate and concentrated in vacuo. The residue was purified by Silica gel column chromatography using 0-10% MeOH in DCM as eluent to afford (2S,3S,4R,5R)-5-(4-amino-7H-pyrrolo[2,3-d]pyrimidin-7-yl)-2-azido-2-(iodomethyl)tetrahydrofuran-3,4-diol (105 mg, 16%) as a Yellow sticky gum. LCMS(ESI): m/z 417.85 [M+H]⁺.

Step 2: Synthesis of (2S,3S,4R,5R)-2-azido-5-(4-benzamido-7H-pyrrolo[2,3-d]pyrimidin-7-yl)-2-(iodomethyl)tetrahydrofuran-3,4-diyl dibenzoate: To a stirred solution of (2S,3S,4R,5R)-5-(4-amino-7H-pyrrolo[2,3-d]pyrimidin-7-yl)-2-azido-2-(iodomethyl)tetrahydrofuran-3,4-diol (130 mg, 0.312 mmol) and DMAP (11.4 mg, 0.093 mmol) in anhydrous pyridine (2.6 mL) was added benzoyl chloride (163 μL, 1.4 mmol) at 0° C. The resulting mixture was stirred at room temperature for 1 h. After completion of the reaction, the reaction was diluted with 10% sodium bicarbonate aqueous solution (10 mL) and extracted with EtOAc (3×20 mL). The combined organic layer was dried over anhydrous sodium sulphate and concentrated in vacuo. The residue was purified by Silica gel column chromatography using 30-50% EtOAc in heptane as eluent to afford (2S,3S,4R,5R)-2-azido-5-(4-benzamido-7H-pyrrolo[2,3-d]pyrimidin-7-yl)-2-(iodomethyl)tetrahydrofuran-3,4-diyl dibenzoate (110 mg, 48%) as a white solid. LCMS(ESI): m/z 728.09 [M−H]⁻.

Step 3: Synthesis of (2R,3S,4R,5R)-2-azido-5-(4-benzamido-7H-pyrrolo[2,3-d]pyrimidin-7-yl)-2-(hydroxymethyl)tetrahydrofuran-3,4-diyl dibenzoate: Bu₄NOH (55%) (6 mL) was adjusted to pH=4 by adding TFA (approx. 1.2 mL). The resulting buffer solution (2 mL) was added to a stirred solution of (2S,3S,4R,5R)-2-azido-5-(4-benzamido-7H-pyrrolo[2,3-d]pyrimidin-7-yl)-2-(iodomethyl)tetrahydrofuran-3,4-diyl dibenzoate (105 mg, 0.144 mmol) in anhydrous DCM (2 mL) at 0° C. mCPBA (149 mg, 0.864 mmol) was added in portions-wise under vigorous stirring. The reaction was stirred at room temperature for 16 h. After completion of the reaction, the mixture was diluted with DCM (10 mL) and washed with saturated aqueous sodium bicarbonate solution. The organic layer was dried over anhydrous sodium sulfate and concentrated in vacuo. The residue was purified by Silica gel column chromatography using 10-60% EtOAc in heptane as eluent to afford (2R,3S,4R,5R)-2-azido-5-(4-benzamido-7H-pyrrolo[2,3-d]pyrimidin-7-yl)-2-(hydroxymethyl)tetrahydrofuran-3,4-diyl dibenzoate (50 mg, 56%) as a white solid. LCMS(ESI): m/z 620.1 [M+H]⁺.

Step 4: Synthesis of (2R,3S,4R,5R)-5-(4-amino-7H-pyrrolo[2,3-d]pyrimidin-7-yl)-2-azido-2-(hydroxymethyl)tetrahydrofuran-3,4-diol (2): A solution of (2R,3R,4S,5R)-5-azido-2-{4-benzamido-7H-pyrrolo[2,3-d]pyrimidin-7-yl}-4-(benzoyloxy)-5-(hydroxymethyl)oxolan-3-yl benzoate (50 mg, 0.080 mmol) in NH₃ (7M in MeOH, 1 mL) was stirred at room temperature for 16 h. After completion of the reaction, the mixture was concentrated in vacuo. The residue was purified by Reverse Phase prep-HPLC to get the desired (2R,3S,4R,5R)-5-{4-amino-7H-pyrrolo[2,3-d]pyrimidin-7-yl}-2-azido-2-(hydroxymethyl)oxolane-3,4-diol (4.5 mg, 18%) as a white solid. ¹H-NMR (400 MHz, DMSO-d₆ with D₂O@HT): δ 8.06 (s, 1H), 7.29 (d, J=3.6, 1H), 6.62 (d, J=3.2 Hz, 1H), 6.25 (d, J=6 Hz, 1H), 4.61 (t, J=6.4 Hz, 1H), 4.33 (d, J=5.6 Hz, 1H), 3.56 (d, J=12 Hz, 1H), 3.46 (d, J=11.6 Hz, 1H). LCMS(ESI): m/z 308.2 [M+H]⁺.

Procedure analogous to those for the synthesis of compound 2 were used for the synthesis of compound 7, 8 and 9 et al.

### Example 3

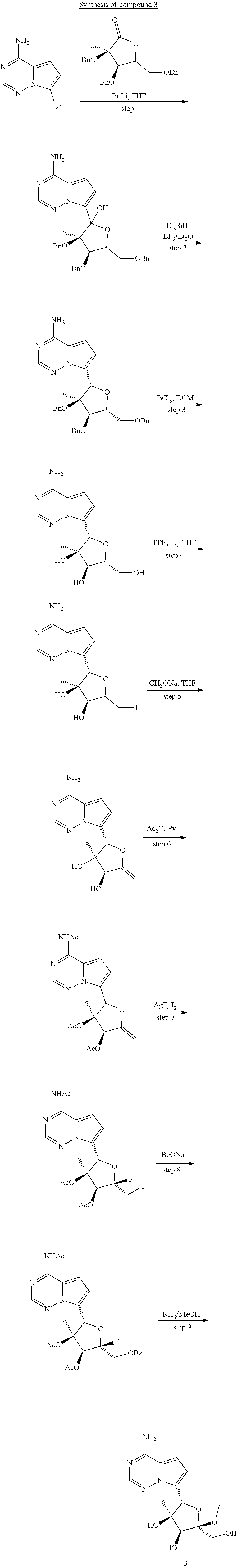

Step 1: Synthesis of (3R,4R,5R)-2-(4-aminopyrrolo[2,1-f][1,2,4]triazin-7-yl)-3,4-bis(benzyloxy)-5-((benzyloxy)methyl)-3-methyltetrahydrofuran-2-ol: To a stirred solution of 7-bromopyrrolo[2,1-f][1,2,4]triazin-4-amine (16.25 g, 76.298 mmol) in THE (380 mL) was added chloro[2-(chlorodimethylsilyl)ethyl]dimethylsilane (16.42 g, 76.298 mmol) and the resulting reaction mixture was stirred for 10 min at 0° C. under nitrogen atmosphere followed by the addition of 2,2,6,6-tetramethylpiperidine (10.78 g, 76.298 mmol) dropwise at the same temperature. The reaction was stirred for 30 min at 0° C. and then cooled to −78° C. A solution of n-BuLi (240 mL, 1.6 M in hexanes) was added dropwise over 30 min. The reaction mixture was stirred for 1 h at −78° C. under nitrogen atmosphere. To the above mixture was added a solution of (3R,4R,5R)-3,4-bis(benzyloxy)-5-[(benzyloxy)methyl]-3-methyloxolan-2-one (30 g, 69.362 mmol) in THF (100 mL) dropwise over 5 min at −78° C. The resulting reaction mixture was stirred for additional 2 h at −78° C. The reaction was quenched with saturated NH₄Cl aqueous solution at −78° C. The resulting mixture was extracted with EtOAc (3×500 mL). The combined organic layer was washed with brine (1×30 mL) and dried over anhydrous sodium sulfate. After filtration, the filtrate was concentrated in vacuo. The residue was purified by silica gel column chromatography, eluted with PE/THF (2:1) to afford (3R,4R,5R)-2-(4-aminopyrrolo[2,1-f][1,2,4]triazin-7-yl)-3,4-bis(benzyloxy)-5-((benzyloxy)methyl)-3-methyltetrahydrofuran-2-ol as a yellow oil (24.0 g, 61%). LCMS(ESI): m/z 589.3 [M+H]⁺.

Step 2: Synthesis of 7-((2S,3S,4R,5R)-3,4-bis(benzyloxy)-5-((benzyloxy)methyl)-3-methyltetrahydrofuran-2-yl)pyrrolo[2,1-f][1,2,4]triazin-4-amine: To a stirred solution of (3R,4R,5R)-2-{4-aminopyrrolo[2,1-f][1,2,4]triazin-7-yl}-3,4-bis(benzyloxy)-5-[(benzyloxy) methyl]-3-methyloxolan-2-ol (22 g, 38.824 mmol) in DCM (200 mL) was added Et₃SiH (18.06 g, 155.296 mmol) and the resulting mixture was stirred for 5 min at 0° C. under nitrogen atmosphere followed by the addition of BF₃·Et₂O (5.27 g, 77.648 mmol) dropwise at 0° C. The reaction was stirred for 1 h at room temperature under nitrogen atmosphere. After completion of the reaction, the reaction was quenched with saturated NH₄Cl aqueous solution at 0° C. The resulting mixture was extracted with EtOAc (3×500 mL). The combined organic layer was washed with brine (1×30 mL) and dried over anhydrous sodium sulfate. The residue was purified by silica gel column chromatography, eluted with PE/THF (2:1) to afford 7-((2S,3S,4R,5R)-3,4-bis(benzyloxy)-5-((benzyloxy)methyl)-3-methyltetrahydrofuran-2-yl)pyrrolo[2,1-f][1,2,4]triazin-4-amine as a yellow oil (21.0 g, 98%). LCMS(ESI): m/z 551.3 [M+H]⁺.

Step 3: Synthesis of (2S,3R,4R,5R)-2-(4-aminopyrrolo[2,1-f][1,2,4]triazin-7-yl)-5-(hydroxymethyl)-3-methyltetrahydrofuran-3,4-diol: To a stirred solution of 7-[(2S,3S,4R,5R)-3,4-bis(benzyloxy)-5-[(benzyloxy)methyl]-3-methyloxolan-2-yl]pyrrolo[2,1-f][1,2,4]triazin-4-amine (8 g, 14.528 mmol) in DCM (100 mL) was added boron trichloride (17.02 g, 145.3 mmol) and the resulting reaction mixture was stirred for 3 min at 0° C. under nitrogen atmosphere. The reaction was stirred for 30 min at room temperature under nitrogen atmosphere. After completion of the reaction, the reaction was quenched with saturated sodium bicarbonate aqueous solution (50 mL) at 0° C. The resulting mixture was separated with separating funnel. The water phase was purified with reversed-phase flash chromatography. The fractions was lyophilized and then further purified by Prep-Chiral SFC to give (2S,3R,4R,5R)-2-(4-aminopyrrolo[2,1-f][1,2,4]triazin-7-yl)-5-(hydroxymethyl)-3-methyltetrahydrofuran-3,4-diol (1.56 g, 38%) (the major peak in SFC). ¹H NMR (400 MHz, DMSO-d₆) δ 7.82 (s, 1H), 7.64 (s, 2H), 6.84 (d, J=4.4 Hz, 1H), 6.70 (d, J=4.4 Hz, 1H), 5.39 (s, 1H), 3.78-3.73 (m, 2H), 3.69 (d, J=8.0 Hz, 1H), 3.61-3.57 (m, 1H), 0.79 (s, 3H). LCMS(ESI): m/z 281.2 [M+H]⁺.

Step 4: Synthesis of (2S,3R,4R,5S)-2-(4-aminopyrrolo[2,1-f][1,2,4]triazin-7-yl)-5-(iodomethyl)-3-methyltetrahydrofuran-3,4-diol: A solution of (2S,3R,4R,5R)-2-{4-aminopyrrolo[2,1-f][1,2,4]triazin-7-yl}-5-(hydroxymethyl)-3-methyloxolane-3,4-diol (1.51 g, 5.387 mmol) in THE (80 mL) was treated with triphenylphosphine (3.53 g, 13.458 mmol), imidazole (526 mg, 7.726 mmol) and pyridine (3.0 mL, 37.28 mmol) at room temperature. To the above mixture was added a solution of iodine (2.73 g, 10.774 mmol) in THF (5 mL) dropwise over 15 min at 0° C. and the resulting mixture was stirred for 4 h at room temperature. The resulting mixture was concentrated in vacuo. The residue was purified by silica gel column chromatography, eluted with DCM/MeOH (10:1) to afford (2S,3R,4R,5S)-2-{4-aminopyrrolo[2,1-f][1,2,4]triazin-7-yl}-5-(iodomethyl)-3-methyloxolane-3,4-diol (1.77 g, 84%) as a brown solid. LCMS(ESI): m/z 391.0 [M+H]⁺.

Step 5: Synthesis of (2S,3R,4S)-2-(4-aminopyrrolo[2,1-f][1,2,4]triazin-7-yl)-3-methyl-5-methylenetetrahydrofuran-3,4-diol: To a stirred solution of (2S,3R,4R,5S)-2-{4-aminopyrrolo[2,1-f][1,2,4]triazin-7-yl}-5-(iodomethyl)-3-methyloxolane-3,4-diol (1.77 g, 4.537 mmol) in THE (20 mL) was added sodium methoxide (1.77 mL, 9.558 mmol) dropwise at 0° C. and the resulting reaction mixture was stirred for 2 h at 60° C. The reaction mixture was concentrated under reduced pressure and was extracted with EtOAc (3×50 mL). The combined organic layer was washed with brine (1×10 mL) and dried over anhydrous sodium sulfate. The solvent was remove in vacuo and the residue was purified by silica gel column chromatography, eluted with DCM/MeOH (10:1) to afford (2S,3R,4S)-2-(4-aminopyrrolo[2,1-f][1,2,4]triazin-7-yl)-3-methyl-5-methylenetetrahydrofuran-3,4-diol (480.0 mg, 40%) as a yellow oil. LCMS(ESI): m/z 263.3 [M+H]⁺.

Step 6: Synthesis of (2S,3S,4R)-2-(4-acetamidopyrrolo[2,1-f][1,2,4]triazin-7-yl)-3-methyl-5-methylenetetrahydrofuran-3,4-diyl diacetate: To a stirred solution of (2S,3R,4S)-2-{4-aminopyrrolo[2,1-f][1,2,4]triazin-7-yl}-3-methyl-5-methylideneoxolane-3,4-diol (400 mg, 1.525 mmol) in anhydrous pyridine (5 mL) was added Ac₂O (934.21 mg, 9.150 mmol) at 0° C. The reaction was stirred at room temperature for 6 h. After completion of the reaction, the reaction mixture was diluted with EtOAc (15 mL) and washed with water (15 mL). The organic layer was separated and dried over anhydrous sodium sulfate and concentrated in vacuo. The residue was purified by silica column chromatography using 10-40% EtOAc in heptane as eluent to afford (3R,4S,5S)-4-(acetyloxy)-5-{4-acetamidopyrrolo[2,1-f][1,2,4]triazin-7-yl}-4-methyl-2-methylideneoxolan-3-yl acetate (270 mg, 46%) as an off-white solid. LCMS(ESI): m/z 389.2 [M+H]⁺.

Step 7: Synthesis of (2R,3S,4S,5S)-5-(4-acetamidopyrrolo[2,1-f][1,2,4]triazin-7-yl)-2-fluoro-2-(iodomethyl)-4-methyltetrahydrofuran-3,4-diyl diacetate: To a stirred solution of (3R,4S,5S)-4-(acetyloxy)-5-{4-acetamidopyrrolo[2,1-f][1,2,4]triazin-7-yl}-4-methyl-2-methylideneoxolan-3-yl acetate (270 mg, 0.695 mmol) in anhydrous DCM (3 mL, 47.192 mmol) was added AgF (440.99 mg, 3.475 mmol) at 0° C. followed by a solution of I₂ (352.89 mg, 1.390 mmol) in DCM. The reaction mixture was stirred for 10 minutes at 0° C., then warmed up to room temperature and stirred for another 2 h to form a solid precipitate. After completion of the reaction, the solid precipitated was filtered and washed with ACN to afford (2R,3S,4S,5S)-4-(acetyloxy)-5-{4-acetamidopyrrolo[2,1-f][1,2,4]triazin-7-yl}-2-fluoro-2-(iodomethyl)-4-methyloxolan-3-yl acetate (245 mg, 66%) as a white solid. LCMS(ESI): m/z 535.1 [M+H]⁺.

Step 8: Synthesis of (2S,3S,4S,5S)-5-(4-acetamidopyrrolo[2,1-f][1,2,4]triazin-7-yl)-2-((benzoyloxy)methyl)-2-fluoro-4-methyltetrahydrofuran-3,4-diyl diacetate: To a stirred solution of (2R,3S,4S,5S)-4-(acetyloxy)-5-{4-acetamidopyrrolo[2,1-f][1,2,4]triazin-7-yl}-2-fluoro-2-(iodomethyl)-4-methyloxolan-3-yl acetate (200 mg, 0.374 mmol) in DMSO (5 mL) was added sodium benzoate (539.43 mg, 3.740 mmol) and 15-crown-5 ether (1.65 g, 7.480 mmol) at room temperature. The resulting reaction mixture was stirred at 80° C. for 48 h. After completion of the reaction, the mixture was diluted with EtOAc (20 mL) and washed with ice-cold water (3×10 mL). The organic layer was separated, dried over anhydrous sodium sulphate and concentrated in vacuo. The residue was purified by silica column chromatography using 20-50% EtOAc in heptane as eluent to afford [(2S,3S,4S,5S)-3,4-bis(acetyloxy)-5-{4-acetamidopyrrolo[2,1-f][1,2,4]triazin-7-yl}-2-fluoro-4-methyloxolan-2-yl]methyl benzoate (50 mg, 25%) as a white solid. LCMS(ESI): m/z 529.1 [M+H]⁺.

Step 9: Synthesis of (2R,3S,4R,5S)-5-(4-aminopyrrolo[2,1-f][1,2,4]triazin-7-yl)-2-(hydroxymethyl)-2-methoxy-4-methyltetrahydrofuran-3,4-diol (3): A solution of [(2S,3S,4S,5S)-3,4-bis(acetyloxy)-5-{4-acetamidopyrrolo[2,1-f][1,2,4]triazin-7-yl}-2-fluoro-4-methyloxolan-2-yl]methyl benzoate (40 mg, 0.076 mmol) in NH₃ (7M in MeOH, 5 mL) was stirred for 6 h at room temperature. After completion of the reaction, the reaction mixture was concentrated in Vacuo. The residue was purified by Prep-HPLC to afford (2R,3S,4R,5S)-5-{4-aminopyrrolo[2,1-f][1,2,4]triazin-7-yl}-2-(hydroxymethyl)-2-methoxy-4-methyloxolane-3,4-diol (1.4 mg, 6%) as a white solid. ¹H NMR (400 MHz, DMSO-d₆) δ 7.82 (s, 1H), 7.64 (s, 2H), 6.84 (d, J=4.4 Hz, 1H), 6.72 (d, J=4.4 Hz, 1H), 5.55 (s, 1H), 4.95 (d, J=6.0 Hz, 1H), 4.49 (d, J=9.2 Hz, 1H), 4.32 (s, 1H), 3.93 (d, J=9.2 Hz, 1H), 3.69 (d, J=5.6 Hz, 1H), 3.48 (d, J=11.2 Hz, 1H), 3.27 (s, 3H), 0.75 (s, 3H). LCMS(ESI): m/z 311.1 [M+H]⁺. Procedure analogous to those for the synthesis of compound 3 were used for the synthesis of compound 19, 34 and 51 et al.

### Example 4

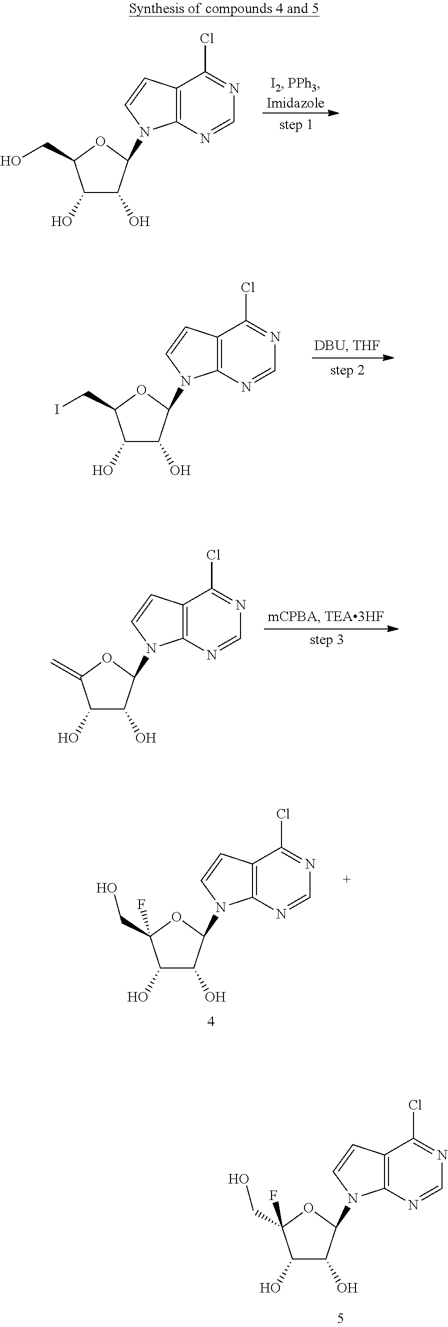 [SMILES: OC[C@H]1O[C@@H](n2ccc3c(Cl)ncnc32)[C@H](O)[C@@H]1O ; O[C@@H]1[C@H](O)[C@@H](CI)O[C@H]1n1ccc2c(Cl)ncnc21 ; C=C1O[C@@H](n2ccc3c(Cl)ncnc32)[C@H](O)[C@@H]1O ; OC[C@@]1(F)O[C@@H](n2ccc3c(Cl)ncnc32)[C@H](O)[C@@H]1O ; OC[C@]1(F)O[C@@H](n2ccc3c(Cl)ncnc32)[C@H](O)[C@@H]1O]

Step 1: Synthesis of (2R,3R,4S,5S)-2-(4-chloro-7H-pyrrolo[2,3-d]pyrimidin-7-yl)-5-(iodomethyl)tetrahydrofuran-3,4-diol: To a solution of (2R,3R,4S,5R)-2-(4-chloro-7H-pyrrolo[2,3-d]pyrimidin-7-yl)-5-(hydroxymethyl)tetrahydrofuran-3,4-diol (1.06 g, 3.7 mmol) in anhydrous THE (12 mL) was added PPh₃ (1.2 g, 4.5 mmol) and imidazole (503 mg, 7.4 mmol) and the resulting reaction mixture was stirred for 30 min at room temperature. Iodine (1.15 g, 4.5 mmol) was added to the above reaction mixture at 0° C. The reaction was stirred at room temperature for 12 h. After completion of the reaction, the reaction was quenched with saturated NaHCO₃ aqueous solution. The resulting mixture was concentrated in Vacuo. The residue was diluted with DCM and extracted three times. The combined organic layer was dried over anhydrous sodium sulfate and filtrate was concentrated in vacuo to get a crude product-(2R,3R,4S,5S)-2-(4-chloro-7H-pyrrolo[2,3-d]pyrimidin-7-yl)-5-(iodomethyl)tetrahydrofuran-3,4-diol. LCMS(ESI): m/z 396.1 [M+H]⁺.

Step 2: Synthesis of (2R,3R,4S)-2-(4-chloro-7H-pyrrolo[2,3-d]pyrimidin-7-yl)-5-methylenetetrahydrofuran-3,4-diol: To a solution of (2R,3R,4S,5S)-2-(4-chloro-7H-pyrrolo[2,3-d]pyrimidin-7-yl)-5-(iodomethyl)tetrahydrofuran-3,4-diol (crude product from the step 1) in anhydrous THE (15 mL) was added DBU (8.6 mL, 55.6 mmol) at 0° C. The resulting reaction mixture was stirred at room temperature for 12 h. After completion of the reaction, AcOH was added to adjust pH to 7 followed by addition of water. The resulting mixture was extracted with EtOAc. The combined organic layer was dried over anhydrous sodium sulfate and concentrated in Vacuo to afford a crude product-(2R,3R,4S)-2-(4-chloro-7H-pyrrolo[2,3-d]pyrimidin-7-yl)-5-methylenetetrahydrofuran-3,4-diol. The crude product was used for next step without further purification. LCMS(ESI): m/z 268.0 [M+H]⁺.

Step 3: Synthesis of (2S,3S,4R,5R)-5-(4-chloro-7H-pyrrolo[2,3-d]pyrimidin-7-yl)-2-fluoro-2-(hydroxymethyl)tetrahydrofuran-3,4-diol (4) and (2R,3S,4R,5R)-5-(4-chloro-7H-pyrrolo[2,3-d]pyrimidin-7-yl)-2-fluoro-2-(hydroxymethyl)tetrahydrofuran-3,4-diol (5): To a stirred solution of (2R,3R,4S)-2-(4-chloro-7H-pyrrolo[2,3-d]pyrimidin-7-yl)-5-methylenetetrahydrofuran-3,4-diol (crude product from step 2, 230 mg, 0.86 mmol) in anhydrous DCM (6 mL) was added mCPBA (297 mg, 1.7 mmol) at 0° C. follow by TEA·3HF (0.71 ml, 4.3 mmol). The reaction mixture was stirred for 40 minutes at 0° C. After completion of the reaction, the reaction mixture was concentrated in Vacuo. The residue was purified by reserve phase Prep-HPLC to afford the desired compound 4 and 5 (3 mg, 6%; 3 mg, 6%). Compound 4: ¹H NMR (400 MHz, DMSO-d₆) δ 8.69 (d, J=1.4 Hz, 1H), 7.87 (d, J=3.7 Hz, 1H), 6.79 (d, J=3.7 Hz, 1H), 6.47 (d, J=3.0 Hz, 1H), 4.54 (dd, J=16.4, 6.4 Hz, 1H), 4.44 (t, J=4.8 Hz, 1H), 3.60-3.53 (m, 2H). LCMS(ESI): m/z 304.2 [M+H]⁺. Compound 5: 1H NMR (400 MHz, DMSO)¹H NMR (400 MHz, DMSO-d₆) δ 8.64 (s, 1H), 7.84-7.71 (d, 1H), 6.80 (d, J=3.8 Hz, 1H), 6.44 (t, J=7.1 Hz, 1H), 4.84 (dt, J=7.3, 3.5 Hz, 1H), 4.16-4.05 (m, 1H), 3.73 (s, 1H), 3.52-3.40 (m, 1H). LCMS(ESI): m/z 304.2 [M+H]⁺.

Procedure analogous to those for the synthesis of compound 4 were used for the synthesis of compound 13, 27 and 29 et al.

### Example 5

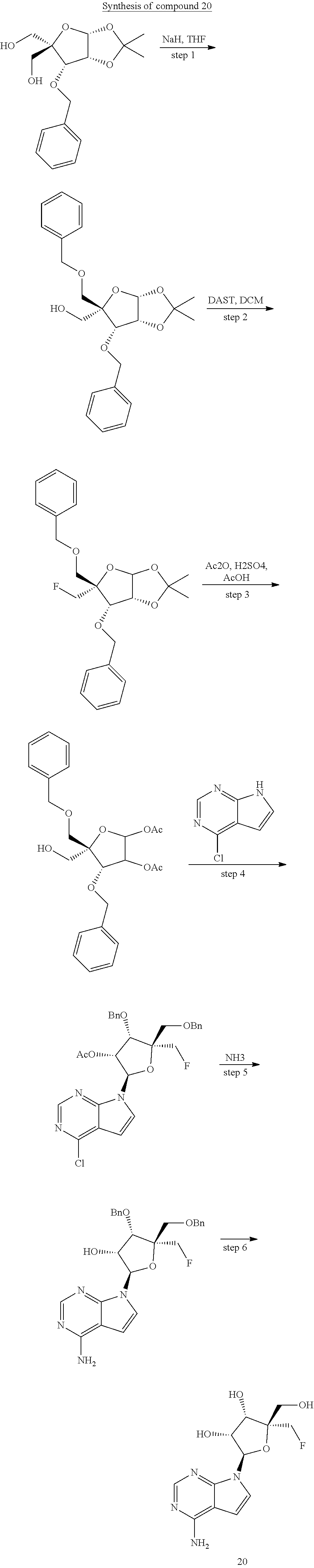

Step 1: Synthesis of ((5R,6S,6aR)-6-(benzyloxy)-5-((benzyloxy)methyl)-2,2-dimethyltetrahydrofuro[2,3-d][1,3]dioxol-5-yl)methanol: To a stirred solution of ((3aR,6S,6aR)-6-(benzyloxy)-2,2-dimethyltetrahydrofuro[2,3-d][1,3]dioxole-5,5-diyl)dimethanol (6 g, 19.35 mmol) and 60% NaH (1.08 g, 27.09 mmol) in anhydrous N,N-Dimethylformamide (60 mL) was added Benzyl bromide (3.62 g, 21.3 mmol) at 0° C. under N₂. The resulting mixture was stirred at rt for 1 h, then quenched with water and extracted with EtOAc. The combined organic layer was dried over anhydrous sodium sulfate and concentrated under reduced pressure. The residue was purified by silica gel column to afford ((5R,6S,6aR)-6-(benzyloxy)-5-((benzyloxy)methyl)-2,2-dimethyltetrahydrofuro[2,3-d][1,3]dioxol-5-yl)methanol (5.2 g, 67%) as a yellow solid. LCMS(ESI): m/z 423.0 [M+Na]⁺.

Step 2: Synthesis of (5R,6S,6aR)-6-(benzyloxy)-5-((benzyloxy)methyl)-5-(fluoromethyl)-2,2-dimethyltetrahydrofuro[2,3-d][1,3]dioxole: To a stirred solution of (3R,4R,5R)-2-(4-aminopyrrolo[2,1-f][1,2,4]triazin-7-yl)-3,4-bis(benzyloxy)-5-((benzyloxy)methyl)tetrahydrofuran-2-ol (5.2 g, 13 mmol) in Toluene (52 mL) was added DAST (6.28 g, 39 mmol) at 0° C. under N₂. The resulting mixture was stirred at 60° C. for 5 h, then quenched with water and extracted with EtOAc. The combined organic layer was dried over anhydrous sodium sulfate and concentrated under reduced pressure. The residue was purified by silica gel column to afford (5R,6S,6aR)-6-(benzyloxy)-5-((benzyloxy)methyl)-5-(fluoromethyl)-2,2-dimethyltetrahydrofuro[2,3-d][1,3]dioxole (2.7 g, 52%) as a yellow oil. LCMS(ESI): m/z 425.1 [M+Na]⁺.

Step 3: Synthesis of (3R,4S,5R)-4-(benzyloxy)-5-((benzyloxy)methyl)-5-(fluoromethyl)tetrahydrofuran-2,3-diyl diacetate: (5R,6S,6aR)-6-(benzyloxy)-5-((benzyloxy)methyl)-5-(fluoromethyl)-2,2-dimethyltetrahydrofuro[2,3-d][1,3]dioxole (2.7 g, 6.7 mmol) in Acetic acid (36 mL) was added Acetic anhydride (0.68 g, 6.7 mmol) and H₂SO₄ (65.7 mg, 0.67 mmol) at 10° C. The resulting mixture was stirred at rt for 1.5 h. After completion of the reaction, the reaction was quenched with H₂O and extracted with EtOAc. The combined organic layer was dried over anhydrous sodium sulfate and concentrated under reduced pressure to get crude product. The residue was purified by silica gel column to afford (3R,4S,5R)-4-(benzyloxy)-5-((benzyloxy)methyl)-5-(fluoromethyl)tetrahydrofuran-2,3-diyl diacetate (2.3 g, 77%) as a yellow solid. LCMS(ESI): m/z 469.0 [M+Na]⁺.

Step 4: Synthesis of (2R,3R,4S,5R)-4-(benzyloxy)-5-((benzyloxy)methyl)-2-(4-chloro-7H-pyrrolo[2,3-d]pyrimidin-7-yl)-5-(fluoromethyl)tetrahydrofuran-3-yl acetate: To a mixture of 4-chloro-7H-pyrrolo[2,3-d]pyrimidine (577.42 mg, 3.76 mmol) and BSA (764.9 mg, 3.76 mmol) in ACN (32 mL) was added (3R,4S,5R)-4-(benzyloxy)-5-((benzyloxy)methyl)-5-(fluoromethyl)tetrahydrofuran-2,3-diyl diacetate (1.6 g, 3.76 mmol) and TMSOTf (834.72 mg, 3.76 mmol). The reaction was stirred at 80° C. for 3 h. After completion of the reaction, the reaction was quenched with NaHCO₃(aq) and extracted with EtOAc. The combined organic layer was dried, concentrated and purified by silica gel column to give (2R,3R,4S,5R)-4-(benzyloxy)-5-((benzyloxy)methyl)-2-(4-chloro-7H-pyrrolo[2,3-d]pyrimidin-7-yl)-5-(fluoromethyl)tetrahydrofuran-3-yl acetate (215 mg, 11%) as a yellow solid. LCMS(ESI): m/z 540.0 [M+H]⁺.

Step 5: Synthesis of (2R,3R,4S,5R)-2-(4-amino-7H-pyrrolo[2,3-d]pyrimidin-7-yl)-4-(benzyloxy)-5-((benzyloxy)methyl)-5-(fluoromethyl)tetrahydrofuran-3-ol: To a solution of (2R,3R,4S,5R)-4-(benzyloxy)-5-((benzyloxy)methyl)-2-(4-chloro-7H-pyrrolo[2,3-d]pyrimidin-7-yl)-5-(fluoromethyl)tetrahydrofuran-3-yl acetate (215 mg, 0.4 mmol) in 1,4-dioxane (4 mL) was added NH₃H₂O (4 mL) and the resulting mixture was stirred at 100° C. for 25 h. The mixture was concentrated and purified by silica gel column to give (2R,3R,4S,5R)-2-(4-amino-7H-pyrrolo[2,3-d]pyrimidin-7-yl)-4-(benzyloxy)-5-((benzyloxy)methyl)-5-(fluoromethyl)tetrahydrofuran-3-ol (180 mg, 94% yield) as a yellow solid. LCMS (ESI): m/z 479.2 [M+H]⁺.

Step 6: Synthesis of (2R,3S,4R,5R)-5-(4-amino-7H-pyrrolo[2,3-d]pyrimidin-7-yl)-2-(fluoromethyl)-2-(hydroxymethyl)tetrahydrofuran-3,4-diol (20): To a stirred solution of (2R,3R,4S,5R)-2-(4-amino-7H-pyrrolo[2,3-d]pyrimidin-7-yl)-4-(benzyloxy)-5-((benzyloxy)methyl)-5-(fluoromethyl)tetrahydrofuran-3-ol (180 mg, 0.376 mmol) in DCM (3 mL) was added BCl₃ (3.8 mL, 3.76 mmol, 1M) at −78° C. under N₂. The resulting mixture was stirred at −78° C. for 2 h and then quenched with MeOH (10 mL). The excess solvent was removed under reduced pressure. The residue was purified by Prep-HPLC to get (2R,3S,4R,5R)-5-(4-amino-7H-pyrrolo[2,3-d]pyrimidin-7-yl)-2-(fluoromethyl)-2-(hydroxymethyl)tetrahydrofuran-3,4-diol as a solid (Compound 20, 35 mg, 31% yield).

¹H NMR (400 MHz, MeOD-d4) δ 8.06 (s, 1H), 7.32 (d, J=3.1 Hz, 1H), 6.61 (d, J=2.7 Hz, 1H), 5.98 (d, J=7.7 Hz, 1H), 4.84-4.83 (m, 1H), 4.74 (d, J=9.8 Hz, 1H), 4.60 (dd, J=20.2, 9.8 Hz, 1H), 4.45 (d, J=9.8 Hz, 1H), 4.32 (d, J=5.1 Hz, 1H), 3.76 (s, 2H).

¹⁹F NMR (377 MHz, MeOD-d4) δ −236.74.

LCMS(ESI): m/z 299.0 [M+H]⁺.

### Example 6

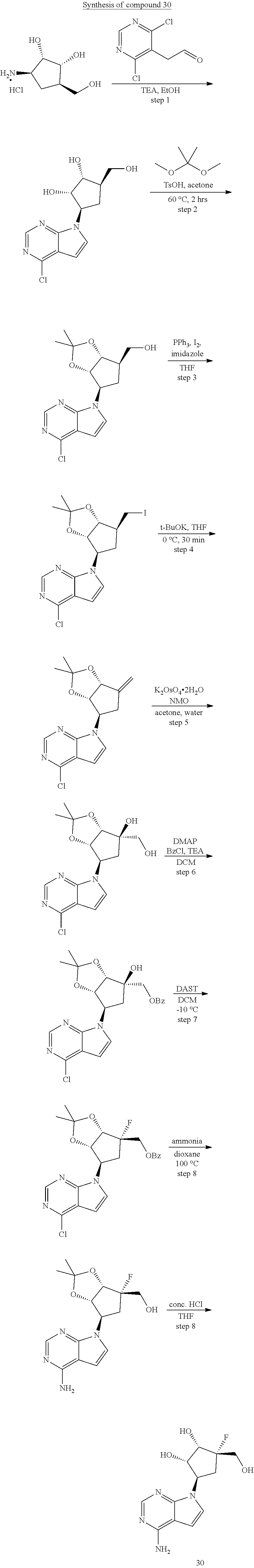

Step 1: Synthesis of (1R,2S,3R,5R)-3-(4-chloro-7H-pyrrolo[2,3-d]pyrimidin-7-yl)-5-(hydroxymethyl)cyclopentane-1,2-diol: To a solution of 2-(4,6-dichloropyrimidin-5-yl)acetaldehyde (1.91 g, 10.0 mmol) and (1R,2S,3R,5R)-3-amino-5-(hydroxymethyl)cyclopentane-1,2-diol hydrochloride (1.84 g, 10.0 mmol) in EtOH (50 mL) was added TEA (3.03 mg, 30.0 mmol) and the reaction mixture was stirred at 80° C. for 24 hrs. After completion of the reaction, the mixture was concentrated under vacuum. The residue was dissolved in sat. NaHCO₃ solution, and then extracted with EtOAc. The combined organic layers were washed with brine, dried over Na₂SO₄, and then filtered. The filtrate was concentrated under reduced pressure to afford the crude (1R,2S,3R,5R)-3-(4-chloro-7H-pyrrolo[2,3-d]pyrimidin-7-yl)-5-(hydroxymethyl)cyclopentane-1,2-diol (2.83 g) as a yellow gum, which was used for next step without further purification. LCMS(ESI): m/z=284.1 [M+H]⁺.

Step 2: Synthesis of [(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]methanol: To a solution of (1R,2S,3R,5R)-3-(4-chloro-7H-pyrrolo[2,3-d]pyrimidin-7-yl)-5-(hydroxymethyl)cyclopentane-1,2-diol (2.83 g, 10.0 mmol) and 2,2-dimethoxypropane (2.08 g, 20 mmol) in acetone (60 mL) was added 4-methylbenzenesulfonic acid hydrate (194 mg, 1 mmol). The mixture was stirred at r.t. for 2 h and then refluxed for 24 h. After completion of the reaction, the reaction was quenched by Et₃N, and then concentrated under reduced pressure. The residue was treated with sat. NaHCO₃ solution and brine. The resulting mixture was extracted with EtOAc. The combined organic layers were washed with brine, dried over Na₂SO₄, and then filtered. The filtrate was concentrated under reduced pressure. The residue was purified by column chromatography to afford [(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]methanol (3.0 g, 93% yield) as a yellow solid. LCMS(ESI): m/z=324.1 [M+H]⁺.

Step 3: Synthesis of 7-[(3aS,4R,6S,6aR)-6-(iodomethyl)-2,2-dimethyl-4,5,6,6a-tetrahydro-3aH-cyclopenta[d][1,3]dioxol-4-yl]-4-chloropyrrolo[2,3-d]pyrimidine: To a solution of PPh₃ (4.86 g, 9.29 mmol) and imidazole (1.94 g, 19.51 mmol) in THE (30 mL) was added 1₂ (4.72 g, 18.57 mmol). The mixture was stirred at room temperature for 15 min under N₂, and then a solution of [(3aS,4R,6R,6aR)-4-(4-chloropyrrolo[2,3-d]pyrimidin-7-yl)-2,2-dimethyl-4,5,6,6a-tetrahydro-3aH-cyclopenta[d][1,3]dioxol-6-yl]methanol (3000 mg, 9.29 mmol) in THE (20 mL) was added. The mixture was stirred at room temperature for 1.5 h under N₂. After completion of the reaction, the reaction was quenched with sat. Na₂S₂O₃ solution, and then extracted with DCM. The combined organic layers were washed with brine, dried over Na₂SO₄, and then filtered. The filtrate was concentrated in vacuum. The residue was purified by column chromatography to afford 7-[(3aS,4R,6S,6aR)-6-(iodomethyl)-2,2-dimethyl-4,5,6,6a-tetrahydro-3aH-cyclopenta[d][1,3]dioxol-4-yl]-4-chloropyrrolo[2,3-d]pyrimidine (3.0 g, 69% yield) as a yellow solid. LCMS(ESI): m/z=434.1 [M+H]⁺.

Step 4: Synthesis of 4-chloro-7-((3aS,4R,6aR)-2,2-dimethyl-6-methylenetetrahydro-4H-cyclopenta[d][1,3]dioxol-4-yl)-7H-pyrrolo[2,3-d]pyrimidine: To a solution of 7-[(3aS,4R,6S,6aR)-6-(iodomethyl)-2,2-dimethyl-4,5,6,6a-tetrahydro-3aH-cyclopenta[d][1,3]dioxol-4-yl]-4-chloropyrrolo[2,3-d]pyrimidine (3000 mg, 6.93 mmol) in THE (20 mL) was added 1M t-BuOK in THE (6.93 mL) at 0° C. The reaction mixture was stirred at 0° C. for 30 mins. After completion of the reaction, the reaction was quenched with sat. aq. NH₄Cl. The mixture was extracted with EtOAc. The separated organic layers were combined and washed with brine, dried over anhydrous Na₂SO₄ and concentrated under reduce pressure. The residue was purified by silica gel chromatography to afford 4-chloro-7-((3aS,4R,6aR)-2,2-dimethyl-6-methylenetetrahydro-4H-cyclopenta[d][1,3]dioxol-4-yl)-7H-pyrrolo[2,3-d]pyrimidine (2.12 g, 94% yield) as colorless foam. LCMS(ESI): m/z=306.1 [M+H]⁺.

Step 5: Synthesis of (3aS,4R,6R,6aS)-6-(4-chloropyrrolo[2,3-d]pyrimidin-7-yl)-4-(hydroxymethyl)-2,2-dimethyl-3a,5,6,6a tetrahydrocyclopenta[d][1,3]dioxol-4-ol: To a mixture of 4-chloro-7-((3aS,4R,6aR)-2,2-dimethyl-6-methylenetetrahydro-4H-cyclopenta[d][1,3]dioxol-4-yl)-7H-pyrrolo[2,3-d]pyrimidine. (1300 mg, 4.024 mmol) and NMO (943 mg, 8.06 mmol) in a mixed solvent of acetone (26 mL) and H₂O (5.2 mL) was added K₂OsO₄·2H₂O (30.3 mg, 0.098 mmol). The mixture was stirred at rt for 20 h. After completion of the reaction, the mixture was concentrated under reduced pressure. The residue was purified by silica gel chromatography to afford (3aS,4R,6R,6aS)-6-(4-chloropyrrolo[2,3-d]pyrimidin-7-yl)-4-(hydroxymethyl)-2,2-dimethyl-3a,5,6,6a tetrahydrocyclopenta[d][1,3]dioxol-4-ol (1300 mg, 95% yield) as a light yellow foam. LCMS(ESI): m/z=340.0 [M+H]⁺.

Step 6: Synthesis of [(3aS,4R,6R,6aS)-6-(4-chloropyrrolo[2,3-d]pyrimidin-7-yl)-4-hydroxy-2,2-dimethyl-3a,5,6,6a tetrahydrocyclopenta[d][1,3]dioxol-4-yl]methyl benzoate: To a solution of (3aS,4R,6R,6aS)-6-(4-chloropyrrolo[2,3-d]pyrimidin-7-yl)-4-(hydroxymethyl)-2,2-dimethyl-3a,5,6,6a tetrahydrocyclopenta[d][1,3]dioxol-4-ol (1300 mg, 3.83 mmol), DMAP (46.7 mg, 0.383 mmol), and Et₃N (581 mg, 5.75 mmol) in DCM (35 mL) was added benzoyl chloride (1079 mg, 7.67 mmol) dropwise at rt. The mixture was stirred at rt for 3 h. After completion of the reaction, the mixture was concentrated under reduced pressure. The residue was purified by silica gel chromatography to afford [(3aS,4R,6R,6aS)-6-(4-chloropyrrolo[2,3-d]pyrimidin-7-yl)-4-hydroxy-2,2-dimethyl-3a,5,6,6a tetrahydrocyclopenta[d][1,3]dioxol-4-yl]methyl benzoate (1.3 g, 94% yield) as a white foam. LCMS(ESI): m/z=444.1 [M+H]⁺.

Step 7: Synthesis of ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methyl benzoate: To a solution of ((3aS,4R,6R,6aS)-6-(4-chloro-7H-pyrrolo[2,3-d]pyrimidin-7-yl)-4-hydroxy-2,2-dimethyltetrahydro-4H-cyclopenta[d][1,3]dioxol-4-yl)methyl benzoate (600 mg, 1.355 mmol) in anhydrous DCM (30 mL) was added DAST (436.5 mg, 2.71 mmol) at 0° C. The reaction mixture was stirred at 0° C. for 1 hr. The mixture was quenched with saturated NaHCO₃ aqueous solution and extracted with DCM. The separated organic layers were combined and washed with brine, dried over anhydrous Na₂SO₄ and concentrated under reduced pressure. The residue was purified by chromatography on silica gel to afford ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methyl benzoate (62 mg, 10%) as a white foam. LCMS(ESI): m/z=446.2 [M+H]⁺.

Step 8: Synthesis of ((3aS,4S,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)methanol: To a solution of ((3aS,4S,6R,6aS)-6-(4-chloro-7H-pyrrolo[2,3-d]pyrimidin-7-yl)-4-fluoro-2,2-dimethyltetrahydro-4H-cyclopenta[d][1,3]dioxol-4-yl)methyl benzoate (39 mg, 0.0876 mmol) in dioxane (2 mL) was added conc. ammonia (2 mL). The reaction was then stirred at 100° C. for 16 hrs. The solvent was removed in vacuum to give ((3aS,4S,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)methanol (20 mg, 71%) as a pale yellow foam. LCMS: m/z=323.2 [M+H]⁺.

Step 9: Synthesis of (1S,2S,3S,5R)-5-(4-amino-7H-pyrrolo[2,3-d]pyrimidin-7-yl)-3-fluoro-3-(hydroxymethyl)cyclopentane-1,2-diol (9): To a solution of ((3aS,4S,6R,6aS)-6-(4-amino-7H-pyrrolo[2,3-d]pyrimidin-7-yl)-4-fluoro-2,2-dimethyltetrahydro-4H-cyclopenta[d][1,3]dioxol-4-yl)methanol (2 mg, 0.0062 mmol) in THE (1 mL) was added 4M HCl (0.5 mL). The reaction was then stirred at 0° C. for 1 hr. After completion of the reaction, the solvent was removed in vacuum and purified by reverse phase prep-HPLC to give (1S,2S,3S,5R)-5-(4-amino-7H-pyrrolo[2,3-d]pyrimidin-7-yl)-3-fluoro-3-(hydroxymethyl)cyclopentane-1,2-diol (Compound 9, 1.5 mg, 83%) as a white foam. ¹H NMR (400 MHz, Methanol-d₄) δ 8.21 (s, 1H), 7.49 (d, J=3.7 Hz, 1H), 6.85 (d, J=3.6 Hz, 1H), 5.22 (dd, J=17.0, 9.7 Hz, 1H), 4.47 (t, J=6.7 Hz, 1H), 4.21 (dd, J=13.0, 6.2 Hz, 1H), 3.88-3.69 (m, 2H), 2.52 (ddd, J=20.0, 14.8, 9.5 Hz, 1H), 2.42-2.25 (m, 1H). LCMS(ESI): m/z=283.0 [M+H]⁺.

### Example 7

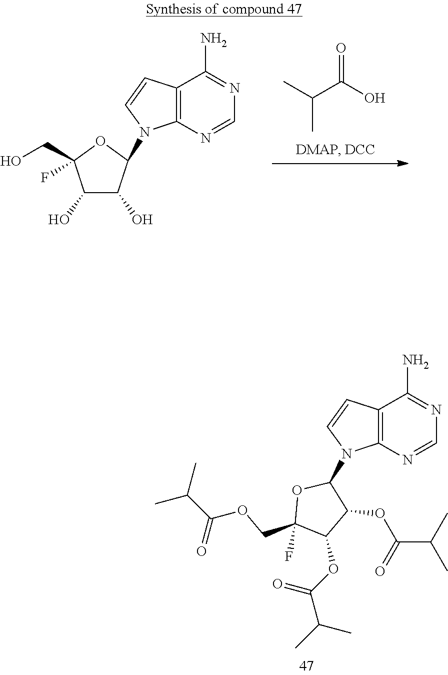

Synthesis of (2S,3S,4R,5R)-5-(4-amino-7H-pyrrolo[2,3-d]pyrimidin-7-yl)-2-fluoro-2-((isobutyryloxy)methyl)tetrahydrofuran-3,4-diyl bis(2-methylpropanoate) (47): To a solution of (2S,3S,4R,5R)-5-(4-amino-7H-pyrrolo[2,3-d]pyrimidin-7-yl)-2-fluoro-2-(hydroxymethyl) tetrahydrofuran-3,4-diol (Compound 1, 500 mg, 1.76 mmol) in dichloromethane (10 mL) was added isobutyric acid (511 mg, 5.81 mmol), 4-dimethylaminopyridine (65 mg, 0.53 mmol) and dicyclohexylcarbodiimide (1.2 g, 5.81 mmol) at room temperature. The mixture was stirred for 2 hours. After the reaction was completed, the insoluble solids were removed by filtration and the filtrate was concentrated under reduced pressure. The residue was purified by Pre-HPLC to afford to afford (2S,3S,4R,5R)-5-(4-amino-7H-pyrrolo[2,3-d]pyrimidin-7-yl)-2-fluoro-2((isobutyryloxy)methyl)tetrahydrofuran-3,4-diyl bis(2-methylpropanoate) (Compound 47,450 mg, 52% yield) as a white solid. ¹H NMR (300 MHz, DMSO-d₆) δ 8.09 (s, 1H), 7.37 (d, J=3.6 Hz, 1H), 7.17 (s, 2H), 6.64 (d, J=3.6 Hz, 1H), 6.50 (d, J=1.8 Hz, 1H), 6.25 (dd, J=19.1, 7.0 Hz, 1H), 5.93 (dd, J=7.0, 2.1 Hz, 1H), 4.42-4.23 (m, 2H), 2.63 (dt, J=13.9, 6.9 Hz, 2H), 2.48-2.40 (m, 1H), 1.15 (d, J=6.9 Hz, 6H), 1.13-1.02 (m, 9H), 0.97 (d, J=7.0 Hz, 3H). LCMS(ESI): m/z 495.3 [M+H]⁺.

Procedure analogous to those for the synthesis of compound 47 were used for the synthesis of compound 46 and 73.

### Example 8

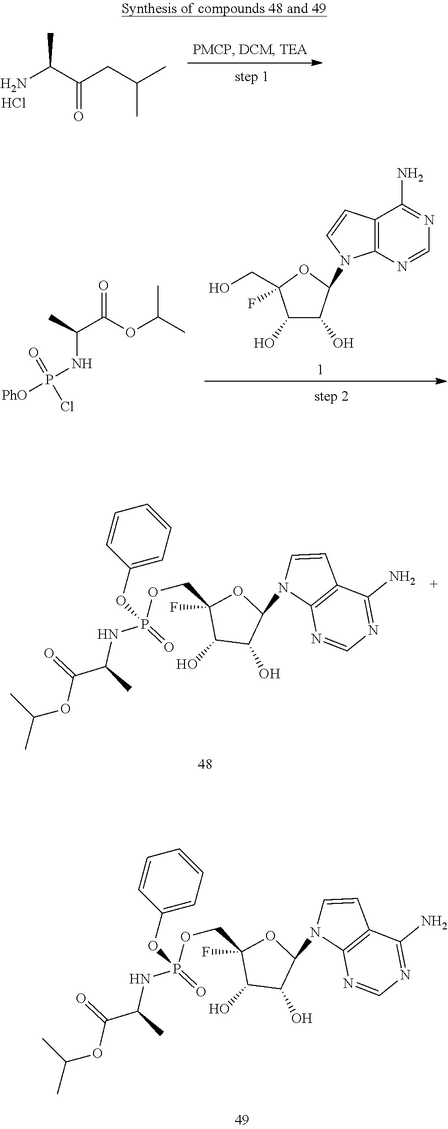

Step 1: Synthesis of isopropyl (2S)-2-{[chloro(phenoxy)phosphoryl]amino}propanoate: A solution of isopropyl (2S)-2-aminopropanoate hydrochloride (3 g, 17.8 mmol) in DCM (60 mL) was treated with phenoxyphosphonoyl dichloride (4.15 g, 19.6 mmol) at −78° C. for 30 min under nitrogen atmosphere followed by the addition of TEA (3.62 g, 35.7 mmol) dropwise at −78° C. The resulting mixture was stirred at room temperature for additional 1 h. The resulting mixture was concentrated under reduced pressure. The resulting mixture was diluted with MTBE (30 mL). The resulting mixture was filtered, the filter cake was washed with MTBE (2×10 mL). The filtrate was concentrated under reduced pressure. The crude product-isopropyl (chloro(phenoxy)phosphoryl)-L-alaninate (4.2 g) was used in the next step directly without further purification. LCMS(ESI) m/z 306.1 [M+H]⁺.

Step 2: Synthesis of isopropyl (2S)-2-({[(2S,3S,4R,5R)-5-{4-aminopyrrolo[2,3-d]pyrimidin-7-yl}-2-fluoro-3,4-dihydroxyoxolan-2-yl]methoxy(phenoxy)phosphoryl}amino)propanoate (48 and 49): To a stirred solution of (2S,3S,4R,5R)-5-{4-aminopyrrolo[2,3-d]pyrimidin-7-yl}-2-fluoro-2-(hydroxymethyl)oxolane-3,4-diol (Compound 1, 800 mg, 2.81 mmol) in THE (10 mL) and trimethyl phosphate (1 mL) were added 1-methyl-1H-imidazole (1.27 g, 15.4 mmol) and a solution of isopropyl (2S)-2-{[chloro(phenoxy)phosphoryl]amino}propanoate (2.15 g, 7.03 mmol) in THE (4 mL) dropwise at 0° C. under nitrogen atmosphere. The resulting mixture was stirred at room temperature for additional 1 h. The reaction was quenched with H₂O at 0° C. The resulting mixture was extracted with EtOAc. The combined organic layers were washed with brine (1×20 mL), dried over anhydrous Na₂SO₄. After filtration, the filtrate was concentrated under reduced pressure. The crude product was purified by Prep-HPLC and Prep-SFC to afford isopropyl (2S)-2-({[(2S,3S,4R,5R)-5-{4-aminopyrrolo[2,3-d]pyrimidin-7-yl}-2-fluoro-3,4-dihydroxyoxolan-2-yl]methoxy(phenoxy)phosphoryl}amino)propanoate (Compound 48, 2.4 mg, the second peak in Prep-SFC) and isopropyl (2S)-2-({[(2S,3S,4R,5R)-5-{4-aminopyrrolo[2,3-d]pyrimidin-7-yl}-2-fluoro-3,4-dihydroxyoxolan-2-yl]methoxy(phenoxy)phosphoryl}amino)propanoate (Compound 49, 5.3 mg, the first peak in Prep-SFC) as a white solid. LCMS m/z [M+H]⁺ 554.1.

Compound 48: ¹H NMR (300 MHz, DMSO-d6) δ 8.08 (s, 1H), 7.32 (t, J=7.7 Hz, 2H), 7.24 (d, J=3.6 Hz, 1H), 7.15 (m, 5H), 6.62 (d, J=3.7 Hz, 1H), 6.35 (d, J=3.2 Hz, 1H), 6.06 (dd, J=13.1, 10.1 Hz, 1H), 5.81 (d, J=5.6 Hz, 1H), 5.40 (d, J=8.5 Hz, 1H), 4.82 (m, 1H), 4.58 (m, 1H), 4.46 (m, 1H), 4.14 (m, 2H), 3.83-3.65 (m, 1H), 1.22-0.98 (m, 9H). ¹⁹F NMR (282 MHz, DMSO-d6) δ −120.63.

Compound 49: ¹H NMR (300 MHz, DMSO-d6) δ 8.08 (s, 1H), 7.34 (t, J=7.8 Hz, 2H), 7.24-7.08 (m, 6H), 6.63 (d, J=3.7 Hz, 1H), 6.36 (d, J=3.0 Hz, 1H), 6.03 (dd, J=13.4, 9.9 Hz, 1H), 5.82 (d, J=5.4 Hz, 1H), 5.43 (d, J=8.7 Hz, 1H), 4.83 (m, 1H), 4.58 (m, 1H), 4.46 (s, 1H), 4.17 (m, 2H), 3.69 (m, 1H), 1.13 (d, J=5.8 Hz, 9H). ¹⁹F NMR (282 MHz, DMSO-d6) δ −120.27.

Procedure analogous to those for the synthesis of compound 48 and 49 were used for the synthesis of compound 74.

### Example 9

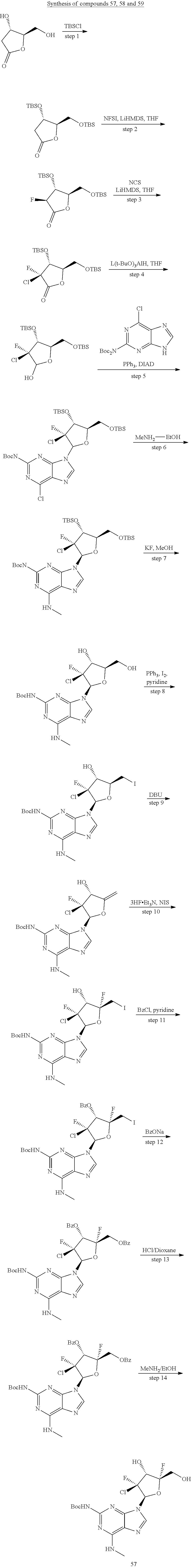

Step 1: Synthesis of (4S,5R)-4-[(tert-butyldimethylsilyl)oxy]-5-{[(tert-butyldimethylsilyl)oxy]methyl}oxolan-2-one: To a solution of deoxyribonolactone (50 g, 378.458 mmol) in DMF (500 mL) was added imidazole (64.41 g, 946.1 mmol) and DMAP (2.31 g, 18.92 mmol) and TBSCl (119.8 g, 794.8 mmol) at 0° C. The mixture was stirred at 20° C. for 12 h. The mixture was quenched with NaHCO₃ (500 mL) and extracted with EtOAc (3×500 mL), dried over anhydrous Na₂SO₄. After filtration, the filtrate was concentrated. The residue was purified by silica gel column chromatography to afford (4S,5R)-4-[(tert-butyldimethylsilyl)oxy]-5-{[(tert-butyldimethylsilyl)oxy]methyl}oxolan-2-one (110 g, 81%) as a white solid. ¹H NMR (300 MHz, Chloroform-d) δ 4.52 (m, 1H), 4.35 (m, 1H), 3.87-3.74 (m, 2H), 2.84 (dd, J=17.6, 6.7 Hz, 1H), 2.40 (dd, J=17.6, 2.6 Hz, 1H), 0.91 (s, 18H), 0.14-0.06 (m, 12H).

Step 2: Synthesis of (3S,4R,5R)-4-[(Tert-butyldimethylsilyl)oxy]-5-{[(tert-butyldimethylsilyl)oxy]methyl}-3-fluorooxolan-2-one: To a solution of (4S,5R)-4-[(tert-butyldimethylsilyl)oxy]-5-{[(tert-butyldimethylsilyl)oxy]methyl}oxolan-2-one (60 g, 166.4 mmol) and NFSI (65.58 g, 208.0 mmol) in THE (1200 mL) was added LiHMDS (232.9 mL, 233.0 mmol) at −78° C. The mixture was stirred at −78° C. for 2 h. The reaction was quenched with NH₄Cl at −78° C. The resulting mixture was extracted with EtOAc. The combined organic layers were washed with brine (300 mL), dried over anhydrous Na₂SO₄. After filtration, the filtrate was concentrated under reduced pressure. The residue was purified by silica gel column chromatography to afford (3S,4R,5R)-4-[(tert-butyldimethylsilyl)oxy]-5-{[(tert-butyldimethylsilyl)oxy]methyl}-3-fluorooxolan-2-one (30 g, 48%) as a colorless oil.

Step 3: Synthesis of (3S,4R,5R)-4-[(Tert-butyldimethylsilyl)oxy]-5-{[(tert-butyldimethylsilyl)oxy]methyl}-3-chloro-3-fluorooxolan-2-one: To a solution of (3S,4R,5R)-4-[(tert-butyldimethylsilyl)oxy]-5-{[(tert-butyldimethylsilyl)oxy]methyl}-3-fluorooxolan-2-one (28 g, 7.98 mmol) and NCS (23.52 g, 147.90 mmol) in THE (110 mL) was added LiHMDS (184.9 mL, 174.9 mmol) at −78° C. The mixture was stirred at −78° C. for 2 h. The reaction was quenched with sat. NH₄Cl (aq.) at −78° C. The resulting mixture was extracted with EtOAc. The combined organic layers were washed with brine and dried over anhydrous Na₂SO₄. After filtration, the filtrate was concentrated under reduced pressure. The residue was purified by silica gel column chromatography to afford (3S,4R,5R)-4-[(tert-butyldimethylsilyl)oxy]-5-{[(tert-butyldimethylsilyl)oxy]methyl}-3-chloro-3-fluorooxolan-2-one (12 g, 39%) as a yellow oil. ¹H NMR (300 MHz, Chloroform-d) δ 4.60 (dd, J=12.0, 5.7 Hz, 1H), 4.40-4.33 (m, 1H), 4.00 (dd, J=12.1, 3.9 Hz, 1H), 3.90-3.83 (m, 1H), 0.95 (s, 9H), 0.91 (s, 9H), 0.23 (s, 3H), 0.19 (s, 3H), 0.11 (d, J=2.7 Hz, 6H).

Step 4: Synthesis of (3S,4R,5R)-4-[(Tert-butyldimethylsilyl)oxy]-5-{[(tert-butyldimethylsilyl)oxy]methyl}-3-chloro-3-fluorooxolan-2-ol: To a solution of (3S,4R,5R)-4-[(tert-butyldimethylsilyl)oxy]-5-{[(tert-butyldimethylsilyl)oxy]methyl}-3-chloro-3-fluorooxolan-2-one (12 g, 29.05 mmol) in THE (120 mL) was added Li(t-BuO)₃AlH (92.63 mL, 82.63 mmol) at 0° C. The mixture was stirred at 20° C. for 2 h and then was quenched with sat. NH₄Cl (aq.) at 0° C. The resulting mixture was extracted with EA. The combined organic layers were washed with brine and dried over anhydrous Na₂SO₄. After filtration, the filtrate was concentrated under reduced pressure. The residue was purified by silica gel column chromatography to afford (3S,4R,5R)-4-[(tert-butyldimethylsilyl)oxy]-5-{[(tert-butyldimethylsilyl)oxy]methyl}-3-chloro-3-fluorooxolan-2-ol (12.2 g, 100%) as a yellow oil.

Step 5: Synthesis of tert-Butyl N-(tert-butoxycarbonyl)-N-{9-[(2R,3S,4R,5R)-4-[(tert-butyldimethylsilyl)oxy]-5-{[(tert-butyldimethylsilyl)oxy]methyl}-3-chloro-3-fluorooxolan-2-yl]-6-chloropurin-2-yl}carbamate: To a solution of (3S,4R,5R)-4-[(tert-butyldimethylsilyl)oxy]-5-{[(tert-butyldimethylsilyl)oxy]methyl}-3-chloro-3-fluorooxolan-2-ol (11.25 g, 27.1 mmol) and tert-butyl N-(tert-butoxycarbonyl)-N-(6-chloro-9H-purin-2-yl)carbamate (12 g, 32.53 mmol) in THE (320 mL) was added PPh₃ (10.68 g, 40.65 mmol). Cooled to 0° C., DIAD (6.6 g, 37.95 mmol) was added and the mixture was stirred at 70° C. for 1 h. The resulting mixture was extracted with EtOAc. The combined organic layers were washed with brine and dried over anhydrous Na₂SO₄. After filtration, the filtrate was concentrated under reduced pressure. The residue was purified by silica gel column chromatography to afford tert-butyl N-(tert-butoxycarbonyl)-N-{9-[(2R,3S,4R,5R)-4-[(tert-butyldimethylsilyl)oxy]-5-{[(tert-butyldimethylsilyl)oxy]methyl}-3-chloro-3-fluorooxolan-2-yl]-6-chloropurin-2-yl}carbamate (4.6 g, 22%) as a white solid. LCMS(ESI): m/z 766.7 [M+H]⁺.

Step 6: Synthesis of tert-butyl N-(tert-butoxycarbonyl)-N-{9-[(2R,3S,4R,5R)-4-[(tert-butyldimethylsilyl)oxy]-5-{[(tert-butyldimethylsilyl)oxy]methyl}-3-chloro-3-fluorooxolan-2-yl]-6-(methylamino)purin-2-yl}carbamate: To a solution of tert-butyl N-(tert-butoxycarbonyl)-N-{9-[(2R,3S,4R,5R)-4-[(tert-butyldimethylsilyl)oxy]-5-{[(tert-butyldimethylsilyl)oxy]methyl}-3-chloro-3-fluorooxolan-2-yl]-6-chloropurin-2-yl}carbamate (4.6 g, 6.01 mmol, 1 equiv) in EtOH (45 mL) was added methylamine (33% in ethanol). The mixture was stirred at 60° C. for 2 h and the resulting mixture was concentrated under vacuum. The residue was purified by silica gel column chromatography to afford tert-butyl N-(tert-butoxycarbonyl)-N-{9-[(2R,3S,4R,5R)-4-[(tert-butyldimethylsilyl)oxy]-5-{[(tert-butylimethylsilyl)oxy]methyl}-3-chloro-3-fluorooxolan-2-yl]-6-(methylamino)purin-2-yl}carbamate (4 g, 98%) as an off-white solid. LCMS(ESI): m/z 661.5 [M+H]⁺.

Step 7: Synthesis of tert-butyl N-{9-[(2R,3S,4R,5R)-3-chloro-3-fluoro-4-hydroxy-5-(hydroxymethyl)oxolan-2-yl]-6-(methylamino)purin-2-yl}carbamate: To a solution of tert-butyl N-{9-[(2R,3S,4R,5R)-4-[(tert-butyldimethylsilyl)oxy]-5-{[(tert-butyldimethylsilyl)oxy]methyl}-3-chloro-3-fluorooxolan-2-yl]-6-(methylamino)purin-2-yl}carbamate (4 g, 6.048 mmol) in MeOH (80 mL) was added KF (3.51 g, 60.48 mmol). The mixture was stirred at 60° C. for 6 h and the resulting mixture was concentrated under vacuum. The residue was purified by silica gel column chromatography to afford tert-butyl N-{9-[(2R,3S,4R,5R)-3-chloro-3-fluoro-4-hydroxy-5-(hydroxymethyl)oxolan-2-yl]-6-(methylamino)purin-2-yl}carbamate (2.5 g, 96%) as a white solid. LCMS(ESI): m/z 433.0 [M+H]⁺.

Step 8: Synthesis of tert-Butyl N-{9-[(2R,3S,4R,5S)-3-chloro-3-fluoro-4-hydroxy-5-(iodomethyl)oxolan-2-yl]-6-(methylamino)purin-2-yl}carbamate: To a solution of tert-butyl N-{9-[(2R,3S,4R,5R)-3-chloro-3-fluoro-4-hydroxy-5-(hydroxymethyl)oxolan-2-yl]-6-(methylamino)purin-2-yl}carbamate (2.5 g, 5.776 mmol) in THE (40 mL) was added PPh₃ (4.545 g, 17.33 mmol) and pyridine (4.569 g, 57.76 mmol) and 12 (2.932 g, 11.55 mmol). The mixture was stirred at 20° C. for 12 h. The resulting mixture was concentrated under vacuum. The residue was purified by silica gel column chromatography, eluted with PE/THF (2/1) to afford tert-butyl N-{9-[(2R,3S,4R,5S)-3-chloro-3-fluoro-4-hydroxy-5-(iodomethyl)oxolan-2-yl]-6-(methylamino)purin-2-yl}carbamate (3 g, 96%) as a yellow oil. LCMS(ESI): m/z 543.0 [M+H]⁺.

Step 9: Synthesis of tert-Butyl N-{9-[(2R,3S,4R)-3-chloro-3-fluoro-4-hydroxy-5-methylideneoxolan-2-yl]-6-(methylamino)purin-2-yl}carbamate: To a solution of tert-butyl N-{9-[(2R,3S,4R,5S)-3-chloro-3-fluoro-4-hydroxy-5-(iodomethyl)oxolan-2-yl]-6-(methylamino)purin-2-yl}carbamate (3 g, 5.528 mmol) in THE (50 mL) was added DBU (3.37 g, 22.11 mmol). The mixture was stirred at 20° C. for 7 h and the resulting mixture was extracted with EtOAc. The combined organic layers were washed with brine and dried over anhydrous Na₂SO₄. After filtration, the filtrate was concentrated under reduced pressure. The crude product was purified by Prep-HPLC to afford tert-butyl N-{9-[(2R,3S,4R)-3-chloro-3-fluoro-4-hydroxy-5-methylideneoxolan-2-yl]-6-(methylamino)purin-2-yl}carbamate (830 mg, 36%) as a white solid. LCMS(ESI): m/z 415.2 [M+H]⁺.

Step 10: Synthesis of tert-Butyl N-{9-[(2R,3S,4R,5R)-3-chloro-3,5-difluoro-4-hydroxy-5-(iodomethyl)oxolan-2-yl]-6-(methylamino)purin-2-yl}carbamate: To a solution of tert-butyl N-{9-[(2R,3S,4R)-3-chloro-3-fluoro-4-hydroxy-5-methylideneoxolan-2-yl]-6-(methylamino)purin-2-yl}carbamate (600 mg, 1.446 mmol) in MeCN (6 mL) was added TEA 3HF (349.8 mg, 2.169 mmol) at 0° C. The mixture was stirred for 10 min. NIS (488.1 mg, 2.169 mmol) was added at 0° C. and the mixture was allowed to warm to room temperature and stirred for 1 hour. After completion of reaction, reaction was quenched slowly with ice cold water and extracted with EtOAc. The resulting mixture was concentrated under reduced pressure and the crude product tert-butyl N-{9-[(2R,3S,4R,5R)-3-chloro-3,5-difluoro-4-hydroxy-5-(iodomethyl)oxolan-2-yl]-6-(methylamino)purin-2-yl}carbamate (1.1 g, crude) was used in the next step directly without further purification. LCMS(ESI): m/z 561.0 [M+H]⁺.

Step 11: Synthesis of (2R,3R,4S,5R)-5-{2-[(tert-Butoxycarbonyl)amino]-6-(methylamino)purin-9-yl}-4-chloro-2,4-difluoro-2-(iodomethyl)oxolan-3-yl benzoate: To a solution of tert-butyl N-{9-[(2R,3S,4R,5R)-3-chloro-3,5-difluoro-4-hydroxy-5-(iodomethyl)oxolan-2-yl]-6-(methylamino)purin-2-yl}carbamate (1.1 g, 1.96 mmol) in pyridine (8 mL) was added benzoyl chloride (1378.8 mg, 9.80 mmol) at 0° C. The mixture was stirred for 45 min. After completion of the reaction, mixture was quenched with aqueous ammonium chloride solution and extracted with ethyl acetate. The combined organic layer was dried over anhydrous sodium sulfate, filtered and concentrated under reduced pressure. The crude material was purified by column chromatography to afford (2R,3R,4S,5R)-5-{2-[(tert-butoxycarbonyl)amino]-6-(methylamino)purin-9-yl}-4-chloro-2,4-difluoro-2-(iodomethyl)oxolan-3-yl benzoate (600 mg, 46%). LCMS(ESI): m/z 665.1 [M+H]⁺.

Step 12: Synthesis of [(2S,3R,4S,5R)-3-(Benzoyloxy)-5-{2-[(tert-butoxycarbonyl)amino]-6-(methylamino)purin-9-yl}-4-chloro-2,4-difluorooxolan-2-yl]methyl benzoate: To a solution of (2R,3R,4S,5R)-5-{2-[(tert-butoxycarbonyl)amino]-6-(methylamino)purin-9-yl}-4-chloro-2,4-difluoro-2-(iodomethyl)oxolan-3-yl benzoate (600 mg, 0.903 mmol) in DMSO (10 mL) was added sodium benzoate (1.040 g, 7.224 mmol). The mixture was stirred at 100° C. for 12 h and the resulting mixture was extracted with EtOAc. The combined organic layers were washed with brine and dried over anhydrous Na₂SO₄. After filtration, the filtrate was concentrated under reduced pressure. The residue was purified by silica gel column chromatography to afford [(2S,3R,4S,5R)-3-(benzoyloxy)-5-{2-[(tert-butoxycarbonyl)amino]-6-(methylamino)purin-9-yl}-4-chloro-2,4-difluorooxolan-2-yl]methyl benzoate (400 mg, 67%) as a yellow oil. LCMS(ESI): m/z 659.2 [M+H]⁺.

Step 13: Synthesis of [(2S,3R,4S,5R)-5-[2-Amino-6-(methylamino)purin-9-yl]-3-(benzoyloxy)-4-chloro-2,4-difluorooxolan-2-yl]methyl benzoate: A solution of [(2S,3R,4S,5R)-3-(benzoyloxy)-5-{2-[(tert-butoxycarbonyl)amino]-6-(methylamino)purin-9-yl}-4-chloro-2,4-difluorooxolan-2-yl]methyl benzoate (400 mg, 0.304 mmol) and HCl (1 mL, 4.0 mmol) in DCM (3 mL) was stirred at 20° C. for 4 h. The mixture was basified to pH=7 with saturated NaHCO₃ (aq.). The resulting mixture was extracted with EtOAc. The combined organic layers were washed with brine and dried over anhydrous Na₂SO₄. After filtration, the filtrate was concentrated under reduced pressure to yield [(2S,3R,4S,5R)-5-[2-amino-6-(methylamino)purin-9-yl]-3-(benzoyloxy)-4-chloro-2,4-difluorooxolan-2-yl]methyl benzoate (300 mg, 89%) as a yellow oil. LCMS(ESI): m/z 559.2 [M+H]⁺.

Step 14: Synthesis of (2S,3R,4S,5R)-5-[2-Amino-6-(methylamino)purin-9-yl]-4-chloro-2,4-difluoro-2-(hydroxymethyl)oxolan-3-ol (57): A solution of tert-butyl N-{9-[(2R,3S,4R,5S)-3-chloro-3,5-difluoro-4-hydroxy-5-(hydroxymethyl)oxolan-2-yl]-6-(methylamino)purin-2-yl}carbamate (280 mg, 0.621 mmol) in methylamine (33% in ethanol) (4 mL) was stirred at 20° C. for 2 h. The resulting mixture was concentrated under vacuum. The crude product was purified by Prep-HPLC to afford (2S,3R,4S,5R)-5-[2-amino-6-(methylamino)purin-9-yl]-4-chloro-2,4-difluoro-2-(hydroxymethyl)oxolan-3-ol (Compound 57, 95 mg, 51%) as a white solid. LCMS(ESI): m/z 351.0 [M+H]⁺.

Procedure analogous to those for the synthesis of compound 57 were used for the synthesis of compound 56, 60, 62, 63, 66, 68, 70 and 71 et al.

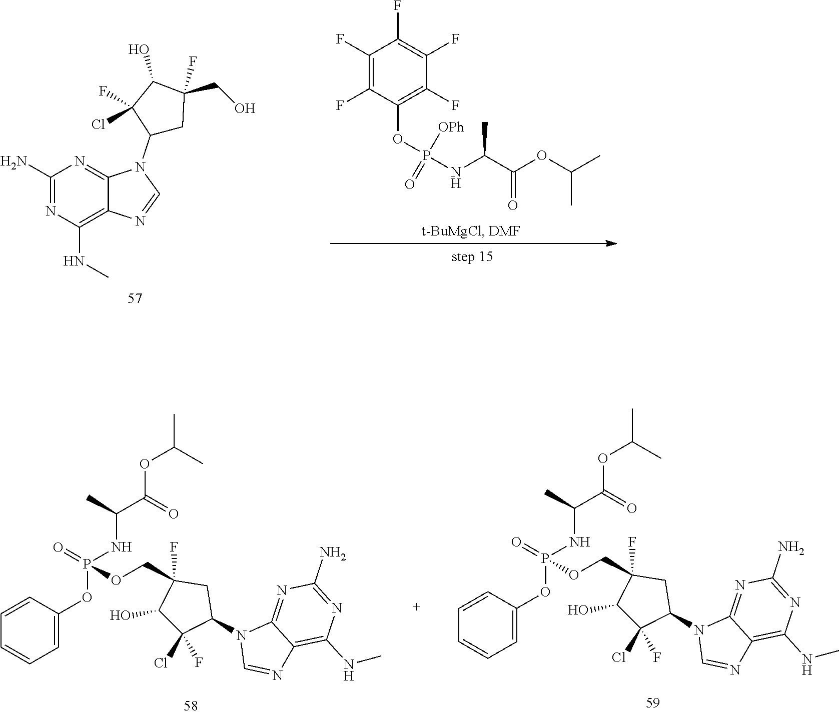 [SMILES: CNc1nc(N)nc2c1ncn2C1C[C@](F)(CO)[C@@H](O)[C@]1(F)Cl ; CC(C)OC(=O)[C@H](C)NP(=O)(Oc1ccccc1)Oc1c(F)c(F)c(F)c(F)c1F ; CNc1nc(N)nc2c1ncn2[C@@H]1C[C@](F)(CO[P@@](=O)(N[C@@H](C)C(=O)OC(C)C)Oc2ccccc2)[C@@H](O)[C@]1(F)Cl ; CNc1nc(N)nc2c1ncn2[C@@H]1C[C@](F)(CO[P@](=O)(N[C@@H](C)C(=O)OC(C)C)Oc2ccccc2)[C@@H](O)[C@]1(F)Cl]

Step 15: Synthesis of isopropyl (2S)-2-({[(2S,3R,4S,5R)-5-[2-amino-6-(methylamino)purin-9-yl]-4-chloro-2,4-difluoro-3-hydroxyoxolan-2-yl]methoxy (phenoxy)phosphoryl}amino)propanoate (58 & 59): To a stirred mixture of (2S,3R,4S,5R)-5-[2-amino-6-(methylamino)purin-9-yl]-4-chloro-2,4-difluoro-2-(hydroxymethyl)oxolan-3-ol (Compound 57, 50 mg, 0.143 mmol) and isopropyl (2S)-2-{[2,3,4,5,6-pentafluorophenoxy(phenoxy)phosphoryl]amino}propanoate (162 mg, 0.357 mmol) in THE (2 mL) and acetonitrile (0.2 mL) were added 1-methyl-1H-imidazole (29 mg, 0.357 mmol) dropwise at room temperature under nitrogen atmosphere. The resulting mixture was stirred at room temperature for additional 2 h. To the above mixture was added MgCl₂ (14 mg, 0.143 mmol) and DIEA (37 mg, 0.286 mmol) at room temperature. The resulting mixture was stirred at room temperature for additional 1 h. After completion of the reaction, the reaction was quenched with water. The crude product was purified by Prep-HPLC and SFC to give each isomer (Compound 58, 8.4 mg, the second peak in Prep-SFC and Compound 59, 7.0 mg, the first peak in Prep-SFC) as white solid. LCMS(ESI): m/z 619.2 [M+H]⁺.

Compound 62: ¹H NMR (400 MHz, DMSO-d6) δ 7.86 (s, 1H), 7.37 (t, J=7.9 Hz, 3H), 7.29-7.21 (m, 2H), 7.19 (t, J=7.3 Hz, 1H), 6.72 (d, J=15.0 Hz, 2H), 6.21-6.13 (m, 2H), 6.07 (dd, J=13.1, 10.0 Hz, 1H), 4.82 (m, 1H), 4.70 (brs, 1H), 4.33 (m, 1H), 3.85-3.73 (m, 1H), 2.88 (s, 3H), 1.19 (d, J=7.1 Hz, 3H), 1.12 (dd, J=6.3, 4.0 Hz, 6H). ¹⁹F NMR (376 MHz, DMSO-d6) δ −116.58.

Compound 63: ¹H NMR (400 MHz, DMSO-d6) δ 7.85 (s, 1H), 7.41-7.29 (m, 3H), 7.17 (d, J=7.6 Hz, 3H), 6.79 (s, 1H), 6.72 (d, J=14.9 Hz, 1H), 6.15 (s, 2H), 6.10-6.02 (m, 1H), 4.84 (m, 1H), 4.63 (s, 1H), 4.43 (m, 1H), 3.81 (m, 1H), 2.88 (s, 3H), 1.23 (d, J=7.1 Hz, 3H), 1.14 (d, J=6.2 Hz, 6H). ¹⁹F NMR (376 MHz, DMSO-d6) δ −116.28.

Procedure analogous to those for the synthesis of compound 58 and 59 were used for the synthesis of compound 61, 64, 65, 67 and 69 et al.

### Example 10

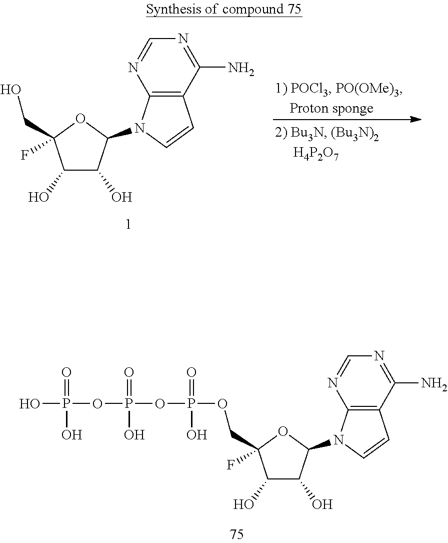 [SMILES: Nc1ncnc2c1ccn2[C@@H]1O[C@](F)(CO)[C@@H](O)[C@H]1O ; Nc1ncnc2c1ccn2[C@@H]1O[C@](F)(COP(=O)(O)OP(=O)(O)OP(=O)(O)O)[C@@H](O)[C@H]1O]

To a solution of 1 (130 mg, 457 mol), proton sponge (97.8 mg, 457 mol) in PO(OMe)₃ (1.30 mL) was added POCl₃ (104 mg, 685 mol, 63.5 μL) at −10° C. The mixture was stirred at 25° C. for 1 hr under N₂. After completion of the reaction, the crude product (182 mg, crude) was used into the next step without further purification.

To a solution of the crude product (182 mg, 347 μmol) in PO(OMe)₃ (1.30 mL) was added (Bu₃N)₂H4P₂O₇ (0.6 M, 2.30 mL) and Bu₃N (257 mg, 1388 μmol, 4.00 eq) in at −10° C. The mixture was stirred at 25° C. for 40 mins. After completion of the reaction, 1.00 M TEAB was added to adjust pH 7. The solution was diluted with H₂O and extracted with MTBE. The aqueous phase was concentrated by reduce pressure. The residue was purified by prep-HPLC to afford ((2S,3S,4R,5R)-5-(4-amino-7H-pyrrolo[2,3-d]pyrimidin-7-yl)-2-fluoro-3,4-dihydroxytetrahydrofuran-2-yl)methyl tetrahydrogen triphosphate (Compound 75, 13.0 mg, 24.0 mol, 10% yield) as a brown solid. ¹H NMR (400 MHz, D₂O) δ ppm 8.15 (s, 1H), 7.32 (d, J=3.60 Hz, 1H), 6.67 (d, J=4.0 Hz, 1H), 6.44 (d, J=2.0 Hz, 1H), 4.80-4.78 (m, 1H), 4.39-4.29 (i, 3H). ³¹P NMR (162 MHz, D₂O) δ ppm −10.4-−10.1 (1P), −11.9-−11.8 (HP), −22.9-−22.7 (1P) LCMS(ESI): 522.9 [M−H]⁻.

Procedure analogous to those for the synthesis of compound 75 were used for the synthesis of compound 76-85.

| Ex. # | Structure | Analytical data |
|---|---|---|
| 1 | 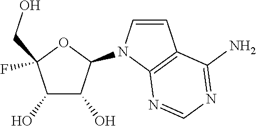 [SMILES: Nc1ncnc2c1ccn2[C@@H]1O[C@](F)(CO)[C@@H](O)[C@H]1O] | ¹H-NMR (400 MHz, DMSO-d₆): δ 8.07 (s, 1H), 7.26 (d, J = 3.6, 1H), 7.08 (s, 2H), 6.61 (d, J = 3.6 Hz, 1H), 6.33 (d, J = 3.2 Hz, 1H), 5.66 (d, J = 5.6 Hz, 1H), 5.38 (t, J = 6.4 Hz, 1H), 5.16 (d, J = 8.8 Hz, 1H), 4.52-4.44 (m, 1H), 4.38-4.34 (m, 1H), 3.54 (t, J = 6.4 Hz, 2H). LCMS(ESI): m/z 285.15 [M + H]⁺. |
| 2 | 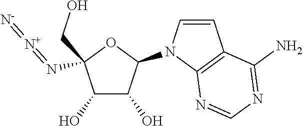 [SMILES: [N-]=[N+]=N[C@]1(CO)O[C@@H](n2ccc3c(N)ncnc32)[C@H](O)[C@@H]1O] | ¹H-NMR (400 MHz, DMSO-d₆ with D₂O @ HT): δ 8.06 (s, 1H), 7.29 (d, J = 3.6, 1H), 6.62 (d, J = 3.2 Hz, 1H), 6.25 (d, J = 6 Hz, 1H), 4.61 (t, J = 6.4 Hz, 1H), 4.33 (d, J = 5.6 Hz, 1H), 3.56 (d, J = 12 Hz, 1H), 3.46 (d, J = 11.6 Hz, 1H). LCMS(ESI): m/z 308.2 [M + H]⁺. |
| 3 | 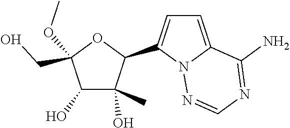 [SMILES: CO[C@]1(CO)O[C@@H](c2ccc3c(N)ncnn23)[C@](C)(O)[C@@H]1O] | ¹H-NMR (400 MHz, DMSO-d₆): δ 7.82 (s, 1H), 7.64 (s, 2H), 6.84 (d, J = 4.4 Hz, 1H), 6.72 (d, J = 4.4 Hz, 1H), 5.55 (s, 1H), 4.95 (d, J = 6.0 Hz, 1H), 4.49 (d, J = 9.2 Hz, 1H), 4.32 (s, 1H), 3.93 (d, J = 9.2 Hz, 1H), 3.69 (d, J = 5.6 Hz, 1H), 3.48 (d, J = 11.2 Hz, 1H), 3.27 (s, 3H), 0.75 (s, 3H). LCMS(ESI): m/z 311.1 [M + H]⁺. |
| 4 | 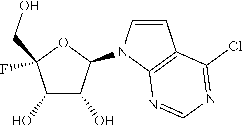 [SMILES: OC[C@@]1(F)O[C@@H](n2ccc3c(Cl)ncnc32)[C@H](O)[C@@H]1O] | ¹H-NMR (400 MHz, DMSO-d₆): δ 8.69 (d, J = 1.4 Hz, 1H), 7.87 (d, J = 3.7 Hz, 1H), 6.79 (d, J = 3.7 Hz, 1H), 6.47 (d, J = 3.0 Hz, 1H), 4.54 (dd, J = 16.4, 6.4 Hz, 1H), 4.44 (t, J = 4.8 Hz, 1H), 3.60-3.53 (m, 2H). LCMS(ESI): m/z 304.2 [M + H]⁺. |
| 5 | 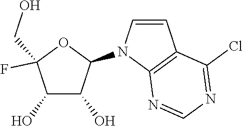 [SMILES: OC[C@]1(F)O[C@@H](n2ccc3c(Cl)ncnc32)[C@H](O)[C@@H]1O] | ¹H NMR (400 MHz, DMSO-d₆): δ 8.64 (s, 1H), 7.84-7.71 (m, 1H), 6.80 (d, J = 3.8 Hz, 1H), 6.44 (t, J = 7.1 Hz, 1H), 4.84 (dt, J = 7.3, 3.5 Hz, 1H), 4.16-4.05 (m, 1H), 3.73 (s, 1H), 3.52-3.40 (m, 1H). LCMS(ESI): m/z 304.2 [M + H]⁺. |
| 6 | 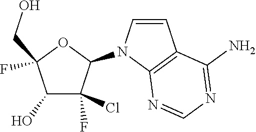 [SMILES: Nc1ncnc2c1ccn2[C@@H]1O[C@](F)(CO)[C@@H](O)[C@]1(F)Cl] | ¹H NMR (400 MHz, DMSO-d₆): δ 8.11 (s, 1H), 7.32-7.11 (m, 3H), 6.82 (d, J = 15.2 Hz, 2H), 6.69 (d, J = 3.8 Hz, 1H), 5.80 (s, 1H), 4.82 (t, J = 21.1 Hz, 1H), 3.72 (d, J = 5.6 Hz, 2H). LCMS(ESI): m/z 321.0 [M + H]⁺. |
| 7 | 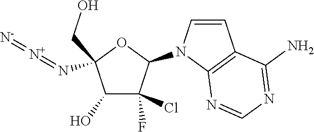 [SMILES: [N-]=[N+]=N[C@]1(CO)O[C@@H](n2ccc3c(N)ncnc32)[C@@](F)(Cl)[C@@H]1O] | ¹H NMR (400 MHz, DMSO-d₆): δ 8.10 (s, 1H), 7.34 (d, J = 3.6 Hz, 1H), 7.17 (s, 2H), 6.89 (d, J = 6.4 Hz, 1H), 6.78 (d, J = 16 Hz, 1H), 6.68 (d, J = 3.6 Hz, 1H), 5.89 (t, J = 5.6 Hz, 1H), 4.87 (d, J = 16.8 Hz, 1H), 3.84 (d, J = 5.6 Hz, 2H). LCMS(ESI): m/z 344.05 [M + H]⁺. |
| 8 | 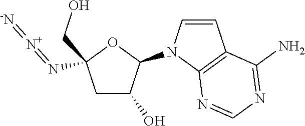 | ¹H-NMR (300 MHz, DMSO-d₆): δ 8.08 (s, 1H), 7.32 (d, J = 3.2 Hz, 1H), 7.07 (s, 2H), 6.62 (d, J = 3.6 Hz, 1H), 6.27 (d, J = 4.0 Hz, 1H), 5.73 (d, J = 4.8 Hz, 1H), 5.65 (t, J = 6.0 Hz, 1H), 4.65 (dd, J = 7.2, 4.4 Hz, 1H), 3.61-3.49 (m, 2H), 2.76-2.70 (m, 1H), 1.99-1.95 (m, 1H). LCMS(ESI): m/z 292.0 [M + H]⁺. |
| 9 | 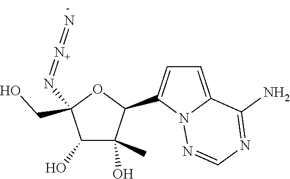 [SMILES: C[C@]1(O)[C@H](c2ccc3c(N)ncnn23)O[C@@](CO)(N=[N+]=N)[C@H]1O] | ¹H-NMR (300 MHz, DMSO-d₆): δ 7.85 (s, 1H), 7.71 (s, 2H), 6.86 (d, J = 4.5 Hz, 1H), 6.78 (d, J = 4.5 Hz, 1H), 5.71 (s, 1H), 5.46 (t, J = 6.0 Hz, 1H), 5.02 (d, J = 8.7 Hz, 1H), 4.97 (s, 1H), 4.02 (d, J = 8.8 Hz, 1H), 3.69-3.48 (m, 3H), 0.83 (s, 3H). LCMS(ESI): m/z 322.1 [M + H]⁺. |
| 10 | 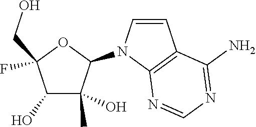 [SMILES: C[C@]1(O)[C@H](n2ccc3c(N)ncnc32)O[C@](F)(CO)[C@H]1O] | ¹H-NMR (300 MHz, DMSO-d₆): δ 8.06 (s, 1H), 7.34 (d, J = 3.8 Hz, 1H), 7.07 (s, 2H), 6.60 (d, J = 3.6 Hz, 1H), 6.45 (s, 1H), 5.65 (t, J = 5.8 Hz, 1H), 5.26 (d, J = 8.8 Hz, 1H), 5.14 (s, 1H), 4.17 (d, J = 20.0 Hz, 1H), 3.63 (d, J = 5.6 Hz, 2H), 0.69 (s, 3H). LCMS(ESI): m/z 299.15 [M + H]⁺. |
| 11 | 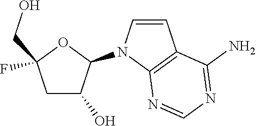 [SMILES: Nc1ncnc2c1ccn2[C@@H]1O[C@](F)(CO)C[C@H]1O] | ¹H-NMR (300 MHz, DMSO-d₆): δ 8.08 (s, 1H), 7.28 (d, J = 3.7 Hz, 1H), 7.09 (s, 2H), 6.62 (d, J = 3.7 Hz, 1H), 6.36 (d, J = 4.0 Hz, 1H), 5.74 (d, J = 4.8 Hz, 1H), 5.43 (t, J = 6.2 Hz, 1H), 4.61 (dd, J = 8.1, 4.1 Hz, 1H), 3.59-3.48 (m, 2H), 2.89 (m, 1H), 2.21-2.06 (m, 1H). ¹⁹F NMR (282 MHz, DMSO-d₆): δ −96.590. LCMS (ESI): m/z 269.1 [M + H]⁺. |
| 12 | 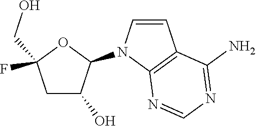 [SMILES: Nc1ncnc2c1ccn2[C@@H]1O[C@@](F)(CO)C[C@H]1O] | ¹H-NMR (400 MHz, DMSO-d₆): δ 8.08 (s, 1H), 7.24 (d, J = 4.0 Hz, 1H), 7.08 (s, 2H), 6.67 (d, J = 3.6 Hz, 1H), 6.29 (t, J = 6.0 Hz, 1H), 5.77 (d, J = 5.6 Hz, 1H), 5.36 (t, J = 6.4 Hz, 1H), 4.90-4.84 (m, 1H), 3.52-3.45 (m, 2H), 2.59-2.51 (m, 1H), 2.43-2.29 (m, 1H). ¹⁹F NMR (282 MHz, DMSO-d₆): δ −94.866. LCMS (ESI): m/z 269.1 [M + H]⁺. |
| 13 | 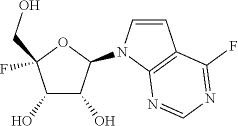 [SMILES: OC[C@@]1(F)O[C@@H](n2ccc3c(F)ncnc32)[C@H](O)[C@@H]1O] | ¹H-NMR (300 MHz, DMSO-d₆): δ 8.59 (d, J = 1.3 Hz, 1H), 7.87 (d, J = 3.8 Hz, 1H), 6.82 (d, J = 3.7 Hz, 1H), 6.49 (d, J = 3.0 Hz, 1H), 5.78 (d, J = 5.5 Hz, 1H), 5.38 (t, J = 6.1 Hz, 1H), 5.29 (d, J = 8.5 Hz, 1H), 4.54 (ddd, J = 16.3, 8.5, 6.3 Hz, 1H), 4.44 (td, J = 5.9, 3.1 Hz, 1H), 3.56 (t, J = 6.2 Hz, 2H). LCMS (ESI): m/z 288.1 [M + H]⁺ |
| 14 | 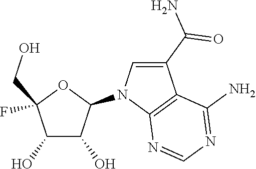 [SMILES: NC(=O)c1cn([C@@H]2O[C@](F)(CO)[C@@H](O)[C@H]2O)c2ncnc(N)c12] | ¹H-NMR (400 MHz, DMSO-d₆): δ 8.85 (s, 1H), 8.10 (d, J = 18.8 Hz, 2H), 7.90 (s, 1H), 7.51- 7.35 (m, 1H), 6.37 (d, J = 3.0 Hz, 1H), 5.80 (d, J = 5.6 Hz, 1H), 5.40-5.22 (m, 2H), 4.53 (dt, J = 16.4, 7.6 Hz, 1H), 4.37 (td, J = 6.0, 3.0 Hz, 1H), 3.57 (t, J = 6.4 Hz, 2H). ¹⁹FNMR (400 MHz, DMSO-d₆) δ −120.81. LCMS m/z [M + H]⁺ 328.1. |
| 15 | 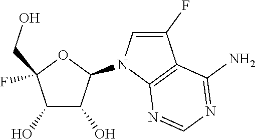 [SMILES: Nc1ncnc2c1c(F)cn2[C@@H]1O[C@](F)(CO)[C@@H](O)[C@H]1O] | ¹H NMR (400 MHz, MeOD-d4): δ 8.13 (s, 1H), 7.16 (d, J = 2.0 Hz, 1H), 6.48 (s, 1H), 4.62 (dd, J = 6.4, 7.4 Hz, 1H), 4.44 (dd, J = 6.4, 2.9 Hz, 1H), 3.84-3.72 (m, 2H). ¹⁹F NMR (377 MHz, MeOD-d4) δ −123.36 (s), −168.11 (s). LCMS (ESI): m/z 303.0 [M + H]⁺. |
| 16 | 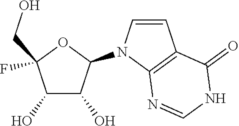 [SMILES: O=c1[nH]cnc2c1ccn2[C@@H]1O[C@](F)(CO)[C@@H](O)[C@H]1O] | LCMS (ESI): m/z 285.95 [M + H]⁺ |
| 17 | 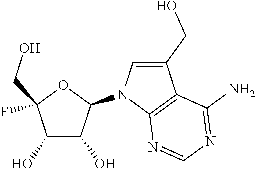 [SMILES: Nc1ncnc2c1c(CO)cn2[C@@H]1O[C@](F)(CO)[C@@H](O)[C@H]1O] | LCMS (ESI): m/z 315. [M + H]⁺ |
| 18 | 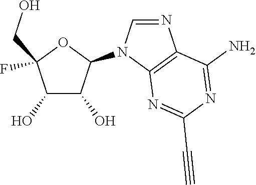 [SMILES: C#Cc1nc(N)c2ncn([C@@H]3O[C@](F)(CO)[C@@H](O)[C@H]3O)c2n1] | ¹H NMR (400 MHz, MeOD-d4): δ 8.37 (s, 1H), 6.29 (s, 1H), 4.74 (dd, J = 8 Hz, J = 16 Hz, 2H), 4.59 (m, 2H), 3.78 (d, J = 8 Hz, 3H), 3.55 (s, 1H). LCMS (ESI): m/z 310.3 [M + H]⁺. |
| 19 | 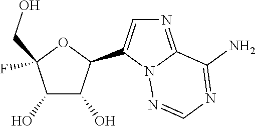 [SMILES: Nc1ncnn2c([C@@H]3O[C@](F)(CO)[C@@H](O)[C@H]3O)cnc12] | LCMS (ESI): m/z [M + H]⁺ 286.1 |
| 20 | 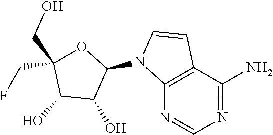 [SMILES: Nc1ncnc2c1ccn2[C@@H]1O[C@@](CO)(CF)[C@@H](O)[C@H]1O] | ¹H NMR (400 MHz, MeOD-d4) δ 8.06 (s, 1H), 7.32 (d, J = 3.1 Hz, 1H), 6.61 (d, J = 2.7 Hz, 1H), 5.98 (d, J = 7.7 Hz, 1H), 4.84-4.83 (m, 1H), 4.74 (d, J = 9.8 Hz, 1H), 4.60 (dd, J = 20.2, 9.8 Hz, 1H), 4.45 (d, J = 9.8 Hz, 1H), 4.32 (d, J = 5.1 Hz, 1H), 3.76 (s, 2H). ¹⁹F NMR (377 MHz, MeOD-d4) δ −236.74. LCMS(ESI): m/z 299.0 [M + H]⁺. |
| 21 | 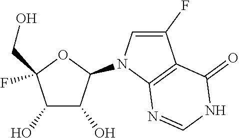 [SMILES: O=c1[nH]cnc2c1c(F)cn2[C@@H]1O[C@](F)(CO)[C@@H](O)[C@H]1O] | ¹H-NMR (400 MHz, DMSO-d₆): 11.88 (bs, 1H), 7.97 (s, 1H), 7.26 (s, 1H), 6.33 (s, 1H), 5.74 (s, 1H), 5.38 (bs, 1H), 5.23 (d, J = 5.2 Hz, 1H), 4.43-4.36 (m, 1H), 4.27 (s, 1H), 3.54 (s, 2H). LCMS (ESI): 304.15 [M + H]⁺ |
| 22 | 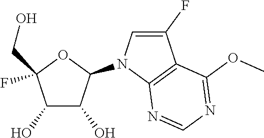 [SMILES: COc1ncnc2c1c(F)cn2[C@@H]1O[C@](F)(CO)[C@@H](O)[C@H]1O] | LCMS (ESI): 318.10 [M + H]⁺ |
| 23 | 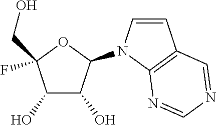 [SMILES: OC[C@@]1(F)O[C@@H](n2ccc3cncnc32)[C@H](O)[C@@H]1O] | LCMS (ESI): 270.15 [M + H]⁺ |
| 24 | 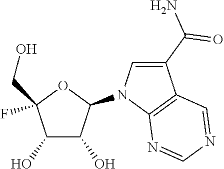 [SMILES: NC(=O)c1cn([C@@H]2O[C@](F)(CO)[C@@H](O)[C@H]2O)c2ncncc12] | ¹H-NMR (400 MHz, DMSO-d₆): δ 9.39 (s, 1H), 8.92 (s, 1H), 8.38 (s, 1H), 7.74 (s, 1H), 7.32 (s, 1H), 6.50 (d, J = 2.9 Hz, 1H), 5.86 (d, J = 5.4 Hz, 1H), 5.42-5.32 (m, 2H), 4.62-4.52 (m, 1H), 4.49-4.41 (m, 1H), 3.57 (t, J = 6.4 Hz, 2H). ¹⁹F NMR (377 MHz, MeOD-d4): δ −120.69. LCMS: m/z 313.1 [M + H]+. |
| 25 | 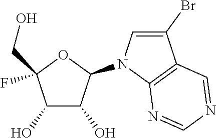 [SMILES: OC[C@@]1(F)O[C@@H](n2cc(Br)c3cncnc32)[C@H](O)[C@@H]1O] | 1H NMR (400 MHz, DMSO-d₆): δ 8.99 (s, 1H), 8.95 (s, 1H), 8.08 (s, 1H), 6.49 (d, J = 3.1 Hz, 1H), 6.05 (s, 1H), 5.40 (s, 1H), 4.58-4.50 (m, 1H), 4.48-4.43 (m, 1H), 3.57 (d, J = 6.3 Hz, 2H). ¹⁹F NMR (377 MHz, MeOD-d4) δ −120.79. LCMS (ESI): 348.0 [M + H]⁺ |
| 26 | 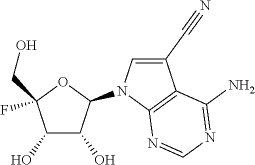 [SMILES: N#Cc1cn([C@@H]2O[C@](F)(CO)[C@@H](O)[C@H]2O)c2ncnc(N)c12] | ¹H NMR (400 MHz, DMSO-d₆): δ 8.37 (s, 1H), 8.26 (s, 1H), 6.96 (s, 2H), 6.37 (d, J = 2.8 Hz, 1H), 5.81 (d, J = 5.3 Hz, 1H), 5.45 (t, J = 6.0 Hz, 1H), 5.29 (d, J = 8.6 Hz, 1H), 4.51 (dt, J = 15.2, 7.1 Hz, 1H), 4.39 (s, 1H), 3.59 (t, J = 5.7 Hz, 2H). ¹⁹F NMR (376 MHz, DMSO-d₆): δ −120.55. LCMS (ESI): 310.1 [M + H]⁺ |
| 27 | 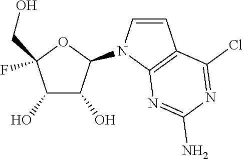 [SMILES: Nc1nc(Cl)c2ccn([C@@H]3O[C@](F)(CO)[C@@H](O)[C@H]3O)c2n1] | ¹H NMR (400 MHz, MeOD-d4): δ 7.19 (d, J = 4.0 Hz, 1H), 6.41 (d, J = 4.0 Hz, 1H), 6.32 (d, J = 2.4 Hz, 1H), 6.48 (s, 1H), 4.70 (dd, J= 17.2, 6.4, 1H), 4.44 (dd, J = 6.4, 2.4 Hz, 1H), 3.31-3.29 (m, 2H). ¹⁹F NMR (377 MHz, MeOD-d4): δ −124.56. LCMS (ESI): 319.1 [M + H]⁺ |
| 28 | 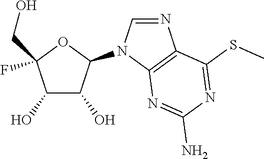 [SMILES: CSc1nc(N)nc2c1ncn2[C@@H]1O[C@](F)(CO)[C@@H](O)[C@H]1O] | ¹H NMR (400 MHz, DMSO-d₆)) δ 8.75 (d, J = 20.1 Hz, 1H), 8.66 (d, J = 10.8 Hz, 1H), 6.38-6.17 (m, 1H), 4.83-4.49 (m, 2H), 2.68 (d, J = 4.7 Hz, 4H). LCMS(ESI): m/z 332.3 [M + H]⁺ |
| 29 | 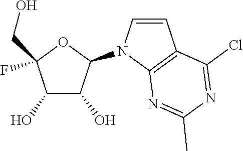 [SMILES: Cc1nc(Cl)c2ccn([C@@H]3O[C@](F)(CO)[C@@H](O)[C@H]3O)c2n1] | ¹H NMR (400 MHz, MeOD-d4): δ 7.63 (d, J = 3.8 Hz, 1H), 6.66 (d, J = 3.8 Hz, 1H), 6.51 (d, J = 2.7 Hz, 1H), 4.75 (dd, J = 16.6, 6.5 Hz, 1H), 4.51 (dd, J = 6.4, 2.8 Hz, 1H), 3.80-3.67 (m, 2H), 2.70 (s, 3H). LCMS(ESI): m/z 318.0 [M + H]⁺ |
| 30 | 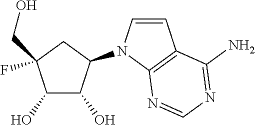 [SMILES: Nc1ncnc2c1ccn2[C@@H]1C[C@](F)(CO)[C@@H](O)[C@H]1O] | ¹H NMR (400 MHz, MeOD-d₄) δ 8.21 (s, 1H) 7.49 (d, J = 3.7 Hz, 1H), 6.85 (d, J = 3.6 Hz, 1H), 5.22 (dd, J = 17.0, 9.7 Hz, 1H), 4.47 (t, J = 6.7 Hz 1H), 4.21 (dd, J = 13.0, 6.2 Hz, 1H), 3.88-3.69 (m, 2H), 2.52 (ddd, J = 20.0, 14.8, 9.5 Hz, 1H), 2.42-2.25 (m, 1H). LCMS: m/z = 283.0 [M + H]⁺. |
| 31 | 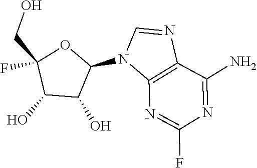 [SMILES: Nc1nc(F)nc2c1ncn2[C@@H]1O[C@](F)(CO)[C@@H](O)[C@H]1O] | H-NMR (400 MHz, DMSO-d₆): 8.31 (s, 1H), 7.9 (bs, 2H), 6.11 (s, 1H), 5.80 (d, J = 4 Hz, 1H), 5.36 (t, J = 4 Hz, 1H), 5.30 (d, J = 8 Hz, 1H), 4.52 (s, 2H), 3.56 (t, J = 8 Hz, 2H), 3.16 (d, J = 4 Hz, 1H). ¹⁹F NMR (282 MHz, DMSO-d₆) δ −51.65, −120.77. LCMS (ESI): m/z 304.3 [M + H]⁺ |
| 32 | 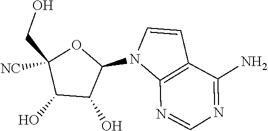 [SMILES: N#C[C@]1(CO)O[C@@H](n2ccc3c(N)ncnc32)[C@H](O)[C@@H]1O] | LCMS (ESI): m/z 292.15 [M + H]⁺ |
| 33 | 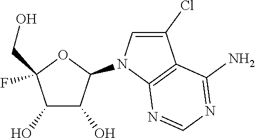 [SMILES: Nc1ncnc2c1c(Cl)cn2[C@@H]1O[C@](F)(CO)[C@@H](O)[C@H]1O] | ¹H NMR (400 MHz, MeOD-d4): δ 8.10 (s, 1H), 7.38 (s, 1H), 6.41 (d, J = 2.7 Hz, 1H), 4.65- 4.58 (m, 1H), 4.42-4.39 (m, 1H), 3.77-3.68 (m, 2H). ¹⁹F NMR (377 MHz, MeOD-d4): δ −124.16. LCMS (ESI): m/z 319.1 [M + H]⁺ |
| 34 | 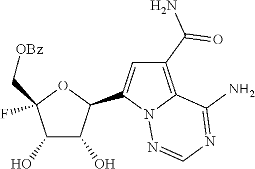 | LCMS (ESI): m/z 432.3 [M + H]⁺ |
| 35 | 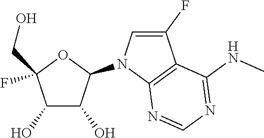 [SMILES: CNc1ncnc2c1c(F)cn2[C@@H]1O[C@](F)(CO)[C@@H](O)[C@H]1O] | ¹H-NMR (400 MHz, DMSO-d₆): 8.19 (s, 1H), 7.28 (d, J = 1.6 Hz, 1H), 6.38 (bs, 1H), 5.70 (d, J = 5.6 Hz, 1H), 5.36 (t, J = 6.0 Hz, 1H), 5.19 (d, J = 8.8 Hz, 1H), 4.65-4.55 (m, 1H), 4.47-4.39 (m, 1H), 4.31-4.27 (m, 1H), 3.54 (t, J = 6.0 Hz, 2H), 2.93 (d, J = 4.4 Hz, 3H). LCMS (ESI): m/z 317.1 [M + H]⁺ |
| 36 | 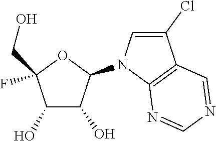 [SMILES: OC[C@@]1(F)O[C@@H](n2cc(Cl)c3cncnc32)[C@H](O)[C@@H]1O] | ¹H NMR (400 MHz, MeOD-d4): δ 8.99 (s, 1H), 8.88 (s, 1H), 7.82 (s, 1H), 6.59 (s, 1H), 4.71 (dd, J = 16.0 Hz, 8.0 Hz, 1 H), 4.50 (m, 1H), 3.75 (m, 2H). ¹⁹F NMR (377 MHz, MeOD-d4): δ −124.34. LCMS (ESI): m/z 304.1 [M + H]⁺ |
| 37 | 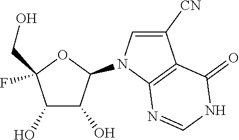 [SMILES: N#Cc1cn([C@@H]2O[C@](F)(CO)[C@@H](O)[C@H]2O)c2nc[nH]c(=O)c12] | ¹H NMR (400 MHz, MeOD-d4): δ 8.08 (s, 1H), 8.03 (s, 1H), 6.42 (d, J = 2.2 Hz, 1H), 4.64 (dd, J = 17.3, 6.3 Hz, 1H), 4.42 (dd, J = 6.3, 2.3 Hz, 1H), 3.84-3.68 (m, 2H). ¹⁹F NMR (377 MHz, MeOD-d4): δ −124.04 (dt, J = 8.9, 4.2 Hz). LCMS (ESI): m/z 311.1 [M + H]⁺ |
| 38 | 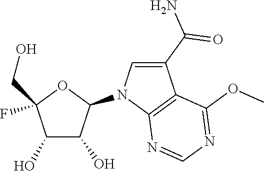 [SMILES: COc1ncnc2c1c(C(N)=O)cn2[C@@H]1O[C@](F)(CO)[C@@H](O)[C@H]1O] | ¹H NMR (400 MHz, MeOD-d4): δ 8.50 (s, 1H), 8.21 (s, 1H), 6.56 (d, J = 2.6 Hz, 1H), 4.72 (dd, J = 16.7, 6.4 Hz, 1H), 4.50 (dd, J = 6.4, 2.6 Hz, 1H), 4.22 (s, 3H), 3.81-3.71 (m, 2H). ¹⁹F NMR (377 MHz, MeOD-d4): δ −124.28 (s). LCMS (ESI): m/z 343.2 [M + H]⁺ |
| 39 | 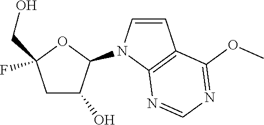 [SMILES: COc1ncnc2c1ccn2[C@@H]1O[C@](F)(CO)C[C@H]1O] | LCMS (ESI): m/z 284.3 [M + H]⁺ |
| 40 | 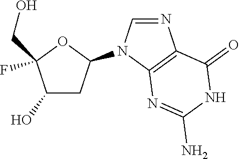 [SMILES: Nc1nc2c(ncn2[C@H]2C[C@H](O)[C@@](F)(CO)O2)c(=O)[nH]1] | ¹H NMR (400 MHz, DMSO-d₆): δ 8.46 (s, 1H), 7.55 (d, J = 3.7 Hz, 1H), 6.63 (d, J = 3.7 Hz, 1H), 6.43 (d, J = 3.2 Hz, 1H), 4.52 (dd, J = 16.4, 6.4 Hz, 1H), 4.41 (dd, J = 6.7, 3.2 Hz, 1H), 4.05 (s, 3H), 3.56 (d, J = 6.5 Hz, 2H). LCMS (ESI): m/z 300.2 [M + H]⁺ |
| 41 | 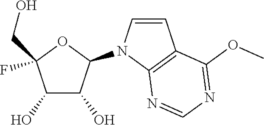 [SMILES: COc1ncnc2c1ccn2[C@@H]1O[C@](F)(CO)[C@@H](O)[C@H]1O] | ¹H NMR (400 MHz, DMSO-d₆): δ 8.46 (s, 1H), 7.55 (d, J = 3.7 Hz, 1H), 6.63 (d, J = 3.7 Hz, 1H), 6.43 (d, J = 3.2 Hz, 1H), 4.52 (dd, J = 16.4, 6.4 Hz, 1H), 4.41 (dd, J = 6.7, 3.2 Hz, 1H), 4.05 (s, 3H), 3.56 (d, J = 6.5 Hz, 2H). LCMS(ESI): m/z 300.2 [M + H]+ |
| 42 | 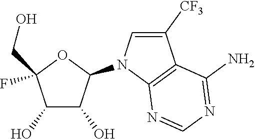 [SMILES: Nc1ncnc2c1c(C(F)(F)F)cn2[C@@H]1O[C@](F)(CO)[C@@H](O)[C@H]1O] | ¹H NMR (400 MHz, D2O) δ 8.01 (s, 1H), 7.68 (s, 1H), 6.36 (s, 1H), 4.61-4.54 (m, 1H), 4.47 (d, J = 5.8 Hz, 1H), 3.75 (d, J = 7.3 Hz, 2H). ¹⁹F NMR (377 MHz, D2O) δ −55.11, −55.85, −56.27, −122.08. LCMS(ESI): m/z 353.1 [M + H]+. |
| 43 | 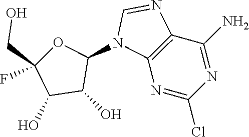 [SMILES: Nc1nc(Cl)nc2c1ncn2[C@@H]1O[C@](F)(CO)[C@@H](O)[C@H]1O] | ¹H NMR (400 MHz, DMSO-d₆): δ 8.35 (s, 2H), 6.14 (s, 1H), 5.81 (s, 1H), 5.35 (d, J = 20 Hz, 3 Hz), 4.51 (m, 4H) LCMS (ESI): m/z 320.2 [M + H]⁺ |
| 44 | 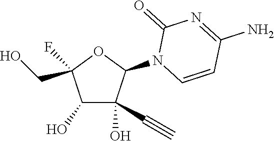 [SMILES: C#C[C@]1(O)[C@H](n2ccc(N)nc2=O)O[C@](F)(CO)[C@H]1O] | LCMS (ESI): m/z 286.3 [M + H]⁺ |
| 45 | 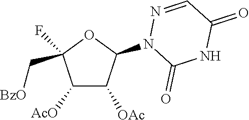 | ¹H NMR (400 MHz, MeOD-d4): δ 8.05- 7.96 (m, 2H), 7.65 (t, J = 7.5 Hz, 1H), 7.51 (t, J = 7.8 Hz, 2H), 7.19 (s, 1H), 6.49 (s, 1H), 5.83 (dd, J = 19.3, 6.4 Hz, 1H), 5.62 (d, J = 6.4 Hz, 1H), 4.69 (dd, J = 12.1, 9.0 Hz, 1H), 4.48 (dd, J = 12.1, 4.9 Hz, 1H), 2.14 (s, 3H), 2.05 (s, 3H). ¹⁹F NMR (377 MHz, MeOD-d4): δ −117.39 (s). LCMS (ESI): m/z 925.1 [2M + Na]⁺ |
| 46 | 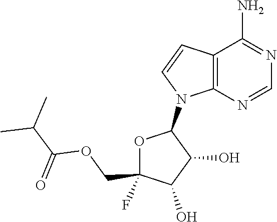 [SMILES: CC(C)C(=O)OC[C@@]1(F)O[C@@H](n2ccc3c(N)ncnc32)[C@H](O)[C@@H]1O] | ¹H NMR (300 MHz, DMSO-d₆): δ 8.09 (s, 1H), 7.30 (d, J = 3.6 Hz, 1H), 7.14 (s, 2H), 6.64 (d, J = 3.6 Hz, 1H), 6.31 (d, J = 2.2 Hz, 1H), 5.81 (s, 1H), 5.34 (s, 1H), 4.66 (d, J = 19.1 Hz, 1H), 4.53 (s, 1H), 4.33 (dd, J = 11.9, 7.9 Hz, 1H), 4.19- 4.10 (m, 1H), 2.50-2.43 (m, 1H), 1.05 (d, J = 7.0 Hz, 3H), 1.00 (d, J = 7.0 Hz, 3H). LCMS (ESI): m/z 355.1 [M + H]⁺ |
| 47 | 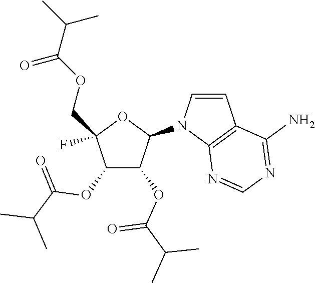 [SMILES: CC(C)C(=O)OC[C@@]1(F)O[C@@H](n2ccc3c(N)ncnc32)[C@H](OC(=O)C(C)C)[C@@H]1OC(=O)C(C)C] | ¹H NMR (300 MHz, DMSO-d₆): δ 8.09 (s, 1H), 7.37 (d, J = 3.6 Hz, 1H), 7.17 (s, 2H), 6.64 (d, J = 3.6 Hz, 1H), 6.50 (d, J = 1.8 Hz, 1H), 6.25 (dd, J = 19.1, 7.0 Hz, 1H), 5.93 (dd, J = 7.0, 2.1 Hz, 1H), 4.42-4.23 (m, 2H), 2.63 (dt, J = 13.9, 6.9 Hz, 2H), 2.48-2.40 (m, 1H), 1.15 (d, J = 6.9 Hz, 6H), 1.13-1.02 (m, 9H), 0.97 (d, J = 7.0 Hz, 3H). LCMS (ESI): m/z [M + H]⁺ 495.3 |
| 48 | 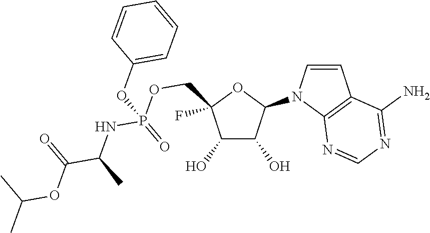 [SMILES: CC(C)OC(=O)[C@H](C)N[P@](=O)(OC[C@@]1(F)O[C@@H](n2ccc3c(N)ncnc32)[C@H](O)[C@@H]1O)Oc1ccccc1] | ¹H NMR (300 MHz, DMSO-d₆): δ 8.08 (s, 1H), 7.32 (t, J = 7.7 Hz, 2H), 7.24 (d, J = 3.6 Hz, 1H), 7.15 (m, 5H), 6.62 (d, J = 3.7 Hz, 1H), 6.35 (d, J = 3.2 Hz, 1H), 6.06 (dd, J = 13.1, 10.1 Hz, 1H), 5.81 (d, J = 5.6 Hz, 1H), 5.40 (d, J = 8.5 Hz, 1H), 4.82 (m, 1H), 4.58 (m, 1H), 4.46 (m, 1H), 4.14 (m, 2H), 3.83-3.65 (m, 1H), 1.22-0.98 (m, 9H). ¹⁹F NMR (282 MHz, DMSO-d₆): δ −120.63. LCMS (ESI): m/z 554.1 [M + H]⁺ |
| 49 | 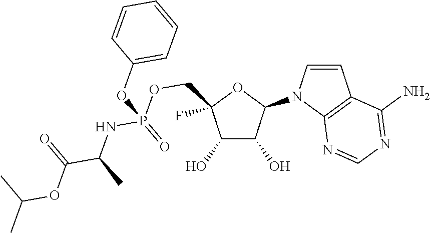 [SMILES: CC(C)OC(=O)[C@H](C)N[P@@](=O)(OC[C@@]1(F)O[C@@H](n2ccc3c(N)ncnc32)[C@H](O)[C@@H]1O)Oc1ccccc1] | ¹H NMR (300 MHz, DMSO-d₆): δ 8.08 (s, 1H), 7.34 (t, J = 7.8 Hz, 2H), 7.24-7.08 (m, 6H), 6.63 (d, J = 3.7 Hz, 1H), 6.36 (d, J = 3.0 Hz, 1H), 6.03 (dd, J = 13.4, 9.9 Hz, 1H), 5.82 (d, J = 5.4 Hz, 1H), 5.43 (d, J = 8.7 Hz, 1H), 4.83 (m, 1H), 4.58 (m, 1H), 4.46 (s, 1H), 4.17 (m, 2H), 3.69 (m, 1H), 1.13 (d, J = 5.8 Hz, 9H). ¹⁹F NMR (282 MHz, DMSO-d₆): δ −120.27. LCMS (ESI): m/z 554.1 [M + H]⁺ |
| 50 | 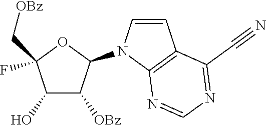 | ¹H NMR (400 MHz, DMSO-d₆): 8.74 (s, 1H), 8.15 (d, J = 8 Hz, 2H), 7.93 (d, J = 4 Hz, 2H), 7.82 (J = 8 Hz, 2H), 7.64 (m, 1H), 7.52 (t, J = 8 Hz, 4H), 7.41 (m, 3H), 6.80 (d, J = 4 Hz, 2H), 6.19 (d, J = 4 Hz, 1H), 5.59 (dd, J = 4 Hz, J = 20 Hz), 4.65 (m, 2H). LCMS (ESI): m/z 503.4 [M + H]⁺ |
| 51 | 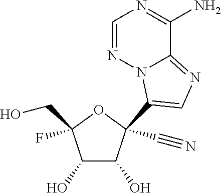 [SMILES: N#C[C@@]1(c2cnc3c(N)ncnn23)O[C@](F)(CO)[C@@H](O)[C@H]1O] | ¹H NMR (400 MHz, DMSO-d₆): 8.13 (s, 1H), 7.84 (s, 1H), 4.76 (d, J = 4 Hz, 1H), 4.6 (s, 2H), 4.49 (dd, J = 4 Hz, J = 20 Hz), 3.77-3.79 (m, 2H). LCMS (ESI): m/z 311.2 [M + H]⁺ |
| 52 | 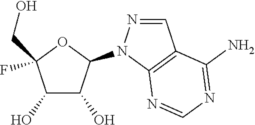 [SMILES: Nc1ncnc2c1cnn2[C@@H]1O[C@](F)(CO)[C@@H](O)[C@H]1O] | LCMS (ESI): m/z 286.0 [M + H]⁺. |
| 53 | 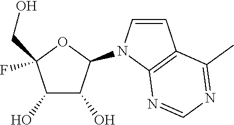 [SMILES: Cc1ncnc2c1ccn2[C@@H]1O[C@](F)(CO)[C@@H](O)[C@H]1O] | ¹H-NMR (400 MHz, MeOD-d4 with D₂O): δ 8.67 (s, 1H), 7.62-7.61 (d, J = 3.2, 1H), 6.83-6.82 (d, J = 3.2 Hz, 1H), 6.56-6.55 (d, J = 2.4 Hz, 1H), 4.58-4.56 (m, 2H), 3.80-3.77 (m, 2H), 2.74 (s, 1H). LCMS (ESI): m/z 284.0 [M + H]⁺ |
| 54 | 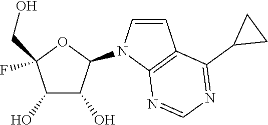 [SMILES: OC[C@@]1(F)O[C@@H](n2ccc3c(C4CC4)ncnc32)[C@H](O)[C@@H]1O] | 1H NMR (400 MHz, MeOD-d4): δ 8.55 (s, 1H), 7.57 (d, J = 3.8 Hz, 1H), 6.83 (d, J = 3.8 Hz, 1H), 6.53 (d, J = 2.7 Hz, 1H), 4.73 (dd, J = 16.6, 6.5 Hz, 1H), 4.52 (dd, J = 6.4, 2.8 Hz, 1H), 3.81- 3.67 (m, 2H), 2.51-2.42 (m, 1H), 1.32-1.25 (m, 2H), 1.23-1.15 (m, 2H). LCMS (ESI): m/z 310.0 [M + H]⁺ |
| 55 | 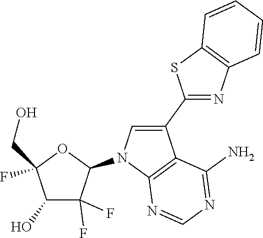 [SMILES: Nc1ncnc2c1c(-c1nc3ccccc3s1)cn2[C@@H]1O[C@](F)(CO)[C@@H](O)C1(F)F] | ¹H NMR (400 MHz, DMSO-d₆): δ 9.26 (s, 1H), 8.35 (s, 1H), 8.22 (s, 1H), 8.13 (d, J = 7.7 Hz, 1H), 8.00 (d, J = 8.0 Hz, 1H), 7.79 (s, 1H), 7.51 (dt, J = 32.0, 7.2 Hz, 2H), 6.77 (d, J = 10.7 Hz, 2H), 5.92 (s, 1H), 5.01 (s, 1H), 3.79 (t, J = 5.8 Hz, 2H). ¹⁹F NMR (376 MHz, DMSO-d₆): δ −116.50, −117.13, −124.69. LCMS (ESI): m/z 438.1 [M + H]⁺ |
| 56 | 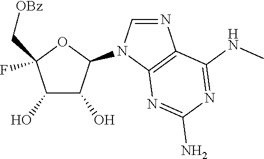 | ¹H NMR (400 MHz, MeOD-d4): δ 7.96 (d, J = 8.4 Hz, 2H), 7.58 (t, J = 7.2 Hz, 1H), 7.45 (t, J = 8.4 Hz, 2H), 6.72 (d, J = 4.0 Hz, 1H), 6.33 (d, J = 4.0 Hz, 1H), 6.22 (d, J = 2.0 Hz, 1H), 4.99-4.96 (m, 1H), 4.66-4.47 (m, 3H), 2.96 (s, 3H). ¹⁹F NMR (377 MHz, MeOD-d4): δ −121.34. LCMS (ESI): m/z 418.1 [M + H]⁺ |
| 57 | 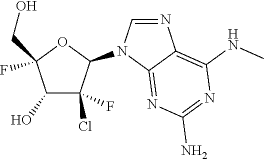 [SMILES: CNc1nc(N)nc2c1ncn2[C@@H]1O[C@](F)(CO)[C@@H](O)[C@]1(F)Cl] | LCMS m/z [M + H]⁺ 351.0 |
| 58 | 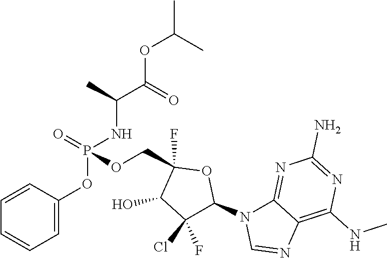 [SMILES: CNc1nc(N)nc2c1ncn2[C@@H]1O[C@](F)(CO[P@@](=O)(N[C@@H](C)C(=O)OC(C)C)Oc2ccccc2)[C@@H](O)[C@]1(F)Cl] | ¹H NMR (400 MHz, DMSO-d₆): δ 7.86 (s, 1H), 7.37 (t, J = 7.9 Hz, 3H), 7.29-7.21 (m, 2H), 7.19 (t, J = 7.3 Hz, 1H), 6.72 (d, J = 15.0 Hz, 2H), 6.21-6.13 (m, 2H), 6.07 (dd, J = 13.1, 10.0 Hz, 1H), 4.82 (m, 1H), 4.70 (brs, 1H), 4.33 (m, 1H), 3.85-3.73 (m, 1H), 2.88 (s, 3H), 1.19 (d, J = 7.1 Hz, 3H), 1.12 (dd, J = 6.3, 4.0 Hz, 6H). ¹⁹F NMR (376 MHz, DMSO-d₆):) δ −116.58. LCMS (ESI): m/z 619.2 [M + H]⁺ |
| 59 | 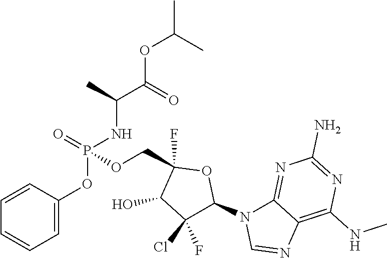 [SMILES: CNc1nc(N)nc2c1ncn2[C@@H]1O[C@](F)(CO[P@](=O)(N[C@@H](C)C(=O)OC(C)C)Oc2ccccc2)[C@@H](O)[C@]1(F)Cl] | ¹H NMR (400 MHz, DMSO-d₆): δ 7.85 (s, 1H), 7.41-7.29 (m, 3H), 7.17 (d, J = 7.6 Hz, 3H), 6.79 (s, 1H), 6.72 (d, J = 14.9 Hz, 1H), 6.15 (s, 2H), 6.10-6.02 (m, 1H), 4.84 (m, 1H), 4.63 (s, 1H), 4.43 (m, 1H), 3.81 (m, 1H), 2.88 (s, 3H), 1.23 (d, J = 7.1 Hz, 3H), 1.14 (d, J = 6.2 Hz, 6H). ¹⁹F NMR (376 MHz, DMSO-d₆): δ −116.28. LCMS (ESI): m/z 619.2 [M + H]⁺ |
| 60 | 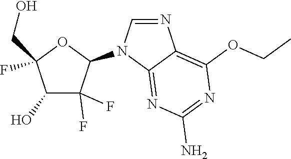 [SMILES: CCOc1nc(N)nc2c1ncn2[C@@H]1O[C@](F)(CO)[C@@H](O)C1(F)F] | LCMS (ESI): m/z 350.1 [M + H]⁺ |
| 61 | 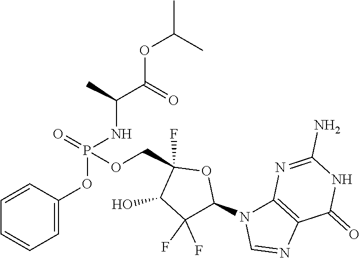 [SMILES: CC(C)OC(=O)[C@H](C)NP(=O)(OC[C@@]1(F)O[C@@H](n2cnc3c(=O)[nH]c(N)nc32)C(F)(F)[C@@H]1O)Oc1ccccc1] | ¹H NMR (400 MHz, MeOD-d4): δ 8.33 (d, J = 17.1 Hz, 1H), 7.41-7.17 (m, 5H), 6.84- 6.72 (m, 1H), 5.90 (s, 1H), 5.04-4.93 (m, 2H), 4.03-3.92 (m, 2H), 3.86-3.79 (m, 1H), 1.40- 1.32 (m, 3H), 1.25-1.19 (m, 6H). ¹⁹F NMR (377 MHz, MeOD-d4): δ −111.23, −115.82, −116.46, −126.63, −126.99. ³¹P NMR (162 MHz, MeOD-d4): δ 3.55, 3.33. LCMS (ESI): m/z 591.1 [M + H]⁺ |
| 62 | 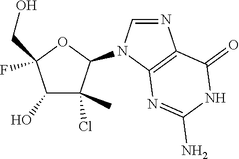 [SMILES: C[C@]1(Cl)[C@H](n2cnc3c(=O)[nH]c(N)nc32)O[C@](F)(CO)[C@H]1O] | ¹H NMR (400 MHz, MeOD-d4): δ 8.15 (s, 1H), 6.65 (s, 1H), 4.63 (d, J = 18.4 Hz, 1H), 3.84-3.81 (m, 2H), 1.36 (s, 3H). ¹⁹F NMR (377 MHz, MeOD-d4): δ −123.103. LCMS (ESI): m/z 334.0 [M + H]⁺ |
| 63 | 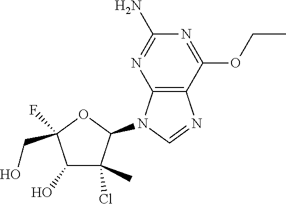 [SMILES: CCOc1nc(N)nc2c1ncn2[C@@H]1O[C@](F)(CO)[C@@H](O)[C@@]1(C)Cl] | ¹H NMR (400 MHz, MeOD-d4): δ 8.23 (s, 1H), 6.71 (s, 1H), 4.58-4.50 (m, 3H), 3.87- 3.83 (m, 2H), 1.43 (t, J = 7.1 Hz, 3H), 1.35 (s, 3H). ¹⁹F NMR (377 MHz, MeOD-d4): δ −123.406. LCMS (ESI): m/z 362.1 [M + H]⁺ |
| 64 | 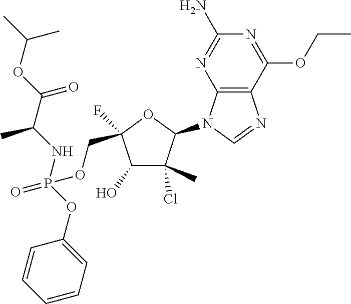 [SMILES: CCOc1nc(N)nc2c1ncn2[C@@H]1O[C@](F)(COP(=O)(N[C@@H](C)C(=O)OC(C)C)Oc2ccccc2)[C@@H](O)[C@@]1(C)Cl] | ¹H NMR (400 MHz, MeOD-d4): δ 7.95- 7.92 (m, 1H), 7.35-7.15 (m, 5H), 7.71-7.69 (m, 1H), 5.30-5.25 (m, 2H), 4.97-4.88 (m, 2H), 4.72-4.64 (m, 1H), 4.52-4.40 (m, 3H), 3.94-3.87 (m, 1H), 1.44-1.17 (m, 15H). ¹⁹F NMR (377 MHz, MeOD-d4): δ −121.736. ³¹P NMR (162 MHz, MeOD-d4): δ 4.217, 4.120. LCMS (ESI): m/z 631.0 [M + H]⁺ |
| 65 | 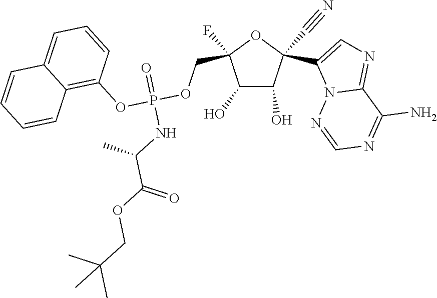 [SMILES: C[C@H](NP(=O)(OC[C@@]1(F)O[C@@](C#N)(c2cnc3c(N)ncnn23)[C@H](O)[C@@H]1O)Oc1cccc2ccccc12)C(=O)OCC(C)(C)C] | ¹H NMR (400 MHz, MeOD-d4): δ 8.29- 8.16 (m, 1H), 8.10-8.01 (m, 1H), 7.94-7.83 (m, 2H), 7.76-7.68 (m, 1H), 7.61-7.39 (m, 4H), 5.79-5.55 (m, 1H), 5.31-5.15 (m, 1H), 4.17-4.06 (m, 1H), 3.95-3.90 (m, 1H), 3.86- 3.76 (m, 2H), 3.76-3.65 (m, 1H), 1.42-1.35 (m, 3H), 0.95-0.83 (m, 9H). ¹⁹F NMR (377 MHz, MeOD-d4): δ −122.86, −122.98. ³¹P NMR (162 MHz, MeOD-d4): δ 3.61, 3.36. LCMS (ESI): m/z 658.1 [M + H]⁺ |
| 66 | 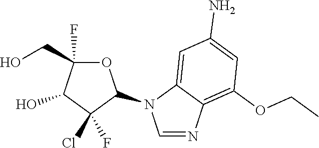 | ¹H NMR (400 MHz, MeOD-d4): δ = 8.05 (s, 1H), 6.66 (d, J = 14.0 Hz, 1H), 5.08 (br t, J = 20.0 Hz, 1H), 4.55 (q, J = 7.2 Hz, 2H), 3.88 (d, J = 5.6 Hz, 2H), 1.44 (t, J = 7.2 Hz, 3H). ¹⁹F NMR (377 MHz, MeOD-d4): δ = −118.62- −119.68 (m, 1F), −126.56 (br d, J = 15.7 Hz, 1F). LCMS (ESI): m/z 366.2 [M + H]⁺ |
| 67 | 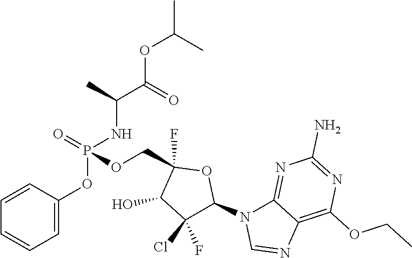 [SMILES: CCOc1nc(N)nc2c1ncn2[C@@H]1O[C@](F)(CO[P@@](=O)(N[C@@H](C)C(=O)OC(C)C)Oc2ccccc2)[C@@H](O)[C@]1(F)Cl] | ¹H NMR (400 MHz, MeOD-d4): δ = 7.93 (d, J = 11.6 Hz, 1H), 7.37-7.28 (m, 2H), 7.25 (br d, J = 7.6 Hz, 1H), 7.21-7.13 (m, 2H), 6.68 (dd, J = 3.2, 14.4 Hz, 1H), 5.69-5.37 (m, 1H), 4.97-4.94 (m, 1H), 4.76-4.66 (m, 1H), 4.53 (q, J = 7.2 Hz, 2H), 4.50-4.39 (m, 1H), 4.00- 3.82 (m, 1H), 1.44 (t, J = 7.2 Hz, 3H), 1.34- 1.28 (m, 3H), 1.24-1.16 (m, 6H). ¹⁹F NMR (377 MHz, MeOD-d4): δ = −118.81 (br s, 1F), −118.85 (br s, 1F). ³¹P NMR (162 MHz, MeOD- d4): δ = 4.31-3.95 (m, 1P). LCMS (ESI): m/z 635.2 [M + H]⁺ |
| 68 | 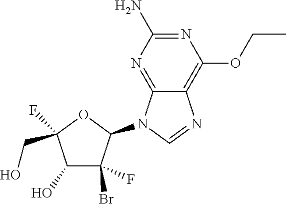 [SMILES: CCOc1nc(N)nc2c1ncn2[C@@H]1O[C@](F)(CO)[C@@H](O)[C@]1(F)Br] | ¹H NMR (400 MHz, MeOD-d4): δ = 8.06 (s, 1H), 6.69 (d, J = 14.8 Hz, 1H), 5.20-5.11 (m, 1H), 4.55 (q, J = 7.2 Hz, 2H), 3.89 (d, J = 6.0 Hz, 2H), 1.44 (t, J = 7.2 Hz, 3H). ¹⁹F NMR (400 MHz, MeOD-d4): δ = −117.80, −126.04. LCMS (ESI): m/z 410.1 [M + H]⁺ |
| 69 | 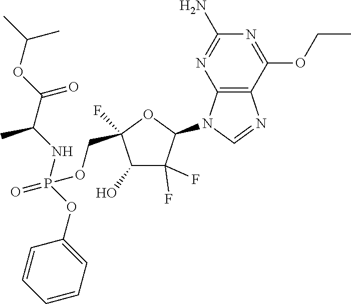 [SMILES: CCOc1nc(N)nc2c1ncn2[C@@H]1O[C@](F)(COP(=O)(N[C@@H](C)C(=O)OC(C)C)Oc2ccccc2)[C@@H](O)C1(F)F] | ¹H NMR (400 MHz, MeOD-d4): δ 8.46 (d, J = 13.9 Hz, 1H), 7.42-7.36 (m, 2H), 7.28- 7.21 (m, 3H), 6.74 (d, J = 11.1 Hz, 1H), 5.74- 5.61 (m, 1H), 4.99-4.92 (m, 1H), 4.63-4.54 (m, 2H), 3.99-3.89 (m, 1H), 3.82-3.74 (m, 2H), 1.45 (t, J = 7.1 Hz, 3H), 1.32 (dd, J = 7.2, 1.0 Hz, 3H), 1.21-1.18 (m, 6H). ¹⁹F NMR (377 MHz, MeOD-d4): δ −113.10, −117.39, −117.66, −118.05, −123.63, −126.23. ³¹P NMR (162 MHz, MeOD-d4): δ 3.82, 3.21. LCMS (ESI): m/z 619.3 [M + H]⁺ |
| 70 | 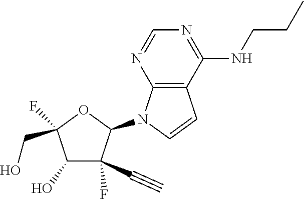 [SMILES: C#C[C@]1(F)[C@H](n2ccc3c(NCCC)ncnc32)O[C@](F)(CO)[C@H]1O] | ¹H NMR (400 MHz, MeOD-d4): δ 8.13 (s, 1H), 7.37 (d, J = 3.7 Hz, 1H), 6.62 (d, J = 3.7 Hz, 1H), 6.58 (s, 1H), 4.67 (t, J = 24.3 Hz, 1H), 3.81-3.74 (m, 2H), 3.45 (dd, J = 20.8, 13.5 Hz, 2H), 2.56 (s, 1H), 1.78-1.62 (m, 2H), 1.01 (t, J = 7.4 Hz, 3H). ¹⁹F NMR (377 MHz, MeOD-d4): δ −123.70 (s). LCMS (ESI): m/z 351.1 [M + H]⁺ |
| 71 | 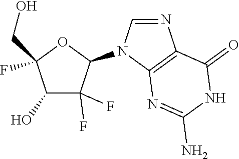 [SMILES: Nc1nc2c(ncn2[C@@H]2O[C@](F)(CO)[C@@H](O)C2(F)F)c(=O)[nH]1] | ¹H NMR (400 MHz, MeOD-d4): δ 8.32 (s, 1H), 6.74 (d, J = 11.5 Hz, 1H), 5.05 (s, 1H), 3.84 (d, J = 4.9 Hz, 2H). ¹⁹F NMR (377 MHz, MeOD-d4): δ −114.38, −119.14, −119.78, −128.40. LCMS (ESI): m/z 322.0 [M + H]⁺ |
| 72 | 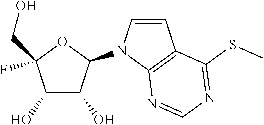 [SMILES: CSc1ncnc2c1ccn2[C@@H]1O[C@](F)(CO)[C@@H](O)[C@H]1O] | ¹H NMR (400 MHz, DMSO-d₆): δ 8.67 (d, J = 1.2 Hz, 1H), 7.68 (d, J = 3.6 Hz, 1H), 6.65 (d, J = 3.5 Hz, 1H), 6.45 (d, J = 3.0 Hz, 1H), 4.53 (dd, J = 16.8, 6.5 Hz, 1H), 4.46-4.39 (m, 1H), 2.67 (t, J = 3.0 Hz, 5H). LCMS(ESI): m/z 316.1 [M + H]+ |
| 73 | 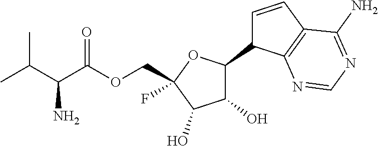 [SMILES: CC(C)[C@H](N)C(=O)OC[C@@]1(F)O[C@@H](C2C=Cc3c(N)ncnc32)[C@H](O)[C@@H]1O] | ¹H NMR (400 MHz, DMSO-d₆): δ 8.19 (s, 1H), 8.08 (s, 1H), 7.31 (d, J = 3.6 Hz, 1H), 7.10 (s, 2H), 6.36 (d, J = 3.6 Hz, 1H), 6.31 (d, J = 2.6 Hz, 1H), 4.67 (dd, J = 18.4, 6.8 Hz, 1H), 4.53 (dd, J = 6.8, 2.8 Hz, 1H), 4.36-4.20 (m, 2H), 3.14 (d, J = 5.2 Hz, 1H), 1.85 (dq, J = 13.2, 6.8 Hz, 1H), 0.81(dd, J = 16.4, 6.8 Hz, 6H). ¹⁹FNMR(400 MHz, DMSO-d₆): δ −74.43, −119.36. LCMS (ESI): m/z 384.2 [M + H]⁺ |
| 74 | 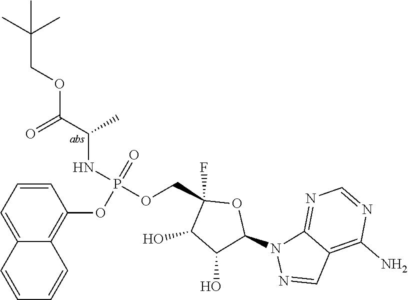 [SMILES: C[C@H](NP(=O)(OC[C@@]1(F)O[C@@H](n2ncc3c(N)ncnc32)[C@H](O)[C@@H]1O)Oc1cccc2ccccc12)C(=O)OCC(C)(C)C] | LCMS (ESI): m/z 633.0 [M + H]⁺ |
| 75 | 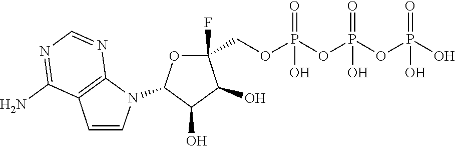 [SMILES: Nc1ncnc2c1ccn2[C@@H]1O[C@](F)(COP(=O)(O)OP(=O)(O)OP(=O)(O)O)[C@@H](O)[C@H]1O] | ¹H NMR (400 MHz, D₂O): δ 8.15 (s, 1H), 7.32 (d, J = 3.60 Hz, 1H), 6.67 (d, J = 4.0 Hz, 1H), 6.44 (d, J = 2.0 Hz, 1H), 4.80-4.78 (m, 1H), 4.39-4.29 (m, 3H). ³¹P NMR (162 MHz, D2O) δ −10.4-−10.1 (1P), −11.9-−11.8 (1P), −22.9-−22.7 (1P). LCMS(ESI): 522.9 [M − H]⁻. |
| 76 | 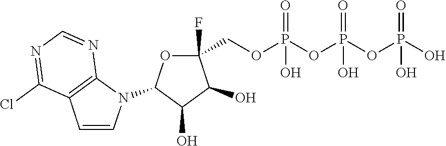 [SMILES: O=P(O)(O)OP(=O)(O)OP(=O)(O)OC[C@@]1(F)O[C@@H](n2ccc3c(Cl)ncnc32)[C@H](O)[C@@H]1O] | ¹H NMR ( 400 MHz, D₂O): δ 8.43 (s, 1H), 7.61 (d, J = 3.6 Hz, 1H), 6.68 (d, J = 4.0 Hz, 1H), 6.52 (d, J = 2.4 Hz, 1H), 4.80-4.76 (m, 1H), 4.20-4.18 (m, 1H), 4.19-4.05 (m, 1H). ³¹P NMR (162 MHz, D₂O): δ −10.8-−10.6 (1P), −12.2-12.1 (1P), −23.3-−23.0 (1P). ¹⁹F NMR (376 MHz, D₂O): δ −120.6 (1F). LCMS(ESI): 541.9 [M − H]⁻. |
| 77 | 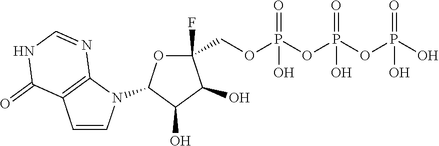 [SMILES: O=c1[nH]cnc2c1ccn2[C@@H]1O[C@](F)(COP(=O)(O)OP(=O)(O)OP(=O)(O)O)[C@@H](O)[C@H]1O] | ¹H NMR(400 MHz, D₂O): δ 8.04 (s, 1H), 7.33 (d, J = 2.0 Hz, 1H), 6.71 (d, J = 2.0 Hz, 1H), 6.50 (s, 1H), 4.89-4.83 (m, 1H), 4.58 (s, 1H), 4.28 (s, 1H), 4.14 (s, 1H). ³¹P NMR (162 MHz, D₂O): δ −9.93-−9.84 (1P), −12.0-−11.9 (1P), −23.0-−22.7 (1P). ¹⁹P NMR (376 MHz, D₂O): δ −120.5 (1F). LCMS(ESI): 523.9 [M − H]⁻. |
| 78 | 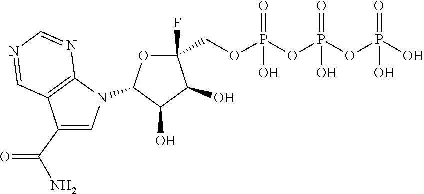 [SMILES: NC(=O)c1cn([C@@H]2O[C@](F)(COP(=O)(O)OP(=O)(O)OP(=O)(O)O)[C@@H](O)[C@H]2O)c2ncncc12] | ¹H NMR (400 MHz, D₂O): δ 9.29 (s, 1H), 8.85 (s, 1H), 8.48 (s, 1H), 6.73 (s, 1H), 4.98- 4.77 (m, 1H), 4.53 (d, J = 6.4 Hz, 1H), 4.32-4.34 (m, 1H), 4.17-4.20 (m, 1H). ³¹P NMR (162 MHz, D₂O): δ −9.49-−9.40 (1P), −12.1-−11.9 (1P), −22.9- −22.5 (1P). ¹⁹F NMR (376 MHz, D₂O): δ −120.0 (1F). LCMS(ESI): 551.0 [M − H]⁻. |
| 79 | 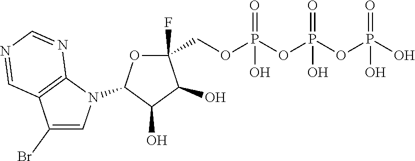 [SMILES: O=P(O)(O)OP(=O)(O)OP(=O)(O)OC[C@@]1(F)O[C@@H](n2cc(Br)c3cncnc32)[C@H](O)[C@@H]1O] | ¹H NMR (400 MHz, D₂O): δ 8.91 (s, 1H), 8.81 (s, 1H), 7.82 (s, 1H), 6.65 (d, J = 2.8 Hz, 1H), 4.87-4.83 (m, 1H), 4.61-4.59 (m, 1H), 4.29-4.28 (m, 1H), 4.11-4.13 (m, 1H). ³¹P NMR (162 MHz, D₂O): δ −9.47-−9.43 (1P), −12.1-−12.0 (1P), −23.1-−22.8 (1P). ¹⁹F NMR (376 MHz, D₂O): δ −120.8 (1F). LCMS(ESI): 585.9 [M − H]⁻. |
| 80 | 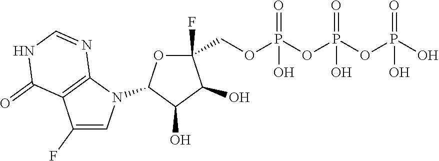 [SMILES: O=c1[nH]cnc2c1c(F)cn2[C@@H]1O[C@](F)(COP(=O)(O)OP(=O)(O)OP(=O)(O)O)[C@@H](O)[C@H]1O] | ¹H NMR (400 MHz, D₂O): δ 8.04 (s, 1H), 7.19 (s, 1H), 6.54 (s, 1H), 4.87-4.81 (m, 1H), 4.53-4.55 (m, 1H), 4.28-4.29 (m, 1H), 4.10-4.12 (m, 1H). ³¹P NMR (162 MHz, D₂O): δ −9.04- −8.92 (1P), −12.0-−11.9 (1P), −23.0-−22.7 (1P). ¹⁹F NMR (376 MHz, D₂O): δ −120.7 (1F), −163.8 (1F). LCMS(ESI): 542.0 [M − H]⁻. |
| 81 | 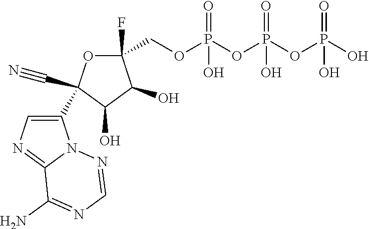 [SMILES: N#C[C@@]1(c2cnc3c(N)ncnn23)O[C@](F)(COP(=O)(O)OP(=O)(O)OP(=O)(O)O)[C@@H](O)[C@H]1O] | ¹H NMR (400 MHz, D₂O): δ 8.12 (s, 1H), 7.82 (s, 1H), , 4.86-4.88 (m, 1H), 4.58-4.63 (m, 1H), 4.17-4.25 (m, 2H). ³¹P NMR (162 MHz, D₂O): δ −10.1-−10.0 (1P), −12.2-−12.1 (1P), −23.2- −23.0 (1P). ¹⁹F NMR (376 MHz, D₂O): δ −120.9 (1F). LCMS(ESI): 549.0 [M − H]⁻. |
| 82 | 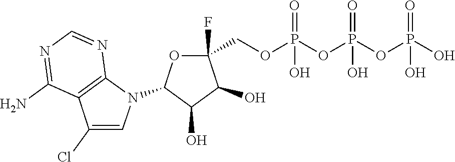 [SMILES: Nc1ncnc2c1c(Cl)cn2[C@@H]1O[C@](F)(COP(=O)(O)OP(=O)(O)OP(=O)(O)O)[C@@H](O)[C@H]1O] | ¹H NMR (400 MHz, D₂O): δ 7.96 (s, 1H), 7.29 (s, 1H),6.38 (d, J = 2.8 Hz, 1H), 4.65-4.43 (m, 1H), 4.43-4.41 (m, 1H), 4.18-4.16 (m, 1H), 4.00-3.97(m, 1H). ³¹P NMR (162 MHz, D₂O): δ −9.4-−9.36 (1P), −12.2-−12.0 (1P), −23.1-−22.9 (1P). ¹⁹F NMR (376 MHz, D₂O): δ −120.7 (1F). LCMS(ESI): 557.0 [M − H]⁻. |
| 83 | 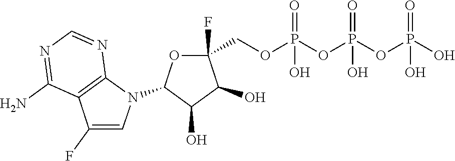 [SMILES: Nc1ncnc2c1c(F)cn2[C@@H]1O[C@](F)(COP(=O)(O)OP(=O)(O)OP(=O)(O)O)[C@@H](O)[C@H]1O] | ¹H NMR (400 MHz, D₂O): δ 8.01 (s, 1H), 7.12 (s, 1H), 6.46 (s, 1H), 4.71-7.69 (m, 1H), 4.45-4.46 (m, 1H), 4.08-4.23 (m, 2H). ³¹P NMR (162 MHz, D₂O): δ −10.3-−10.2 (1P), −12.2-−12.0 (1P), −23.2-−23.0 (1P). LCMS(ESI): 540.8 [M − H]⁻. |
| 84 | 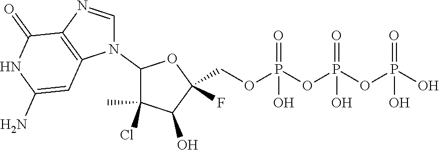 [SMILES: C[C@]1(Cl)C(n2cnc3c(=O)[nH]c(N)cc32)O[C@](F)(COP(=O)(O)OP(=O)(O)OP(=O)(O)O)[C@H]1O] | ¹H NMR (400 MHz, D₂O): δ 7.92 (s, 1H), 6.66 (s, 1H), 5.04-4.99 (m, 1H), 4.37-4.26 (m, 2H), 1.34 (s, 3H). ³¹P NMR (162 MHz, D₂O): δ −10.4-−10.6 (1P), −11.9-−12.0 (1P), −22.9-−23.1 (1P). ¹⁹F NMR (376 MHz, D₂O): δ −119.8 (1F). LCMS(ESI): 571.8 [M − H]⁻. |
| 85 | 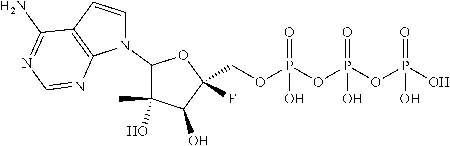 [SMILES: C[C@@]1(O)C(n2ccc3c(N)ncnc32)O[C@](F)(COP(=O)(O)OP(=O)(O)OP(=O)(O)O)[C@H]1O] | ¹H NMR (400 MHz, D₂O): δ 8.13 (s, 1H), 7.27 (d, J = 3.2 Hz, 1H), 6.61 (d, J = 3.2 Hz, 1H), 6.39 (s, 1H), 4.30-4.29 (m, 3H), 0.66 (s, 3H). ³¹P NMR (162 MHz, D₂O): 8 −9.23-−9.35 (1P), −12.04-−12.16 (1P), −22.63-−22.87 (1P). ¹⁹F NMR (376 MHz, D₂O): δ −120.5 (1F). LCMS(ESI): 537.0 [M − H]⁻. |

### Bioassays

### DENV-2 Viral Stock Generation

5E6 Vero cells (ATCC CCL-81) were plated in a T150 flask with complete DMEM (Gibco, 11965-092) containing 10% FBS, 1×NEAA (Thermofisher, 11140-050) 1× PenStrep (Thermofisher, 15140-122), overnight at 37° C. 5% CO₂. The media in the flask was removed and 10 mL of DENV-2 16681 (GenBank NC_001474.2) diluted in DMEM with 2% FBS was added to the flask at MOI of 0.01 and was allowed to incubate for one hour at 37° C. 5% CO₂. After incubation, 15 mL of complete DMEM was added and the flask was then incubated in a 33° C. incubator with 5% CO₂ for 15 days. The supernatant was harvested every two/three days, and 25 mL of new media was added to the flask. Specifically, the supernatant was harvested and centrifuged at 2,000×g for 5 minutes at 4° C., then aliquoted and stored at −80° C.

### Preparation of Zika Virus Stocks

5E6 Vero cells (ATCC CCL-81) were plated in a T150 flask with complete DMEM (Gibco, 11965-092) containing 10% FBS, 1×NEAA (Thermofisher, 11140-050) 1× PenStrep (Thermofisher, 15140-122), overnight at 37° C. 5% CO₂. The media in the flask was removed and 10 mL of ZIKV MR766 (BEI Resources NR-50065) diluted in DMEM with 2% FBS was added to the flask at MOI of 0.01 and was allowed to incubate for one hour at 37° C. 5% CO₂. After incubation, 15 mL of complete DMEM was added and the flask was incubated in a 37° C. incubator with 5% CO₂ for 3 days. The supernatant was then harvested and centrifuged at 2,000×g for 5 minutes at 4° C., then aliquoted and stored at −80° C.

### Preparation of Yellow Fever Virus Stocks

5E6 Vero cells (ATCC CCL-81) were plated in a T150 flask with complete DMEM (Gibco, 11965-092) containing 10% FBS, 1×NEAA (Thermofisher, 11140-050) 1× PenStrep (Thermofisher, 15140-122), overnight at 37° C. 5% CO₂. The media in the flask was removed and 10 mL of YFV strain 17D (BEI Resources NR-116) diluted in DMEM with 2% FBS was added to the flask at MOI of 0.01 and was allowed to incubate for one hour at 37° C. 5% CO₂. After incubation, 15 mL of complete DMEM was added and the flask was incubated in a 37° C. incubator with 5% CO₂ for 3 days. The supernatant was then harvested and centrifuged at 2,000×g for 5 minutes at 4° C., then aliquoted and stored at −80° C.

### Preparation of Chikungunva Virus Stocks

5E6 Vero cells (ATCC CCL-81) were plated in a T150 flask with complete DMEM (Gibco, 11965-092) containing 10% FBS, 1×NEAA (Thermofisher, 11140-050) 1× PenStrep (Thermofisher, 15140-122), overnight at 37° C. 5% CO₂. The media in the flask was removed and 10 mL of CHIKV strain 181/25 (BEI Resources NR-56523) diluted in DMEM with 2% FBS was added to the flask at MOI of 0.005 and was allowed to incubate for one hour at 37° C. 5% CO₂. After incubation, 20 mL of complete DMEM was added to the flask and the flask was incubated in a 37° C. incubator with 5% CO₂ for 3 days. The supernatant was then harvested and centrifuged at 2,000×g for 5 minutes at 4° C., then aliquoted and stored at −80° C.

### SARS-CoV-2 Viral Stock Generation

Vero E6 cells (ATCC CRL-1586) were plated in a T150 flask with complete DMEM (Corning 15-013-CV) containing 10% FBS, 1× PenStrep (Corning 20-002-CL), 2 mM L-Glutamine (Corning 25-005-CL) overnight at 37° C. 5% CO₂. The media in the flask was removed and 10 mL of SARS-CoV-2 strain USA-WA1/2020 (BEI Resources NR-52281) in complete DMEM was added to the flask at an MOI of 0.05 and was allowed to incubate for 30 minutes at 34° C. 5% CO₂. After incubation, 20 mL of complete DMEM was added to the flask. The flask was then placed in a 34° C. incubator at 5% CO₂. On day 3 post infection cells were scraped in the supernatant and the mixture was harvested and centrifuged at 2,000×g for 5 minutes. The cell pellet was freeze-thawed three times and then resuspended in the collected supernatant and centrifuged again at 2,000×g for 5 minutes at 4° C., then aliquoted and stored at −80° C.

### HeLa-ACE2 Stable Cell Line

HeLa-ACE2 cells were generated through transduction of human ACE2 lentivirus. The lentivirus was created by co-transfection of HEK293T cells with pBOB-hACE2 construct and lentiviral packaging plasmids pMDL, pREV, and pVSV-G (Addgene) using Lipofectamine 2000 (Thermo Fisher Scientific, 11668019). Supernatant was collected 48 h after transfection then used to transduce pre-seeded HeLa cells. 12 h after transduction stable cell lines were collected, scaled up and stored. Cells were maintained in DMEM (Gibco, 11965-092) with 10% FBS (Gibco, 10438026) and 1× sodium pyruvate (Gibco, 11360070) at 37° C. 5% CO₂.

### Preparation of RSV Virus.

To propagate RSV A2 (ATCC VR-1540), Hep-2 cells were infected at 80-90% confluency at an approximate MOI of 0.01 for 1-2 hours at 37° C. in a humidified 5% CO₂ atmosphere, rocking every 20-30 minutes to redistribute inoculum. Afterwards, the inoculum was removed and replaced with assay Medium (DMEM with 2% HI FBS, 1× Pen/Strep). The flasks were then incubated until significant CPE is observed typically 2-3 days post-infection. Supernatants were collected and clarified to remove cellular debris by centrifugation. The virus was then be stabilized by adding 25% sucrose and snap-freezing aliquots before storage at −80° C.

Preparation of seasonal coronavirus HCoV—OC43 and HCoV-229E Virus.

HCoV—OC43 was obtained through BEI Resources, NIAID, NIH: Human Coronavirus, OC43, NR-52725. To propagate the virus, HCT-8 cells were infected at an approximate MOI of 0.01 in RPMI for two hours at 33° C., 5% CO₂. After incubation, the inoculum was removed and replaced with Assay Medium (RPMI with 2% HI FBS, 1× Pen/Strep and 2 mM L-Glutamine). The flasks were incubated at 33° C., 5% CO₂ until significant CPE was observed, 3-4 days post-infection. The cells were scraped, harvested with the supernatant, and centrifuged at 1,000×g for 5 min. The supernatant was collected and the cell pellet subjected to one freeze-thaw cycle before being resuspended in 5 mL of supernatant and reclarified by centrifugation at 1,000×g for 5 min. The total supernatant was then pooled, aliquoted and stored at −80° C. HCoV-229E was obtained from BEI Resources, NR52726. To propagate the virus, MRC-5 pd25 were infected with HCoV-229E at an approximate MOI of 0.01 in MEM and allowed to incubate for two hours at 37° C. 5% CO₂. After incubation, the inoculum was removed and replaced with Assay Medium (MEM supplemented with 10% HI FBS, 2 mM L-glutamine, 1% NEAA, and 1× Pen/Strep). The flasks was then placed in a 37° C. incubator at 5% CO₂ until significant CPE was observed, 3-4 days post-infection and harvested above as described for OC43.

### Preparation of Human Rhinovirus Stocks.

Human Rhinovirus 16 strain 11757 (ATCC VR-283), 14 strain 1059 (ATCC VR-284), and 1B strain B632 (ATCC VR-1645) were propagated in H1 HeLa cells (ATCC CRL-1958). Cells were infected at an approximate MOI of 0.01 for 2 hrs in MEM at 33° C., 5% CO₂. The inoculum was removed and replaced with assay media (MEM+2% FBS+1XPS) and the flasks incubated for 2-3 days until significant CPE was observed. The cells were then be scraped, harvested with the supernatant, and centrifuged at 1,000×g for 5 min. The cell pellet was subjected to three freeze-thaw cycles before being resuspended in 5 mL of supernatant and reclarified by centrifugation. The total supernatant was then pooled, aliquoted and stored at −80° C.

### Preparation of Poliovirus Stocks.

Attenuated poliovirus strains PV-1 CHAT (VR-1562) and PV-3 WM-3 (ATCC VR-300) were propagated in HeLa S3 cells (ATCC CCL-2.2), follow a similar protocol as for HRV.

### DENV-2 HepG2 High-Content Screening Antiviral Assay

The antiviral activity of the compounds against DENV-2 was assessed in HepG2 (ATCC HB-8065) cells, using an image-based approach. Compounds were acoustically transferred into 384-well μclear-bottom plates (Greiner, Part. No. 781090-2B) using the Echo 555 Liquid Handler (LabCyte Inc). Cells were seeded in 20 μL DMEM with 2% FBS at a density of 2.0×10³ cells per well (HepG2). Plated cells were infected 4 hours later adding 10 μL of inoculum, DENV-2 diluted in assay media (MOI=0.5) to achieve ~50% infected cells. Plates were incubated for 24 h at 37° C. 5% CO₂, and then fixed with final concentration of 4% formaldehyde for 30 minutes at room temperature. Plates were washed twice with 1×PBS 0.05% Tween 20 in between fixation and subsequent primary and secondary antibody staining. Anti-Flavivirus Envelope (E) Protein [D1-4G2-4-15] Antibody (Millipore) diluted 1:2000 in PBST-0.3% BSA-0.2% saponin was added to the plate and incubated at 4° C. overnight. One μg/mL of Alexa Fluor 488 goat anti-mouse IgG H+L: (Thermo Fisher Scientific A-11001) together with 8 μM of antifade-46-diamidino-2-phenylindole (DAPI; Thermo Fisher Scientific D1306) diluted in PBST-0.3% BSA-0.2% saponin was added to each well and incubated at RT for 1 h in the dark. Plates were imaged using the ImageXpress Micro Confocal High-Content Imaging System (Molecular Devices) with a 10× objective, with 4 fields imaged per well. Images were analyzed using the Multi-Wavelength Cell Scoring Application Module (MetaXpress), with DAPI staining identifying the host-cell nuclei (the total number of cells in the images) and the DENV immunofluorescence signal leading to identification of infected cells.

### Zika Virus HepG2 High-Content Screening Antiviral Assay

The antiviral activity of compounds against ZIKV was assessed in HepG2 cells at 24 hrs post-infection using an image-based approach, following the essentially following the same protocol as described above for DENV-2, but using a dilution of ZIKV optimized to achieve ~50% infection 24 hrs as the inoculum.

### Yellow Fever Virus HepG2 High-Content Screening Antiviral Assay

The antiviral activity of compounds against YFV was assessed in HepG2 cells at 24 hrs post-infection using an image-based approach, following essentially the same protocol as described above for DENV-2 and ZIKV but using a dilution of Yellow Fever Virus as the inoculum.

### Chikungunya HepG2 High-Content Screening Antiviral Assay

The antiviral activity of compounds against Chikungunya was assessed in HepG2 at 24 hrs post-infection using an image-based approach and following a very similar protocol as described above for DENV-2 with the following substitutions: a dilution of Chikungunya virus was used as the inoculum and an anti-Chikungunya virus antibody was used as the primary antibody (CHIKV Antibody 11E7, monoclonal mouse IgG2b, Kerafast).

### SARS-CoV-2 HeLa-ACE2 High-Content Screening Assay

Compounds were acoustically transferred into 384-well μclear-bottom plates (Greiner, Part. No. 781090-2B) using the Echo 555 Liquid Handler (LabCyte Inc). HeLa-ACE2 cells were seeded in 13 μL DMEM with 2% FBS at a density of 1.0×10³ cells per well. Plated cells were transported to the BSL3 facility where 13 μL of SARS-CoV-2 diluted in assay media was added to achieve ~30-50% infected cells. Plates were incubated for 24 h at 34° C. 5% CO₂, and then fixed with final concentration of 4% formaldehyde for 1 h at 34° C. 5% CO₂. Plates were washed with 1×PBS 0.05% Tween 20 in between fixation and subsequent primary and secondary antibody staining. Human polyclonal plasma diluted 1:500 in Perm/Wash buffer (BD Biosciences 554723) was added to the plate and incubated at RT for 2 h. Six μg/mL of goat anti-human H+L conjugated Alexa 488 (Thermo Fisher Scientific A11013) together with 8 μM of antifade-46-diamidino-2-phenylindole (DAPI; Thermo Fisher Scientific D1306) in SuperBlock T20 (PBS) buffer (Thermo Fisher Scientific 37515) was added to the plate and incubated at RT for 1.5-2 hours in the dark. Plates were imaged using the ImageXpress Micro Confocal High-Content Imaging System (Molecular Devices) with a 10× objective, with 4 fields imaged per well. Images were analyzed using the Multi-Wavelength Cell Scoring Application Module (MetaXpress), with DAPI staining identifying the host-cell nuclei (the total number of cells in the images) and the SARS-CoV-2 immunofluorescence signal leading to identification of infected cells.

### OC-43 HCT-8 High-Content Screening Assay.

Compounds were acoustically transferred into 384-well μclear-bottom plates (Greiner, Part. No. 781089-2B) in dose-response using the Echo Liquid handler. HCT-8 cells were seeded into the assay-ready plates at 4000 cells in assay media (RPMI-1640+L-glutamine+2% FBS+1× Penicillin/Streptomycin solution). The plated cells were allowed to settle for 1 hour at 37° C., before being infected with HCoV—OC43 diluted in assay media to achieve ~30-60% infected cells. Plates were incubated for 48 h at 33° C. 5% CO₂ and then fixed with a final concentration of 4% formaldehyde. Fixed cells were blocked and permeabilized with Superblock and 0.2% Triton and then stained with mouse monoclonal antibody OC-43 strain clone 541-8F (Sigma Millipore MAB9012) overnight. This was followed by goat anti-mouse H+L conjugated Alexa 488 (Thermo Fisher Scientific A11001) and antifade-46-diamidino-2-phenylindole (DAPI; Thermo Fisher Scientific D1306), with PBS 0.05% Tween 20 washes in between fixation and subsequent primary and secondary antibody staining. Plates were then imaged and analyzed as for the SARS-CoV-2/HeLa-ACE2 assay.

### 229E/MRC5 CPE-Based Screening Assay.

Compounds were acoustically transferred into 384-well μclear-bottom plates (Greiner, Part. No. 781090-2B) in dose-response (1:3 serial dilutions starting at a final top assay concentration of 10 μM) using the Echo Liquid handler. MRC-5 PD25 cells were seeded into the assay ready plates at a density of 500 cells per well. The plated cells were allowed to settle for 1 hour at 37° C., before infection with hCoV-229E diluted in assay media. Plates were incubated for 120 h at 33° C. 5% CO₂. To assess the virus-induced cytopathic effect (CPE) through measurement of cell viability, 20 L of 50% Cell-Titer Glo (Promega No G7573) diluted in water was added to the cells and luminescence measured.

### HRV/H1 HeLa CPE-Based Screening Assay.

Compounds were acoustically transferred into either 384-well or 1536-well μclear-bottom plates (Greiner, Part. No. 781090-2B) in dose-response using the Echo Liquid handler. H1 HeLa cells were seeded into the assay ready plates at a density of 2000 cells per well in 384 or 600 cells per well in 1536. The plated cells were allowed to settle for 4 hours at 33° C., before infection with HRV 16, 14, or 1B diluted in assay media. Plates were incubated for 48 h at 33° C. 5% CO₂. To assess the virus-induced cytopathic effect (CPE) through measurement of cell viability, 50% Cell-Titer Glo (Promega No G7573) diluted in water was added to the cells and luminescence measured.

### PV HeLa S3 CPE-Based Screening Assay.

Compounds were acoustically transferred into 384-well μclear-bottom plates (Greiner, Part. No. 781090-2B) in dose-response using the Echo Liquid handler. HeLa S3 were seeded into the assay ready plates at a density of 4000 cells per well. The plated cells were allowed to settle for 4 hours at 37° C., before infection with either PV-1 CHAT or PV-3 WM-3 diluted in assay media. Plates were incubated for 48 hrs at 37° C. 5% CO₂. To assess the virus-induced cytopathic effect (CPE) through measurement of cell viability, 50% Cell-Titer Glo (Promega No G7573) diluted in water was added to the cells and luminescence measured.

### RSV Hep2 CPE-Based Screening Assay.

Compounds were acoustically transferred into 1536-well μclear-bottom plates (Greiner, Part. No. 781090-2B) in dose-response using the Echo Liquid handler. Hep2 were seeded into the assay ready plates at a density of 300 cells per well. The plated cells were allowed to settle for 1-2 hours at 37° C., before infection with RSV A2 diluted in assay media. Plates were incubated for 72 hrs. To assess the virus-induced cytopathic effect (CPE) through measurement of cell viability, 50% Cell-Titer Glo (Promega No G7573) diluted in water was added to the cells and luminescence measured.

### Uninfected Host Cell Cytotoxicity Counter Screens

The ability of compounds to cause cytotoxicity in uninfected cells was quantified through a cell viability assay based on ATP measurement. Briefly, for both the HepG2 and HeLa-ACE2 cells, compounds were acoustically transferred into 1,536-well black bottom plates (Greiner Part. No. 789176-F) using the Echo 555 Liquid Handler (LabCyte Inc). Cells were seeded in the assay-ready plates at 450 cells/well (HepG2) or 400 cells/well (Hela-ACE2) in 5 μL DMEM with 2% FBS and plates were incubated at 37° C. 5% CO₂ for either 72 h (HepG2) or 24 h (Hela-ACE2). To assess cell viability, 2 μL of 50% Cell-Titer Glo (Promega No G7573) diluted in water was added to the cells and luminescence measured using an PheraStar or ClarioStar Plate Reader (BMG LabTech).

### Data Analysis and Statistical Methods

The results for the DENV, ZIKV, YFV, CHIKV, SARS-CoV-2, HCoV—OC43, HCoV-229E, RSV, HRV, and PV infection assays and uninfected cytotoxicity counter screens data were uploaded to Genedata Screener, Version 16.0. For the High-content imaging assays, two outputs from the image analysis were analyzed: % W2 or virus Positive as the antiviral readout and Total cells. Data were normalized to neutral (DMSO) minus inhibitor controls (either 10 μM or 2.5 μM remdesivir for DENV, SARS-CoV2, HCoV—OC43, and RSV antiviral effect, and 10 μM puromycin dihydrochloride for infected host cell toxicity). For the uninfected host cell cytotoxicity counter screen, 30 μM puromycin dihydrochloride (Sigma) was used as the positive control. For the HCoV-229E, RSV, HRV, and PV cell viability/CPE readouts, data were normalized to Stimulator controls (2.5 μM Remdesivir for RSV and HCoV-229E, 1 μM AG-7404 for HRV) minus neutral controls (DMSO). For dose response experiments compounds were tested in technical triplicates and dose curves were fitted with the four parameter Hill Equation. EC₉₀ values were calculated in Excel from the EC50 and nHill values exported from GeneData Screener, according to the formula “POWER(9,1/nHill)*EC50”.

### Antiviral Profiling Through NIAID/Utah State

All other antiviral assays were run at Utah State/Justin Julander through NIAID Preclinical Services. The following virus strains and cell lines were used:

| Virus | Strain | Cell Line |
|---|---|---|
| MEV | CC | Huh7 |
| MERS-CoV | EMC | HEK |
| HRV14 | 1059 | HeLa OHIO |
| COXV-B3 | HA 201933 | HeLa OHIO |
| RSV | A2 | RD |
| CHIK V | S27 ATCC VR-64 | Huh7 |
| POV-1 | Mahoney | HeLa OHIO |
| DENV-2 | New Guinea C | Huh7 |
| Flu A H1N1 | California/07/2009 | MDCK |
| RVFV | MP12 | Huh7 |

Compounds were added to cells prior to addition of virus at an appropriate MOI to cause visible CPE. When maximal CPE was observed in virus controls, cells were stained with neutral red dye for a dye-uptake assay. These data were used to calculate EC50, CC50 and selective index (SI=CC50/EC50) for each compound. For compounds with an SI.5, a virus yield reduction assay was conducted, where supernatant collected from the CPE assay was titered for virus. The EC90 was calculated as the amount of compound needed to reduce the virus titer by 1 log 10.

The following data tables display biological assay data using the range codes shown below:
EC₅₀: <0.1 μM: ****
0.1-<1 μM: ***
1-<10 μM **
>10 μM *
CC₅₀: >99: ####
10-<99: ###
1-<1O: ##
<1: #

| Example | | | | |
|---|---|---|---|---|
| # / Table | DENV-2 / HepG2 | | SARS-CoV-2 / HeLa-ACE2 | |
| Order | EC₅₀ (μM) | CC₅₀ (uM) | EC₅₀ (μM) | CC₅₀ (uM) |
| 1 | **** | ## | **** | ## |
| 2 | * | #### | * | #### |
| 3 | * | #### | * | #### |
| 4 | **** | ### | ** | ### |
| 5 | *** | #### | * | #### |
| 6 | * | #### | * | #### |
| 7 | * | #### | * | #### |
| 8 | * | #### | * | #### |
| 9 | * | #### | * | #### |
| 10 | * | #### | * | #### |
| 11 | * | ### | * | #### |
| 12 | * | #### | * | #### |
| 13 | **** | ## | *** | ### |
| 14 | **** | # | *** | # |
| 15 | **** | # | **** | # |
| 16 | * | #### | * | #### |
| 17 | * | ### | * | #### |
| 18 | * | #### | * | #### |
| 19 | *** | # | ** | ## |
| 20 | * | #### | * | ### |
| 21 | * | #### | * | #### |
| 22 | *** | ## | ** | ## |
| 23 | *** | ## | ** | ## |
| 24 | **** | ## | *** | ## |
| 25 | **** | # | *** | # |
| 26 | **** | # | *** | # |
| 27 | * | ### | * | ### |
| 28 | *** | ## | * | ## |
| 29 | * | #### | * | #### |
| 30 | * | ### | * | ### |
| 31 | *** | # | ** | ## |
| 32 | * | #### | * | #### |
| 33 | **** | # | **** | ## |
| 34 | ** | # | * | ## |
| 35 | * | ### | * | #### |
| 36 | **** | # | *** | ## |
| 37 | * | #### | * | #### |
| 38 | ** | ## | * | # |
| 39 | * | #### | * | #### |
| 40 | * | #### | * | #### |
| 41 | * | ### | * | ### |
| 42 | **** | # | | |
| 43 | * | ### | * | #### |
| 44 | * | #### | * | #### |
| 45 | * | #### | * | #### |
| 46 | **** | ## | *** | ## |
| 47 | **** | ## | ** | ### |
| 48 | *** | ### | ** | ### |
| 49 | ** | #### | * | #### |
| 50 | *** | ## | * | ### |
| 51 | * | #### | * | #### |
| 52 | ** | # | ** | ## |
| 53 | ** | ### | | |
| 54 | * | #### | | |
| 55 | | | **** | ## |
| 56 | * | #### | * | #### |
| 58 | *** | #### | * | #### |
| 59 | ** | #### | * | #### |
| 60 | * | #### | * | #### |
| 61 | * | #### | | |
| 62 | *** | ### | * | #### |
| 63 | * | ### | * | #### |
| 64 | ** | ### | * | ### |
| 65 | ** | ## | ** | ### |
| 66 | * | #### | * | #### |
| 67 | ** | ### | * | ### |
| 68 | * | ### | * | #### |
| 69 | ** | ### | * | ### |
| 70 | * | ### | * | #### |
| 71 | * | #### | * | #### |
| 72 | ** | ### | ** | ### |

| Example | | | | | |
|---|---|---|---|---|---|
| # / Table | RSV A2 / Hep2 | | HRV / H1 HeLa | | |
| Order | EC₅₀ (μM) | CC₅₀ (uM) | B 14 EC₅₀ (μM) | A16 EC₅₀ (μM) | CC₅₀ (uM) |
| 1 | **** | ## | *** | *** | ## |
| 3 | * | #### | * | | #### |
| 4 | *** | ### | *** | *** | ### |
| 5 | * | #### | ** | | #### |
| 6 | * | #### | * | | #### |
| 7 | * | #### | * | | #### |
| 8 | * | #### | * | | #### |
| 9 | * | #### | * | | #### |
| 10 | * | #### | * | * | #### |
| 11 | * | #### | * | | ### |
| 12 | * | #### | * | | ### |
| 13 | *** | ### | ** | *** | ### |
| 14 | **** | ## | * | * | ## |
| 15 | * | ## | * | **** | # |
| 16 | * | #### | * | * | #### |
| 18 | * | #### | * | * | #### |
| 19 | * | ## | * | * | ## |
| 20 | * | ### | * | * | #### |
| 21 | * | #### | * | * | #### |
| 22 | * | ### | * | * | ### |
| 23 | ** | ### | ** | ** | ### |
| 24 | *** | ### | ** | * | ### |
| 25 | *** | ### | * | * | ## |
| 26 | **** | # | * | * | ## |
| 27 | * | ### | * | * | ### |
| 28 | *** | ## | * | | ## |
| 29 | * | | * | * | #### |
| 30 | *** | ## | * | * | ### |
| 31 | * | # | * | * | ## |
| 32 | * | #### | * | * | #### |
| 33 | **** | ### | * | * | ## |
| 34 | *** | # | * | * | ## |
| 35 | * | ### | * | | ### |
| 36 | ** | ### | * | | ### |
| 37 | * | #### | * | | #### |
| 38 | * | ### | * | | ### |
| 39 | * | #### | * | | #### |
| 41 | * | ### | * | * | ### |
| 43 | * | #### | * | * | ### |
| 44 | * | ### | * | * | ### |
| 45 | * | #### | * | * | #### |
| 46 | *** | ### | *** | | ### |
| 47 | **** | ## | ** | | ### |
| 48 | | | ** | ** | ### |
| 49 | | | ** | * | #### |
| 50 | * | ### | * | * | ### |
| 51 | * | #### | * | | #### |
| 52 | * | ## | ** | | ## |
| 53 | * | #### | * | | #### |
| 54 | * | #### | * | | #### |
| 56 | * | #### | * | * | #### |
| 58 | * | #### | *** | | #### |
| 59 | * | #### | ** | | #### |
| 60 | * | #### | * | | #### |
| 62 | * | ### | * | | ### |
| 63 | * | #### | * | | #### |
| 64 | * | ### | * | | ### |
| 65 | * | ### | * | | ### |
| 66 | * | #### | * | | #### |
| 67 | * | ### | ** | | ### |
| 68 | * | ### | * | | ### |
| 69 | * | ### | * | | ### |
| 70 | * | ### | * | | ### |
| 71 | * | #### | * | | #### |
| 72 | * | ### | * | | #### |

| Example | | | |
|---|---|---|---|
| # / Table | ZIKV / HepG2 | YFV / HepG2 | CHIKV / HepG2 |
| Order | EC₅₀ (μM) | EC₅₀ (μM) | EC₅₀ (μM) |
| 1 | **** | **** | **** |
| 2 | | | * |
| 4 | *** | **** | *** |
| 5 | ** | ** | |
| 13 | | | *** |
| 17 | | | * |
| 19 | | | *** |
| 20 | | | * |
| 21 | * | * | |
| 22 | ** | *** | |
| 24 | *** | *** | |
| 25 | **** | **** | |
| 30 | | | * |
| 31 | | | *** |
| 32 | | | * |
| 35 | * | ** | |
| 40 | * | * | |
| 48 | ** | ** | |

| Example | HCoV-OC43/HCT-8 | | HCoV-229E/MRC5 | | HCMV/HFF | |
|---|---|---|---|---|---|---|
| #/Table | EC₅₀ | CC₅₀ | EC₅₀ | CC₅₀ | EC₅₀ | CC₅₀ |
| Order | (μM) | (uM) | (μM) | (uM) | (μM) | (uM) |
| 1 | *** | ## | *** | ### | ** | ## |
| 4 | *** | ### | * | #### | * | ### |
| 6 | | | | | * | #### |
| 7 | | | | | * | #### |
| 13 | ** | ### | * | #### | * | ### |
| 14 | *** | ## | * | ## | *** | ## |
| 15 | **** | # | *** | ## | ** | ## |
| 16 | | | | | * | #### |
| 20 | | | | | * | #### |
| 21 | * | #### | * | #### | | |
| 22 | | | | | ** | #### |
| 24 | *** | ### | * | #### | * | ### |
| 25 | *** | # | * | #### | ** | #### |
| 33 | *** | # | * | ### | *** | ### |
| 36 | | | | | *** | ## |
| 60 | | | | | * | #### |
| 63 | | | | | * | |
| 66 | | | | | * | |
| 69 | | | | | * | |
| 71 | | | | | * | |

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A compound of Formula (I) 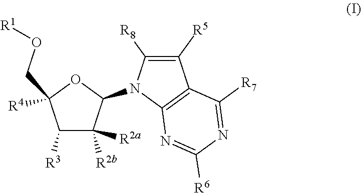
wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR^{1′})NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR^{1′})NH(C₁-C₆)alkyl(C₅-C₈)heteroaryl, —P(═O)(NHR^{1′})NH(C₁-C₆)alkyl, —P(═O)(NHR¹′)NH(C₁-C₆)heteroalkyl, —P(═O)(NHR^{1′})NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR^{1′})NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R^{2a} is H, halo, (C₁-C₆)alkyl, or —C≡CH;
R^{2b} is independently halo or OH;
R³ is independently H or OH;
R⁴ is N₃, halo, —C≡N, (C₁-C₃)haloalkyl or —O(C₁-C₆)alkyl;
R⁵ is H, halo, —C≡N, (C₁-C₆)alkyl, hetero (C₁-C₆)alkyl, hydroxy (C₁-C₆)alkyl, N(R¹′)₂, —C(═O)NH₂;
R⁶ is H, halo, NH₂, (C₁-C₆)alkyl, (C₂-C₆)alkenyl, or (C₂-C₆)alkynyl;
R⁷ is H, NH₂, OH, oxo, halo, N(R¹′)₂ or —O(C₁-C₆)alkyl; and
R⁸ is H or halo;
with the proviso that the compound of Formula I is not 4-amino-7-((2R,3R,4S,5S)-5-fluoro-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5R)-5-azido-3,4-dihydroxy-5-(hydroxymethyl)tetrahydrofuran-2-yl)-2-methyl-7H-pyrrolo[2,3-d]pyrimidine-5-carboxamide; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carbonitrile; ((5-(4-amino-7H-pyrrolo[2,3-d]pyrimidin-7-yl)-4-ethynyl-2-fluoro-3,4-dihydroxytetrahydrofuran-2-yl)methyl)triphosphoric acid; ((5-(4-amino-5-fluoro-7H-pyrrolo[2,3-d]pyrimidin-7-yl)-4-ethynyl-2-fluoro-3,4-dihydroxytetrahydrofuran-2-yl)methyl)triphosphoric acid; (5-(4-amino-5-carbamoyl-7H-pyrrolo[2,3-d]pyrimidin-7-yl)-4-ethynyl-2-fluoro-3,4-dihydroxytetrahydrofuran-2-yl)methyl tetrahydrogen triphosphate; (5-(4-amino-5-cyano-7H-pyrrolo[2,3-d]pyrimidin-7-yl)-4-ethynyl-2-fluoro-3,4-dihydroxytetrahydrofuran-2-yl)methyl tetrahydrogen triphosphate; 4-amino-7-((2R,3R,4S,5S)-3-ethynyl-5-fluoro-3,4-dihydroxy-5-(hydroxymethyl)tetrahydrofuran-2-yl)-7H-pyrrolo[2,3-d]pyrimidine-5-carboxamide; (2S,3S,4R,5R)-5-(4-amino-7H-pyrrolo[2,3-d]pyrimidin-7-yl)-4-ethynyl-2-fluoro-2-(hydroxymethyl)tetrahydrofuran-3,4-diol; or (2S,3 S,4R,5R)-5-(4-amino-5-fluoro-7H-pyrrolo[2,3-d]pyrimidin-7-yl)-4-ethynyl-2-fluoro-2-(hydroxymethyl)tetrahydrofuran-3,4-diol;
including enantiomers, racemic and scalemic mixtures, and further including pharmaceutically acceptable salts thereof.

2. A compound of Formula (II) 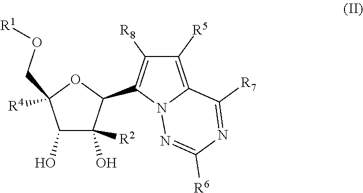
wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR¹′)NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R² is H, (C₁-C₆)alkyl, or —C≡CH;
R⁴ is N₃, halo, or —O(C₁-C₆)alkyl;
R⁵ is H, halo, —C≡N, hetero (C₁-C₆)alkyl, hydroxy (C₁-C₆)alkyl, N(R¹′)₂, —C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, N(R¹′)₂ or —O(C₁-C₆)alkyl; and
R⁸ is H or halo;
with the proviso that the compound of Formula II is not (2S,3S,4R,5S)-5-(4-aminopyrrolo[2,1-f][1,2,4]triazin-7-yl)-2-fluoro-2-(hydroxymethyl)tetrahydrofuran-3,4-diol, (2R,3 S,4R,5S)-5-(4-aminopyrrolo[2,1-f][1,2,4]triazin-7-yl)-2-azido-2-(hydroxymethyl)tetrahydrofuran-3,4-diol, (2R,3S,4R,5S)-5-(4-amino-5-fluoropyrrolo[2,1-f][1,2,4]triazin-7-yl)-2-azido-2-(hydroxymethyl)tetrahydrofuran-3,4-diol; (2R,3S,4R,5S)-5-(4-aminopyrrolo[2,1-f][1,2,4]triazin-7-yl-5-d)-2-azido-2-(hydroxymethyl)tetrahydrofuran-3,4-diol; ((2R,3S,4R,5S)-5-(4-aminopyrrolo[2,1-f][1,2,4]triazin-7-yl)-2-azido-3,4-dihydroxytetrahydrofuran-2-yl)methyl tetrahydrogen triphosphate, or 2-ethylbutyl ((((2R,3S,4R,5S)-5-(4-aminopyrrolo[2,1-f][1,2,4]triazin-7-yl)-2-azido-3,4-dihydroxytetrahydrofuran-2-yl)methoxy)(phenoxy)phosphoryl)-D-alaninate;
including enantiomers, racemic and scalemic mixtures, and further including pharmaceutically acceptable salts thereof.

3. A compound of Formula (IIIa) or (IIIb) 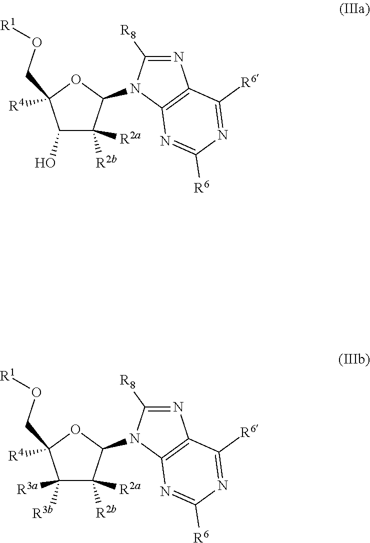
wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R^{2a} is H, OH, halo, (C₁-C₆)alkyl, or —C≡CH;
R^{2b} is H, OH, halo, or (C₁-C₆)alkyl;
R^{3a} is H, OH, halo, or (C₁-C₆)alkyl;
R^{3b} is H, OH, halo, or (C₁-C₆)alkyl;
R⁴ is N₃, halo, —C≡N, (C₁-C₃)haloalkyl or —O(C₁-C₆)alkyl;
R⁶ and R^{6′} are each independently halo, NH₂, NH(C₁-C₆)alkyl, N((C₁-C₆)alkyl)₂, —OH, —O(C₁-C₆)alkyl, -oxo, or —C≡CH; and
R⁸ is H or halo;
with the proviso that the compound of Formula III is not (((2R,3S,4R,5R)-5-(2,6-diamino-9H-purin-9-yl)-2-(difluoromethyl)-3,4-dihydroxytetrahydrofuran-2-yl)methyl)triphosphoric acid; (((2S,3S,4R,5R)-5-(6-amino-2-fluoro-9H-purin-9-yl)-4-ethynyl-2-fluoro-3,4-dihydroxytetrahydrofuran-2-yl)methyl)triphosphoric acid; (2S,3S,4R,5R)-5-(2,6-diamino-9H-purin-9-yl)-2-fluoro-2-(hydroxymethyl)-4-methyltetrahydrofuran-3,4-diol; or (2S,3S,4R,5R)-5-(6-amino-2-fluoro-9H-purin-9-yl)-4-ethynyl-2-fluoro-2-(hydroxymethyl)tetrahydrofuran-3,4-diol;
including enantiomers, racemic and scalemic mixtures, and further including pharmaceutically acceptable salts thereof.

4. A compound of Formula (IV) 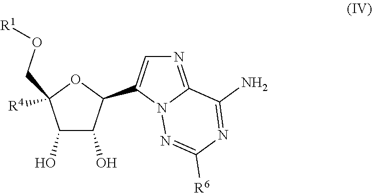
wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR^{1′})NH(C₁-C₆)alkyl, —P(═O)(OR¹′)NH(C₁-C₆)heteroalkyl, —P(═O)(OR^{1′})₂, —P(═O)(OR^{1′})—P(═O)(OR^{1′})₂, —P(═O)(OR¹′)—P(═O)(OR¹′)—P(═O)(OR¹′)₂, P(═O)(OR^{1′})NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR^{1′})NH(C₆-C₁₀)aryl, —P(═O)(OR^{1′})NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR^{1′})NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R⁴ is N₃, halo, or —O(C₁-C₆)alkyl; and
R⁶ is H, halo, (C₁-C₆)alkyl, (C₂-C₆)alkenyl, or (C₂-C₆)alkynyl;
including enantiomers, racemic and scalemic mixtures, and further including pharmaceutically acceptable salts thereof.

5. A compound of Formulae (V) 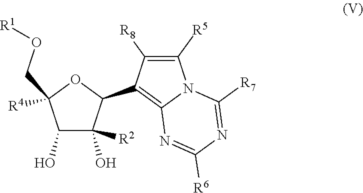
wherein:
R¹ is selected from the group consisting of H, —C(═O)(C₁-C₆)alkyl, —C(═O)(C₁-C₆)heteroalkyl, —C(═O)(C₃-C₇)cycloalkyl, —C(═O)(C₁-C₆)alkyl(C₃-C₇)cycloalkyl, —C(═O)(C₃-C₇)heterocycloalkyl, —C(═O)(C₁-C₆)alkyl(C₃-C₇)heterocycloalkyl, —C(═O)(C₆-C₁₀)aryl, —C(═O)(C₁-C₆)alkyl(C₆-C₁₀)aryl, —C(═O)(C₅-C₈)heteroaryl, —C(═O)(C₁-C₆)alkyl(C₅-C₈)heteroaryl, —C(═O)CH(NH₂)(C₁-C₆)alkyl, —C(═O)CH(NH(C₁-C₆)alkyl)(C₁-C₆)alkyl, —C(═O)CH(N((C₁-C₆)alkyl)₂)(C₁-C₆)alkyl, —C(═O)CH(NH₂)(C₁-C₆)alkyl(C₆-C₁₀)aryl, —C(═O)CH(NH(C₁-C₆)alkyl)(C₁-C₆)alkyl(C₆-C₁₀)aryl, —C(═O)CH(N((C₁-C₆)alkyl)₂)(C₁-C₆)alkyl(C₆-C₁₀)aryl, —P(═O)(OR¹′)₂, —P(═O)(OR¹′)—P(═O)(OR¹′)₂, —P(═O)(OR¹′)—P(═O)(OR¹′)—P(═O)(OR¹′)₂, —P(═O)(OR¹′)NH(C₁-C₆)alkyl, —P(═O)(OR¹′)NH(C₁-C₆)heteroalkyl, —P(═O)(OR¹′)NH(C₁-C₆)haloalkyl, —P(═O)(OR¹′)NH(C₃-C₇)cycloalkyl, —P(═O)(OR¹′)NH(C₃-C₇)heterocycloalkyl, —P(═O)(OR¹′)NH(C₆-C₁₀)aryl, —P(═O)(OR¹′)NH(C₁-C₆)alkyl(C₆-C₁₀)aryl, —P(═O)(OR¹′)NH(C₅-C₈)heteroaryl, —P(═O)(OR¹′)NH(C₁-C₆)alkyl(C₅-C₈)heteroaryl, —P(═O)(NHR¹′)NH(C₁-C₆)alkyl, —P(═O)(NHR¹′)NH(C₁-C₆)heteroalkyl, —P(═O)(NHR¹′)NH(C₁-C₆)haloalkyl, —P(═O)(NHR¹′)NH(C₃-C₇)cycloalkyl, —P(═O)(NHR¹′)NH(C₃-C₇)heterocycloalkyl, —P(═O)(NHR¹′)NH(C₆-C₁₀)aryl, —P(═O)(NHR¹′)NH(C₁-C₆)alkyl(C₆-C₁₀)aryl, —P(═O)(NHR¹′)NH(C₅-C₈)heteroaryl, —P(═O)(NHR¹′)NH(C₁-C₆)alkyl(C₅-C₈)heteroaryl, —P(═O)(OR¹′)NH(C₁-C₆)alkyl-C(═O)O—(C₁-C₆)alkyl, —P(═O)(OR¹′)NH(C₁-C₆)heteroalkyl-C(═O)O—(C₁-C₆)alkyl, —P(═O)(OR¹′)NH(C₁-C₆)haloalkyl-C(═O)O—(C₁-C₆)alkyl, —P(═O)(OR¹′)NH(C₁-C₆)alkyl-C(═O)O—(C₁-C₆)alkyl(C₆-C₁₀)aryl, —P(═O)(OR¹′)NH(C₁-C₆)heteroalkyl-C(═O)O—(C₁-C₆)alkyl(C₆-C₁₀)aryl, —P(═O)(OR¹′)NH(C₁-C₆)haloalkyl-C(═O)O—(C₁-C₆)alkyl(C₆-C₁₀)aryl, —P(═O)(OR¹′)NH(C₁-C₆)alkyl-C(═O)O—(C₆-C₁₀)aryl, —P(═O)(OR¹′)NH(C₁-C₆)heteroalkyl-C(═O)O—(C₆-C₁₀)aryl, and —P(═O)(OR¹′)NH(C₁-C₆)haloalkyl-C(═O)O—(C₆-C₁₀)aryl;
each R¹′ is independently H, —(C₁-C₆)alkyl, —(C₁-C₆)heteroalkyl, —(C₁-C₆)haloalkyl, —(C₂-C₆)alkenyl, —(C₂-C₆)heteroalkenyl, —(C₂-C₆)haloalkenyl, —(C₂-C₆)alkynyl, —(C₂-C₆)heteroalkynyl, —(C₂-C₆)haloalkynyl, —(C₃-C₇)cycloalkyl, —(C₃-C₇)heterocycloalkyl, —(C₆-C₁₀)aryl, —(C₁-C₆)alkyl(C₆-C₁₀)aryl, —(C₅-C₈)heteroaryl, or —(C₁-C₆)alkyl(C₅-C₈)heteroaryl;
R² is H, halo, (C₁-C₆)alkyl, or —C≡CH;
R⁴ is N₃, halo, —C≡N, (C₁-C₃)haloalkyl or —O(C₁-C₆)alkyl;
R⁵ is H, halo, —C≡N, hetero (C₁-C₆)alkyl, hydroxy (C₁-C₆)alkyl, halo (C₁-C₆)alkyl, N(R¹′)₂, —(C(═O)NH₂;
R⁶ is H, halo, NH₂, (C₁-C₆)alkyl, (C₂-C₆)alkenyl, or (C₂-C₆)alkynyl;
R⁷ is H, NH₂, OH, halo, N(R¹′)₂, or —O(C₁-C₆)alkyl; and
R⁸ is H or halo;
including enantiomers, racemic and scalemic mixtures, and further including pharmaceutically acceptable salts thereof.

6. The compound of claim 1, wherein R^{2b} is OH or halo.

7. The compound of claim 3, wherein R^{2b} is halo.

8. The compound of claim 7, wherein R^{2a} is Me or halo.

9. The compound of any one of claims 6-8, wherein R^{2a} is H.

10. The compound of any one of claims 6-8, wherein R^{2a} is (C₁-C₆)alkyl.

11. The compound of claim 10, wherein R^{2a} is Me.

12. The compound of any one of claims 6-8, wherein R^{2a} is halo.

13. The compound of claim 12, wherein R^{2a} is Cl.

14. The compound of any one of claims 2 or 5 wherein R² is H.

15. The compound of any one of claims 2 or 5, wherein R² is halo.

16. The compound of claim 15, wherein R² is F.

17. The compound of any one of claims 2 or 5, wherein R² is (C₁-C₆)alkyl.

18. The compound of claim 17, wherein R² is Me.

19. The compound of any one of claims 1-2 or 5, wherein R⁵ is H.

20. The compound of any one of claims 1-2 or 5, wherein R⁵ is —CH₂OH.

21. The compound of any one of claims 1-2 or 5, wherein R⁵ is —C(═O)NH₂.

22. The compound of any one of claims 1, 6-13, or 19-21, wherein R³ is OH.

23. The compound of any one of claims 1, 6-13, or 19-21, wherein R³ is H.

24. The compound of any one of claims 1-23, wherein R⁴ is halo.

25. The compound of claim 24, wherein R⁴ is F.

26. The compound of claim 24, wherein R⁴ is Cl.

27. The compound of any one of claims 1-23 wherein R⁴ is N₃.

28. The compound of any one of claims 1-23, wherein R⁴ is —O(C₁-C₆)alkyl.

29. The compound of claim 28, wherein R⁴ is —OMe.

30. The compound of any one of claims 1-29, wherein R⁶ is H or NH₂ and R^{6′} is —NH(C₁-C₆)alkyl or —O(C₁-C₆)alkyl.

31. The compound of any one of claims 1-29, wherein R⁶ is halo.

32. The compound of claim 31, wherein R⁶ is F.

33. The compound of any one of claims 1-29, wherein R⁶ is —C≡CH.

34. The compound of any one of claims 1-33, wherein R¹ is H.

35. The compound of any one of claims 1-33, wherein R¹ is —P(═O)(OR¹′)₂, —P(═O)(OR¹′)—P(═O)(OR¹′)₂, or —P(═O)(OR¹′)—P(═O)(OR¹′)—P(═O)(OR¹′)₂.

36. The compound of any one of claims 1-33, wherein R¹ is —P(═O)(OR″)NH(C₁-C₆)alkyl-C(═O)O—(C₁-C₆)alkyl.

37. A compound having any one of the formulae selected from the group consisting of:
(2S,3S,4R,5R)-5-(4-amino-7H-pyrrolo[2,3-d]pyrimidin-7-yl)-2-fluoro-2-(hydroxymethyl)tetrahydrofuran-3,4-diol;
(2R,3S,4R,5R)-5-(4-amino-7H-pyrrolo[2,3-d]pyrimidin-7-yl)-2-azido-2-(hydroxymethyl)tetrahydrofuran-3,4-diol;
(2R,3S,4R,5S)-5-(4-aminopyrrolo[2,1-f][1,2,4]triazin-7-yl)-2-(hydroxymethyl)-2-methoxy-4-methyltetrahydrofuran-3,4-diol;
(2S,3S,4R,5R)-5-(4-chloro-7H-pyrrolo[2,3-d]pyrimidin-7-yl)-2-fluoro-2-(hydroxymethyl)tetrahydrofuran-3,4-diol;
(2R,3S,4R,5R)-5-(4-chloro-7H-pyrrolo[2,3-d]pyrimidin-7-yl)-2-fluoro-2-(hydroxymethyl)tetrahydrofuran-3,4-diol;
(2S,3R,4S,5R)-5-(4-amino-7H-pyrrolo[2,3-d]pyrimidin-7-yl)-4-chloro-2,4-difluoro-2-(hydroxymethyl)tetrahydrofuran-3-ol;
(2R,3R,4S,5R)-5-(4-amino-7H-pyrrolo[2,3-d]pyrimidin-7-yl)-2-azido-4-chloro-4-fluoro-2-(hydroxymethyl)tetrahydrofuran-3-ol;
(2R,3R,5R)-2-(4-amino-7H-pyrrolo[2,3-d]pyrimidin-7-yl)-5-azido-5-(hydroxymethyl)tetrahydrofuran-3-ol;
(2R,3S,4R,5S)-5-(4-aminopyrrolo[2,1-f][1,2,4]triazin-7-yl)-2-azido-2-(hydroxymethyl)-4-methyltetrahydrofuran-3,4-diol;
(2S,3S,4R,5R)-5-(4-amino-7H-pyrrolo[2,3-d]pyrimidin-7-yl)-2-fluoro-2-(hydroxymethyl)-4-methyltetrahydrofuran-3,4-diol;
(2R,3R,5R)-2-(4-amino-7H-pyrrolo[2,3-d]pyrimidin-7-yl)-5-fluoro-5-(hydroxymethyl)tetrahydrofuran-3-ol;
(2R,3R,5S)-2-(4-amino-7H-pyrrolo[2,3-d]pyrimidin-7-yl)-5-fluoro-5-(hydroxymethyl)tetrahydrofuran-3-ol;
(2S,3S,4R,5R)-2-fluoro-5-(4-fluoro-7H-pyrrolo[2,3-d]pyrimidin-7-yl)-2-(hydroxymethyl)tetrahydrofuran-3,4-diol;
4-amino-7-((2R,3R,4S,5S)-5-fluoro-3,4-dihydroxy-5-(hydroxymethyl)tetrahydrofuran-2-yl)-7H-pyrrolo[2,3-d]pyrimidine-5-carboxamide;
(2S,3S,4R,5R)-5-(4-amino-5-fluoro-7H-pyrrolo[2,3-d]pyrimidin-7-yl)-2-fluoro-2-(hydroxymethyl)tetrahydrofuran-3,4-diol;
7-((2R,3R,4S,5S)-5-fluoro-3,4-dihydroxy-5-(hydroxymethyl)tetrahydrofuran-2-yl)-3,7-dihydro-4H-pyrrolo[2,3-d]pyrimidin-4-one;
(2S,3S,4R,5R)-5-(4-amino-5-(hydroxymethyl)-7H-pyrrolo[2,3-d]pyrimidin-7-yl)-2-fluoro-2-(hydroxymethyl)tetrahydrofuran-3,4-diol;
(2S,3S,4R,5R)-5-(6-amino-2-ethynyl-9H-purin-9-yl)-2-fluoro-2-(hydroxymethyl)tetrahydrofuran-3,4-diol;
(2S,3S,4R,5S)-5-(4-aminoimidazo[2,1-f][1,2,4]triazin-7-yl)-2-fluoro-2-(hydroxymethyl)tetrahydrofuran-3,4-diol;
(2R,3 S,4R,5R)-5-(4-amino-7H-pyrrolo[2,3-d]pyrimidin-7-yl)-2-(fluoromethyl)-2-(hydroxymethyl)tetrahydrofuran-3,4-diol;
5-fluoro-7-((2R,3R,4S,5S)-5-fluoro-3,4-dihydroxy-5-(hydroxymethyl)tetrahydrofuran-2-yl)-3,7-dihydro-4H-pyrrolo[2,3-d]pyrimidin-4-one;
(2S,3S,4R,5R)-2-fluoro-5-(5-fluoro-4-methoxy-7H-pyrrolo[2,3-d]pyrimidin-7-yl)-2-(hydroxymethyl)tetrahydrofuran-3,4-diol;
(2S,3S,4R,5R)-2-fluoro-2-(hydroxymethyl)-5-(7H-pyrrolo[2,3-d]pyrimidin-7-yl)tetrahydrofuran-3,4-diol;
7-((2R,3R,4S,5S)-5-fluoro-3,4-dihydroxy-5-(hydroxymethyl)tetrahydrofuran-2-yl)-7H-pyrrolo[2,3-d]pyrimidine-5-carboxamide;
(2S,3S,4R,5R)-5-(5-bromo-7H-pyrrolo[2,3-d]pyrimidin-7-yl)-2-fluoro-2-(hydroxymethyl)tetrahydrofuran-3,4-diol;
4-amino-7-((2R,3R,4S,5S)-5-fluoro-3,4-dihydroxy-5-(hydroxymethyl)tetrahydrofuran-2-yl)-7H-pyrrolo[2,3-d]pyrimidine-5-carbonitrile;
(2S,3S,4R,5R)-5-(2-amino-4-chloro-7H-pyrrolo[2,3-d]pyrimidin-7-yl)-2-fluoro-2-(hydroxymethyl)tetrahydrofuran-3,4-diol;
(2S,3S,4R,5R)-2-fluoro-2-(hydroxymethyl)-5-(6-(methylthio)-9H-purin-9-yl)tetrahydrofuran-3,4-diol;
(2S,3S,4R,5R)-5-(4-chloro-2-methyl-7H-pyrrolo[2,3-d]pyrimidin-7-yl)-2-fluoro-2-(hydroxymethyl)tetrahydrofuran-3,4-diol;
(1S,2S,3S,5R)-5-(4-amino-7H-pyrrolo[2,3-d]pyrimidin-7-yl)-3-fluoro-3-(hydroxymethyl)cyclopentane-1,2-diol;
(2S,3S,4R,5R)-5-(6-amino-2-fluoro-9H-purin-9-yl)-2-fluoro-2-(hydroxymethyl)tetrahydrofuran-3,4-diol;
(3S,4R,5R)-5-(4-amino-7H-pyrrolo[2,3-d]pyrimidin-7-yl)-3,4-dihydroxy-2-(hydroxymethyl)tetrahydrofuran-2-carbonitrile;
(2S,3S,4R,5R)-5-(4-amino-5-chloro-7H-pyrrolo[2,3-d]pyrimidin-7-yl)-2-fluoro-2-(hydroxymethyl)tetrahydrofuran-3,4-diol;
((2S,3S,4R,5S)-5-(4-amino-5-carbamoylpyrrolo[2,1-f][1,2,4]triazin-7-yl)-2-fluoro-3,4-dihydroxytetrahydrofuran-2-yl)methyl benzoate;
(2S,3S,4R,5R)-2-fluoro-5-(5-fluoro-4-(methylamino)-7H-pyrrolo[2,3-d]pyrimidin-7-yl)-2-(hydroxymethyl)tetrahydrofuran-3,4-diol;
(2S,3S,4R,5R)-5-(5-chloro-7H-pyrrolo[2,3-d]pyrimidin-7-yl)-2-fluoro-2-(hydroxymethyl)tetrahydrofuran-3,4-diol;
7-((2R,3R,4S,5S)-5-fluoro-3,4-dihydroxy-5-(hydroxymethyl)tetrahydrofuran-2-yl)-4-oxo-4,7-dihydro-3H-pyrrolo[2,3-d]pyrimidine-5-carbonitrile;
7-((2R,3R,4S,5S)-5-fluoro-3,4-dihydroxy-5-(hydroxymethyl)tetrahydrofuran-2-yl)-4-methoxy-7H-pyrrolo[2,3-d]pyrimidine-5-carboxamide;
(2R,3R,5R)-5-fluoro-5-(hydroxymethyl)-2-(4-methoxy-7H-pyrrolo[2,3-d]pyrimidin-7-yl)tetrahydrofuran-3-ol;
(2S,3S,4R,5R)-2-fluoro-2-(hydroxymethyl)-5-(4-methoxy-7H-pyrrolo[2,3-d]pyrimidin-7-yl)tetrahydrofuran-3,4-diol;
(2S,3S,4R,5R)-5-(4-amino-5-(trifluoromethyl)-7H-pyrrolo[2,3-d]pyrimidin-7-yl)-2-fluoro-2-(hydroxymethyl)tetrahydrofuran-3,4-diol;
(2S,3S,4R,5R)-5-(6-amino-2-chloro-9H-purin-9-yl)-2-fluoro-2-(hydroxymethyl)tetrahydrofuran-3,4-diol;
4-amino-1-((2R,3R,4S,5S)-3-ethynyl-5-fluoro-3,4-dihydroxy-5-(hydroxymethyl)tetrahydrofuran-2-yl)pyrimidin-2(1H)-one;
(2S,3S,4R,5R)-2-((benzoyloxy)methyl)-5-(3,5-dioxo-4,5-dihydro-1,2,4-triazin-2(3H)-yl)-2-fluorotetrahydrofuran-3,4-diyl diacetate;
((2S,3S,4R,5R)-5-(4-amino-7H-pyrrolo[2,3-d]pyrimidin-7-yl)-2-fluoro-3,4-dihydroxytetrahydrofuran-2-yl)methyl isobutyrate;
(2S,3S,4R,5R)-5-(4-amino-7H-pyrrolo[2,3-d]pyrimidin-7-yl)-2-fluoro-2-((isobutyryloxy)methyl)tetrahydrofuran-3,4-diyl bis(2-methylpropanoate);
isopropyl ((S)-(((2S,3S,4R,5R)-5-(4-amino-7H-pyrrolo[2,3-d]pyrimidin-7-yl)-2-fluoro-3,4-dihydroxytetrahydrofuran-2-yl)methoxy)(phenoxy)phosphoryl)-L-alaninate;
isopropyl ((R)-(((2S,3S,4R,5R)-5-(4-amino-7H-pyrrolo[2,3-d]pyrimidin-7-yl)-2-fluoro-3,4-dihydroxytetrahydrofuran-2-yl)methoxy)(phenoxy)phosphoryl)-L-alaninate;
((2S,3S,4R,5R)-4-(benzoyloxy)-5-(4-cyano-7H-pyrrolo[2,3-d]pyrimidin-7-yl)-2-fluoro-3-hydroxytetrahydrofuran-2-yl)methyl benzoate;
(2R,3R,4S,5S)-2-(4-aminoimidazo[2,1-f][1,2,4]triazin-7-yl)-5-fluoro-3,4-dihydroxy-5-(hydroxymethyl)tetrahydrofuran-2-carbonitrile;
(2S,3S,4R,5R)-5-(4-amino-1H-pyrazolo[3,4-d]pyrimidin-1-yl)-2-fluoro-2-(hydroxymethyl)tetrahydrofuran-3,4-diol;
(2S,3S,4R,5R)-2-fluoro-2-(hydroxymethyl)-5-(4-methyl-7H-pyrrolo[2,3-d]pyrimidin-7-yl)tetrahydrofuran-3,4-diol;
(2S,3S,4R,5R)-5-(4-cyclopropyl-7H-pyrrolo[2,3-d]pyrimidin-7-yl)-2-fluoro-2-(hydroxymethyl)tetrahydrofuran-3,4-diol;
(2S,3R,5R)-5-(4-amino-5-(benzo[d]thiazol-2-yl)-7H-pyrrolo[2,3-d]pyrimidin-7-yl)-2,4,4-trifluoro-2-(hydroxymethyl)tetrahydrofuran-3-ol;
((2S,3S,4R,5R)-5-(2-amino-4-(methylamino)-7H-pyrrolo[2,3-d]pyrimidin-7-yl)-2-fluoro-3,4-dihydroxytetrahydrofuran-2-yl)methyl benzoate;
(2S,3R,4S,5R)-5-(2-amino-6-(methylamino)-9H-purin-9-yl)-4-chloro-2,4-difluoro-2-(hydroxymethyl)tetrahydrofuran-3-ol;
isopropyl ((R)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate;
isopropyl ((S)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate;
(2S,3R,5R)-5-(2-amino-6-ethoxy-9H-purin-9-yl)-2,4,4-trifluoro-2-(hydroxymethyl)tetrahydrofuran-3-ol;
isopropyl ((((2S,3R,5R)-5-(2-amino-6-oxo-1,6-dihydro-9H-purin-9-yl)-2,4,4-trifluoro-3-hydroxytetrahydrofuran-2-yl)methoxy)(phenoxy)phosphoryl)-L-alaninate;
2-amino-9-((2R,3R,4R,5 S)-3-chloro-5-fluoro-4-hydroxy-5-(hydroxymethyl)-3-methyltetrahydrofuran-2-yl)-1,9-dihydro-6H-purin-6-one;
(2S,3R,4R,5R)-5-(2-amino-6-ethoxy-9H-purin-9-yl)-4-chloro-2-fluoro-2-(hydroxymethyl)-4-methyltetrahydrofuran-3-ol;
isopropyl ((((2S,3R,4R,5R)-5-(2-amino-6-ethoxy-9H-purin-9-yl)-4-chloro-2-fluoro-3-hydroxy-4-methyltetrahydrofuran-2-yl)methoxy)(phenoxy)phosphoryl)-L-alaninate;
neopentyl ((((2S,3S,4R,5R)-5-(4-aminoimidazo[2,1-f][1,2,4]triazin-7-yl)-5-cyano-2-fluoro-3,4-dihydroxytetrahydrofuran-2-yl)methoxy)(naphthalen-1-yloxy)phosphoryl)-L-alaninate;
(2S,3R,4S,5R)-5-(2-amino-6-ethoxy-9H-purin-9-yl)-4-chloro-2,4-difluoro-2-(hydroxymethyl)tetrahydrofuran-3-ol;
isopropyl ((((2S,3R,4S,5R)-5-(2-amino-6-ethoxy-9H-purin-9-yl)-4-chloro-2,4-difluoro-3-hydroxytetrahydrofuran-2-yl)methoxy)(phenoxy)phosphoryl)-L-alaninate;
(2S,3R,4S,5R)-5-(2-amino-6-ethoxy-9H-purin-9-yl)-4-bromo-2,4-difluoro-2-(hydroxymethyl)tetrahydrofuran-3-ol;
isopropyl ((((2S,3R,5R)-5-(2-amino-6-ethoxy-9H-purin-9-yl)-2,4,4-trifluoro-3-hydroxytetrahydrofuran-2-yl)methoxy)(phenoxy)phosphoryl)-L-alaninate;
(2S,3S,4R,5R)-4-ethynyl-2-fluoro-2-(hydroxymethyl)-5-(4-(propylamino)-7H-pyrrolo[2,3-d]pyrimidin-7-yl)tetrahydrofuran-3,4-diol;
2-amino-9-((2R,4R,5S)-3,3,5-trifluoro-4-hydroxy-5-(hydroxymethyl)tetrahydrofuran-2-yl)-1,9-dihydro-6H-purin-6-one;
(2S,3S,4R,5R)-2-fluoro-2-(hydroxymethyl)-5-(4-(methylthio)-7H-pyrrolo[2,3-d]pyrimidin-7-yl)tetrahydrofuran-3,4-diol;
((2S,3S,4R,5R)-5-(4-amino-7H-pyrrolo[2,3-d]pyrimidin-7-yl)-2-fluoro-3,4-dihydroxytetrahydrofuran-2-yl)methyl L-valinate;
neopentyl ((((2S,3S,4R,5R)-5-(4-amino-1H-pyrazolo[3,4-d]pyrimidin-1-yl)-2-fluoro-3,4-dihydroxytetrahydrofuran-2-yl)methoxy)(naphthalen-1-yloxy)phosphoryl)-L-alaninate;
((2S,3S,4R,5R)-5-(4-amino-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(4-chloro-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-2-fluoro-3,4-dihydroxy-5-(4-oxo-3,4-dihydro-7H-pyrrolo[2,3-d]pyrimidin-7-yl)tetrahydrofuran-2-yl)methyl tetrahydrogen triphosphate;
((2S,3S,4R,5R)-5-(5-carbamoyl-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(5-bromo-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-2-fluoro-5-(5-fluoro-4-oxo-3,4-dihydro-7H-pyrrolo[2,3-d]pyrimidin-7-yl)-3,4-dihydroxytetrahydrofuran-2-yl)methyl tetrahydrogen triphosphate;
((2S,3S,4R,5R)-5-(4-aminoimidazo[2,1-f][1,2,4]triazin-7-yl)-5-cyano-2-fluoro-3,4-dihydroxytetrahydrofuran-2-yl)methyl tetrahydrogen triphosphate;
((2S,3S,4R,5R)-5-(4-amino-5-chloro-7H-pyrrolo[2,3-d]pyrimidin-7-yl)-2-fluoro-3,4-dihydroxytetrahydrofuran-2-yl)methyl tetrahydrogen triphosphate;
((2S,3S,4R,5R)-5-(4-amino-5-fluoro-7H-pyrrolo[2,3-d]pyrimidin-7-yl)-2-fluoro-3,4-dihydroxytetrahydrofuran-2-yl)methyl tetrahydrogen triphosphate;
((2S,3R,4R,5R)-5-(2-amino-6-oxo-1,6-dihydro-9H-purin-9-yl)-4-chloro-2-fluoro-3-hydroxy-4-methyltetrahydrofuran-2-yl)methyl tetrahydrogen triphosphate; and
((2S,3S,4S,5R)-5-(4-amino-7H-pyrrolo[2,3-d]pyrimidin-7-yl)-2-fluoro-3,4-dihydroxy-4-methyltetrahydrofuran-2-yl)methyl tetrahydrogen triphosphate.

38. The compound of claim 37 having the formula (2S,3S,4R,5R)-5-(4-amino-7H-pyrrolo[2,3-d]pyrimidin-7-yl)-2-fluoro-2-(hydroxymethyl)tetrahydrofuran-3,4-diol or isopropyl ((R)-(((2S,3R,4S,5R)-5-(2-amino-6-(methylamino)-9H-purin-9-yl)-4-chloro-2,4-difluoro-3-hydroxytetrahydrofuran-2-yl)methoxy)(phenoxy)phosphoryl)-L-alaninate.

39. A pharmaceutical composition comprising the compound of any one of claims 1-38, admixed with a pharmaceutically acceptable carrier, diluent, or excipient.

40. The pharmaceutical composition of claim 39, further comprising one or more therapeutic compounds or compositions.

41. The pharmaceutical composition of claim 40, wherein the one or more therapeutic compounds or compositions is a second antiviral compound or composition.

42. The pharmaceutical composition of claim 41, wherein the second antiviral compound or composition is an RdRp inhibitor.

43. The pharmaceutical composition of claim 41, wherein the second antiviral compound or composition is and RNA polymerase inhibitor.

44. A method of inhibiting RNA-dependent RNA polymerases, comprising administering to a subject in need thereof a therapeutically effective amount of the compound of any one of claims 1-38 or the pharmaceutical compositions of claims 39-43.

45. A method of preventing, ameliorating, or treating an RNA viral infection, comprising administering to a subject in need thereof a therapeutically effective amount of the compound of any one of claims 1-38 or the pharmaceutical compositions of claims 39-43.

46. The method of claim 45, wherein the RNA viral infection is at least one virus selected from the group consisting of Dengue virus, South Asian Respiratory syndrome-Coronavirus (SARS-CoV), SARS-COV-2, Zika virus, Yellow Fever virus, Ebola (Makona) virus, Ebola (Kikwit) virus, Bundibugyo virus, Sudan virus, Marburg virus, respiratory syncytial virus (RSV), Nipah virus, measles virus, parainfluenza virus, Middle Eastern Respiratory Syndrome (MERS) virus, hepatitis C virus (HCV), West Nile virus, Lassa virus, influenza, HRV, MEV, LCMV, polio, CHIKV, COXV and Junin virus.

47. The method of claim 46, wherein the RNA viral infection is caused by Dengue Fever virus.

48. The method of claim 46, wherein the RNA viral infection is caused by SARS-COV-2 virus.

49. The method of claim 46, wherein the RNA viral infection is caused by Yellow Fever virus.

50. The method of claim 46, wherein the RNA viral infection is caused by Zika virus.

51. The method of any one of claims 44-50, further comprising treatment with one or more additional therapeutic compounds or compositions.

52. The method of claim 51, wherein at least one of the one or more therapeutic compounds or compositions is a drug effective for treating or ameliorating RNA viral infections.

53. The method of claim 52, wherein the drug for treating an RNA viral infection is selected from the group consisting of remdesivir, molnupiravir, or paxlovid.

54. Any compound, composition, or method as described herein.
